(19) United States
(12) Patent Application Publication
(10) Pub. No.: US 2011/0166144 A1
(43) Pub. Date: Jul. 7, 2011

(54) Pyrimidotriazinediones and Pyrimidopyrimidinediones and Methods of Using the Same

(75) Inventors: H.D. H. Showalter, Ann Harbor, MI (US); Anjanette J. Turbiak, Canton, MI (US); Eric R. Fearon, Ann Arbor, MI (US); Guido T. Bommer, Ann Arbor, MI (US)

(73) Assignee: THE REGENTS OF THE UNIVERSITY OF MICHIGAN, Ann Arbor (US)

(21) Appl. No.: 13/055,220

(22) PCT Filed: Jul. 30, 2009
(86) PCT No.: PCT/US09/52235

§ 371 Date: Mar. 22, 2011

Related U.S. Application Data
(62) Division of application No. 61/085,070, filed on Jul. 31, 2008.

(51) Int. Cl. A61K 31/53; C07D 487/04; A61P 35/00

(52) U.S. Cl. 514/243; 544/184

(57) ABSTRACT

The present disclosure is directed to pyrimidotriazinediones and pyrimidopyrimidinediones having a formula (I), (II), or (III), or a mixture or pharmaceutically acceptable salt or hydrate thereof, and to methods of treating cancer comprising administering the same.

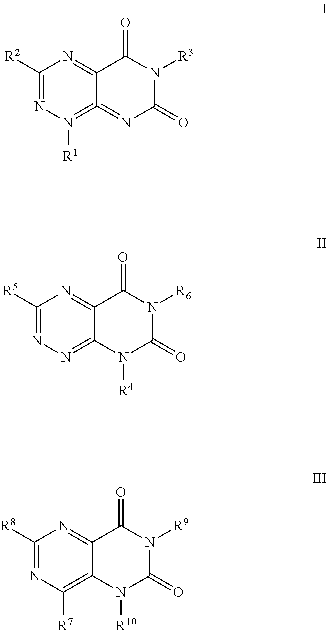

### BACKGROUND

1. Field of the Disclosure

The disclosure relates generally to bicyclic heterocyclic compounds. More particularly, the disclosure relates to substituted pyrimidotriazinediones and substituted pyrimidopyrimidinediones and to methods of using such compounds in the treatment of diseases and disorders.

2. Brief Description of Related Technology

The Wnt signaling pathway plays critical roles in processes as diverse as embryonic development, stem cell growth, and tumorigenesis. Various Wnt family members are known, and although their biological functions vary, the majority of Wnt family members can activate a signaling pathway with β-catenin as the central effector. β-catenin is a multifunctional protein that binds to and activates the Tcf/LEF family of transcription factors. A multiprotein complex referred to as the β-catenin destruction complex regulates β-catenin levels in the cytosol. In the absence of a Wnt signal, β-catenin is phosphorylated, ubiquitinated, and degraded by the proteasome. In the presence of a Wnt signal, however, the destruction complex is inhibited. Inhibition of the destruction complex allows the level of β-catenin to rise, resulting in the translocation of β-catenin into the nucleus, where it then interacts with members of the Tcf/LEF family of transcription factors to induce transcription of target genes.

Increased β-catenin levels frequently are associated with colorectal tumors and a variety of other cancers. Antagonists capable of reducing β-catenin-mediated transcriptional activation are theorized to have anti-neoplastic effects that may be useful in anti-cancer therapies. Furthermore, due to the involvement of Wnt signaling in the maintenance of the stem cell compartments of numerous organs, it is theorized that temporary administration of weakly toxic inhibitors can offer protection of stem cell compartments during treatment with high dose conventional chemotherapy. Some inhibitors of β-catenin-mediated transcriptional activation, for example, can reduce binding of β-catenin to Tcf/LEF transcription factors such as Tcf4, which is highly expressed in colorectal cancers, thereby reducing β-catenin-mediated transcription. Lepourcelet et al. (Cancer Cell, 5:91-102 (2004)), for example, reported the use of a high-throughput screen to identify several small molecule antagonists of the oncogenic Tcf4/β-catenin protein complex.

### SUMMARY

Disclosed herein are bicyclic heterocycles that are useful for treating cell proliferative disorders such as cancer, for example, colorectal cancers.

The present disclosure relates to methods of treating cancer comprising administering a compound having a formula I, II, or III, or a mixture or pharmaceutically acceptable salt or hydrate thereof:

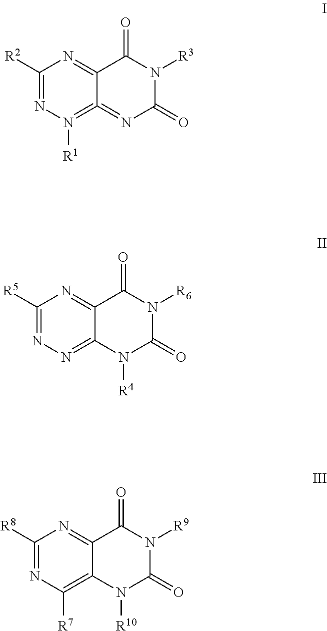

wherein R¹, R³, R⁴, R⁶, R⁷, R⁸, R⁹, and R¹⁰ are each independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl;
R² is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, and optionally substituted heteroaryl; and
R⁵ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, optionally substituted heteroaryl, optionally substituted C₁₋₂₀ alkylamino, optionally substituted C₁₋₂₀ alkoxy, and optionally substituted C₁₋₂₀ alkylcarboxamido;
with the proviso that R² is different from hydrogen when both R¹ and R³ are methyl.

The disclosure also relates to compounds having a formula II, or a pharmaceutically acceptable salt or hydrate thereof:

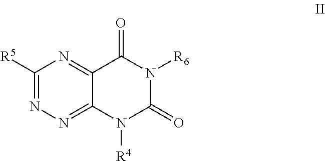

wherein R⁴ and R⁶ are independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl; and
R⁵ is selected from the group consisting of optionally substituted C₉₋₂₀ alkyl, optionally substituted C₇₋₂₀ alkenyl, optionally substituted C₂₋₂₀ alkynyl, optionally substituted C₇₋₂₀ cycloalkyl, optionally substituted C₇₋₂₀ heterocycloalkyl, optionally substituted amino(C₁₋₂₀ alkoxy)phenyl, optionally substituted amino(C₁₋₂₀ alkylamino)phenyl, optionally substituted amino(C₁₋₂₀ alkyl)carboxamidophenyl, optionally substituted amino(C₁₋₂₀ alkoxy)heteroaryl, optionally substituted amino(C₁₋₂₀ alkylamino)heteroaryl, optionally substituted amino(C₁₋₂₀ alkyl)carboxamido heteroaryl, optionally substituted amino(C₁₋₂₀ alkylamino), optionally substituted amino(C₁₋₂₀ alkoxy), optionally substituted amino(C₁₋₂₀ alkyl)carboxamido, optionally substituted amino(C₁₋₂₀ alkyl)amino(C₁₋₂₀ alkyl), and optionally substituted amino(C₁₋₂₀ alkyl)acylamino(C₁₋₂₀ alkyl).

In another embodiment, the disclosure relates to compounds having a formula II, or a pharmaceutically acceptable salt or hydrate thereof:

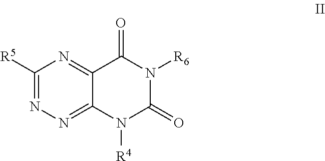

wherein R⁴ is selected from the group consisting of C₇₋₂₀ alkyl, substituted C₁₋₂₀ alkyl, C₉₋₂₀ alkenyl, substituted C₂₋₂₀ alkenyl, optionally substituted C₂₋₂₀ alkynyl, C₇₋₂₀ cycloalkyl, substituted C₃₋₂₀ cycloalkyl, optionally substituted C₂₋₂₀ heterocycloalkyl, substituted aryl, and optionally substituted heteroaryl;
with the proviso that R⁴ is different from benzyl;
R⁵ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, optionally substituted heteroaryl, optionally substituted C₁₋₂₀ alkylamino, optionally substituted C₁₋₂₀ alkoxy, and optionally substituted C₁₋₂₀ alkylcarboxamido; and
R⁶ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl.

The disclosure also relates to compounds having a formula or III, or a pharmaceutically acceptable salt or hydrate thereof:

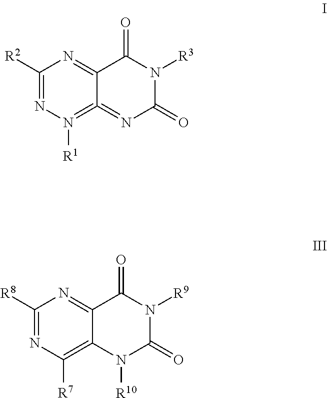

wherein R¹, R², and R³ are each independently selected From the group consisting of optionally substituted aryl and optionally substituted heteroaryl;
R⁷, R⁸, R⁹, and R¹⁰ are each independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl;
with the proviso that when R⁷ is hydrogen and R⁸ is phenyl, para-methoxyphenyl, fury/, or —CO₂Et, at least one of R⁹ and R¹⁰ is different from methyl.

### DETAILED DESCRIPTION

The present disclosure is directed to bicyclic heterocycles that are useful for the treatment of cancers, such as colorectal cancer. The compounds include substituted pyrimido[5,4-e][1,2,4]triazine-5,7(1H,6H)-diones, substituted pyrimido[5,4-e][1,2,4]triazine-5,7(6H,8H)-diones, and substituted pyrimido[5,4-d]pyrimidine-2,4(1H,3H)-diones. Methods are provided for treating diseases and disorders, such as a cancer (e.g., colorectal cancers, melanoma, anaplastic thyroid carcinoma, ovarian cancer), comprising administering a therapeutically effective amount of a compound disclosed herein to a mammal in need thereof. The disclosure also relates to methods of reducing β-catenin-mediated transcriptional activation, and to methods of reducing binding of β-catenin to Tcf/LEF transcription factors using the compounds disclosed herein.

As used herein, the term “alkyl” refers to straight chained and branched saturated hydrocarbon groups, nonlimiting examples of which include methyl, ethyl, and straight chain and branched propyl and butyl groups. Alkyl groups can have, for example, from 1 to 20 carbon atoms, from 1 to 10 carbon atoms, and/or from 1 to 6 carbon atoms. The term “alkyl” includes “bridged alkyl,” i.e., a bicyclic or polycyclic hydrocarbon groups, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (—OH), oxo (═O), halo, aryl, heteroaryl, cycloalkyl, heterocycloalkyl, amino, nitro (NO₂), and thio.

As used herein, the term “alkenyl” refers to straight chained and branched hydrocarbon groups containing at least one carbon-carbon double bond, nonlimiting examples of which include ethenyl, and straight chain and branched propenyl and butenyl groups. Alkenyl groups can have, for example, from 2 to 20 carbon atoms, from 2 to 10 carbon atoms, and/or from 2 to 6 carbon atoms. Alkenyl groups optionally can be substituted, for example, with one or more substituents previously listed as optional alkyl substituents.

As used herein, the term “alkynyl” refers to straight chained and branched hydrocarbon groups containing at least one carbon-carbon triple bond, nonlimiting examples of which include ethynyl, and straight chain and branched propynyl and butynyl groups. Alkynyl groups can have, for example, from 2 to 20 carbon atoms, from 2 to 10 carbon atoms, and/or from 2 to 6 carbon atoms. Alkynyl groups optionally can be substituted, for example, with one or more substituents previously listed as optional alkyl substituents.

As used herein, the term “cycloalkyl” refers to a cyclic C₃₋₂₀ hydrocarbon group, e.g., cyclopropyl, cyclobutyl, cyclohexyl, and cyclopentyl. “Heterocycloalkyl” is defined similarly as cycloalkyl, except the ring contains one or more heteroatoms, for example, one to three heteroatoms, independently selected from the group consisting of oxygen, nitrogen, and sulfur. Nonlimiting examples of heterocycloalkyl groups include piperidinyl, tetrahydrofuranyl, tetrahydropyranyl, dihydrofuranyl, and the like. Cycloalkyl and heterocycloalkyl groups can be saturated or partially unsaturated ring systems optionally substituted, for example, with alkyl, alkylene —OH, —C(O)NH₂, —NH₂, —NO₂, oxo (═O), aryl, haloalkyl, halo, —OH and —SH. Heterocycloalkyl groups optionally can be further N-substituted with alkyl, hydroxyalkyl, alkylenearyl, or alkyleneheteroaryl.

As used herein, the term “alkylene” refers to an alkyl group having a substituent. For example, the term “alkylene heterocycloalkyl” refers to an alkyl group substituted with a heterocycloalkyl group. Alkylene groups can have, for example, from 1 to 20 carbon atoms, from 1 to 10 carbon atoms, and/or from 1 to 6 carbon atoms. The alkylene group optionally can be substituted, for example, with one or more substituents previously listed as optional alkyl substituents.

As used herein, the term “alkenylene” refers to an alkenyl group having a substituent. For example, the term “alkenylene heterocycloalkyl” refers to an alkenyl group substituted with a heterocycloalkyl group. Alkenylene groups can have, for example, from 2 to 20 carbon atoms, from 2 to 10 carbon atoms, and/or from 2 to 6 carbon atoms. The alkenylene group optionally can be substituted, for example, with one or more substituents previously listed as optional alkyl substituents.

As used herein, the term “alkynylene” is defined identically as “alkylene,” except the group contains at least one carbon-carbon triple bond, refers to an alkynyl group having a substituent. For example, the term “alkynylene heterocycloalkyl” refers to an alkynyl group substituted with a heterocycloalkyl group. Alkynylene groups can have, for example, from 2 to 20 carbon atoms, from 2 to 10 carbon atoms, and/or from 2 to 6 carbon atoms. The alkynylene group optionally can be substituted, for example, with one or more substituents previously listed as optional alkyl substituents.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, —OCF₃, —NO₂, —CN, —NC, —OH, alkoxy, amino, —CO₂H, —CO₃-alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or polycyclic aromatic group, preferably a monocyclic or bicyclic aromatic group, containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, —CO₂H, —CO₂-alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

As used herein, the term “halo” refers to the halogens of Group VIIA of the periodic table, such as F, Cl, Br, and I.

As used herein, the term “amino” refers to an —NH) group wherein one or both of the hydrogen atoms optionally can be substituted, for example, with alkyl, substituted alkyl, cycloalkyl, aryl, or heteroaryl.

As used herein, the term “alkoxy” refers to an —OR group wherein R is optionally substituted alkyl, optionally substituted alkenyl, optionally substituted alkynyl, optionally substituted cycloalkyl, optionally substituted aryl, or optionally substituted heteroaryl.

As used herein, the term “thio” refers to an —SH group wherein the hydrogen atom optionally can be substituted, for example, with alkyl, substituted alkyl, cycloalkyl, aryl, or heteroaryl.

As used herein, the term “acylamino” refers to an —NHC(═O)R group wherein the hydrogen atom optionally can be substituted, for example, with alkyl, substituted alkyl, cycloalkyl, aryl, or heteroaryl, and R is optionally substituted alkyl, optionally substituted alkenyl, optionally substituted alkynyl, optionally substituted cycloalkyl, optionally substituted aryl, or optionally substituted heteroaryl.

As used herein, the term “alkylcarboxamido” refers to a —C(═O)NRR′ group wherein R is hydrogen, optionally substituted alkyl, optionally substituted alkenyl, optionally substituted alkynyl, optionally substituted cycloalkyl, optionally substituted aryl, or optionally substituted heteroaryl, and R′ is optionally substituted alkyl, optionally substituted alkenyl, optionally substituted alkynyl, or optionally substituted cycloalkyl, or R and R′ taken together with the nitrogen atom to which they are bound form an optionally substituted heterocyclic ring.

Additionally, salts of the compounds disclosed herein also are included in the present disclosure and can be used in the methods disclosed herein. For example, an acid salt of a compound containing an amine or other basic group can be obtained, by reacting the compound with a suitable organic or inorganic acid, such as hydrogen chloride, hydrogen bromide, acetic acid, perchloric acid and the like. Examples of such salts include, but are not limited to, hydrochlorides, hydrobromides, sulfates, methanesulfonates, nitrates, maleates, acetates, citrates, fumarates, tartrates, succinates, benzoates and salts with amino acids such as glutamic acid. Salts of compounds containing a carboxylic acid or other acidic functional group can be prepared by reacting with a suitable base. Such salts include, but are not limited to, alkali metal salts (e.g., sodium and potassium), alkaline earth metal salts (e.g., calcium and magnesium), aluminum salts ammonium salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethypamine, procaine, dibenzylpiperidine, dehydroabietylamine, bisdehydroabietylamine, glucamine, N-methylglucamine, collidine, quinine, quinoline, and basic amino acids such as lysine and arginine.

The present disclosure relates to methods of treating cancer comprising administering to a mammal in need thereof a therapeutically effective amount of a compound having a formula I, II, or III, or a mixture or pharmaceutically acceptable salt or hydrate thereof:

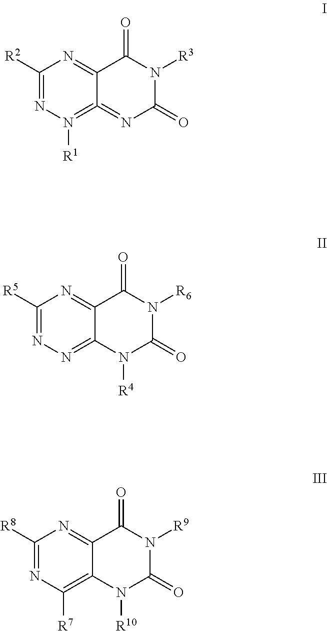

wherein R¹, R³, R⁴, R⁶, R⁷, R⁸, R⁹, and R¹⁰ are each independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl;
R² is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, and optionally substituted heteroaryl; and
R⁵ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, optionally substituted heteroaryl, optionally substituted C₁₋₂₀ alkylamino, optionally substituted C₁₋₂₀ alkoxy, and optionally substituted C₁₋₂₀ alkylcarboxamido;
with the proviso that R² is different from hydrogen when both R¹ and R³ are methyl.

The compounds disclosed herein are named according to the following position assignments:

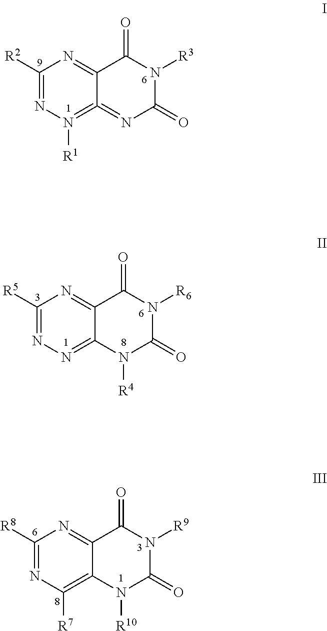

Those skilled in the art appreciate that the compounds defined by formulae I, II, and III can exist in tautomeric forms. All of the tautomeric forms of the compounds also are included in the scope of the present disclosure.

Compounds of formula I include, but are not limited to, compounds wherein R¹ is selected from the group consisting of hydrogen, C₁₋₂₀ alkyl, C₁₋₂₀ alkylene-OH, C₁₋₂₀ alkylene-NR^{a}R^{b}, and optionally substituted C₁₋₂₀ alkylene-aryl, wherein R^{a} and R^{b} are each independently selected from the group consisting of hydrogen and C₁₋₂₀ alkyl. Exemplary compounds of formula I include compounds wherein R¹ is hydrogen, methyl, 2-hydroxyethyl, benzyl, fluorobenzyl, difluorobenzyl, phenyl, propyl, cyclopentyl, and cyclopropyl.

Compounds of formula I also include, but are not limited to, compounds wherein R² is selected from the group consisting of hydrogen, C₁₋₂₀ alkyl, optionally substituted heteroaryl, and optionally substituted C₁₋₂₀ alkylene-aryl. Exemplary compounds of formula I include compounds wherein R² is hydrogen, methyl, ethyl, pyridinyl, 6-(2-(diethylamino)ethoxy)pyridyl-3-yl, and methylfuryl.

Compounds of formula I further include, but are not limited to, compounds wherein R³ is selected from the group consisting of hydrogen, C₁₋₂₀ alkyl, C₁₋₂₀ alkylene-OH, C₁₋₂₀ alkylene-NR^{a}R^{b}, and optionally substituted C₁₋₂₀ alkylene-aryl, wherein R^{a} and R^{b} are each independently selected from the group consisting of hydrogen and C₁₋₂₀ alkyl or R^{a} and R^{b} taken together with the nitrogen atom form a 3 to 10 membered heterocyclic ring. Exemplary compounds of formula I include compounds wherein R³ is hydrogen, methyl, 2-hydroxyethyl, 2-(piperidin-1-yl)ethyl, and C₁₋₃ alkylene-aryl, such as fluorobenzyl, difluorobenzyl, and (2-aminoethyl)benzyl.

Compounds of formula II include, but are not limited to, compounds wherein R⁴ is selected from the group consisting of hydrogen, C₁₋₂₀ alkyl, C₁₋₂₀ alkylene-OH, C₁₋₂₀ alkylene-NR^{a}R^{b}, optionally substituted C₁₋₂₀ alkylene-aryl, and optionally substituted C₁₋₂₀ alkylene-heteroaryl, wherein R^{a} and R^{b} are each independently selected from the group consisting of hydrogen and C₁₋₂₀ alkyl or R^{a} and R^{b} taken together with the nitrogen atom to which they are bound form an optionally substituted 3 to 10 membered heterocyclic ring. Exemplary compounds of formula II include compounds wherein R⁴ is methyl, ethyl, propyl, octyl, 2-(N,N-diethylamino)ethyl, phenyl, fluorophenyl, cyclopentyl, cyclopropyl, optionally substituted C₁₋₃ alkylene-aryl (e.g., benzyl, 2-phenethyl, 3-phenpropyl, methylbenzyl, t-butylbenzyl, fluorobenzyl, difluorobenzyl, dichlorobenzyl, nitrobenzyl, and trifluoromethylbenzyl), and optionally substituted C₁₋₃ alkylene-heteroaryl (e.g., pyridine-3-ylmethyl).

Compounds of formula II also include, but are not limited to, compounds wherein R⁵ is selected from the group consisting of optionally substituted C₁₋₂₀ alkylene-aryl, R^{d}C(═O)NR^{c}(C₁₋₂₀ alkyl), optionally substituted R^{e}R^{f}N—(C₁₋₂₀ alkylcarboxamido), optionally substituted (C₁₋₂₀ alkylcarboxamido)phenyl, optionally substituted R^{e}R^{f}N—(C₁₋₂₀ alkylamino), optionally substituted R^{e}R^{f}N—(C₁₋₂₀ alkoxy), optionally substituted C₁₋₂₀ alkylene-NR^{e}R^{f}, optionally substituted C₁₋₂₀ alkoxyphenyl, and optionally substituted C₁₋₂₀ alkylaminophenyl, wherein R^{c} is selected from the group consisting of hydrogen and C₁₋₂₀ alkyl, R^{d} is selected from the group consisting of hydrogen, C₁₋₂₀ alkyl, and optionally substituted C₁₋₂₀ aminoalkyl, and R^{e} and R^{f} are independently selected from the group consisting of hydrogen, optionally substituted C₁₋₂₀ alkyl, and optionally substituted C₁₋₂₀ aminoalkyl, or R^{e} and R^{f} taken together with the nitrogen atom to which they are bound form an optionally substituted 3 to 10 membered heterocyclic ring.

Compounds of formula II further include, but are not limited to, compounds wherein R⁵ is selected from the group consisting of optionally substituted C₁₋₃ alkylene-aryl, R^{d}C(═O)NR^{c}(C₁₋₃ alkyl), optionally substituted R^{e}R^{f}N—(C₁₋₃ alkylcarboxamido), optionally substituted (C₁₋₃ alkylcarboxamido)phenyl, optionally substituted R^{e}R^{f}N—(C₁₋₃ alkylamino), optionally substituted R^{e}R^{f}N—(C₁₋₃ alkoxy), optionally substituted C₁₋₃ alkylene-NR^{e}R^{f}, optionally substituted C₁₋₃ alkoxyphenyl, and optionally substituted C₁₋₃ alkylaminophenyl, wherein R^{c} is selected from the group consisting of hydrogen and C₁₋₃ alkyl, R^{d} is selected from the group consisting of hydrogen, C₁₋₃ alkyl, and optionally substituted C₁₋₃ aminoalkyl, and R^{e} and R^{f} are independently selected from the group consisting of hydrogen, optionally substituted C₁₋₃ alkyl, and optionally substituted C₁₋₃ aminoalkyl, or R^{e} and R^{f} taken together with the nitrogen atom to which they are bound form an optionally substituted 3 to 6 membered heterocyclic ring.

Compounds of formula II also include, but are not limited to, compounds wherein R⁵ is selected from the group consisting of optionally substituted C₁₋₃ alkylene-aryl-NR^{e}R^{f}, R^{e}R^{f}N—R^{d}C(═O)NR^{c}(C₁₋₃ alkyl), optionally substituted R^{e}R^{f}N—(C₁₋₃ alkylcarboxamido)phenyl, optionally substituted R^{e}R^{f}N—(C₁₋₃ alkoxy)phenyl, and optionally substituted R^{e}R^{f}N—(C₁₋₃ alkylamino)phenyl, wherein R^{c} is selected from the group consisting of hydrogen and C_{—31} alkyl, R^{d} is optionally substituted C₁₋₃ alkyl, and R^{e} and R^{f} are independently selected from the group consisting of hydrogen, optionally substituted C₁₋₃ alkyl, and optionally substituted C₁₋₃ aminoalkyl, or R^{e} and R^{f} taken together with the nitrogen atom to which they are bound form an optionally substituted 3 to 6 membered heterocyclic ring.

Exemplary compounds of formula II include compounds wherein R⁵ is pyridinyl, phenyl, fluorophenyl, chlorophenyl, hydroxyphenyl, methoxyphenyl, trifluoromethylphenyl, carboxyphenyl, (2-(4-aminoacetylpiperazin-1-yl)ethoxy)phenyl, (2-(4-(dimethylamino)piperidin-1-yl)ethoxy)phenyl, (2-(3-aminopyrrolidin-1-yl)ethoxy)phenyl, (3-(morpholin-4-yl)propyloxy)phenyl, (2-(piperidin-1-yl)ethoxy)phenyl, (2-(dimethylamino)ethyl)phenyl, (2-(piperidin-1-yl)ethylamino)phenyl, (2-(diethylamino)ethylcarboxamide)phenyl, (2-(4-methylpiperazin-1-yl)ethoxy)phenyl, (3-(4-methylpiperazin-1-yl)propyl)phenyl, 2-(N,N-diethylamino)ethoxy)phenyl, (2-(morpholin-4-yl)ethoxy)phenyl, benzyl, —CH₂N(CH₃)C(═O)CH₂NH₂, —CH₂N(CH₃)C(═O)CH₂CF₁₂NH₂, —CH₂N(CH₃)C(═O)CH₂N(CH₃)₂, —CH₂N(CH₃)C(═O)CH₂CH₂N(CH₃)₂, N-(2-(diethylamino)ethyl)carboxamido, N-(3-(diethylamino)propyl)carboxamido, N-(2-morpholinoethyl)carboxamido, N-(2-(piperazin-1-yl)ethyl)carboxamido, N-(3-(piperazin-1-yl)propyl)carboxamido, N-(3-morpholinopropyl)carboxamido, N-(2-(4-methylpiperazin-yl)ethyl)carboxamido, N-(3-(4-methylpiperazin-1-yl)propy))carboxamido, (2-aminoethyl)carboxamidophenyl, (3-aminopropyl)carboxamidophenyl, (3-(diethylamino)propyl)carboxamidophenyl, (2-(piperazin-1-yl)ethyl)carboxamidophenyl, (3-(piperazin-1-yl)propyl)carboxamidophenyl, (2-(diethylamino)ethyl)carboxamidophenyl, 2-(diethylamino)ethylamino, 3-(diethylamino)propylamino, 2-(dimethylamino)ethylamino, 3-(dimethylamino)propylamino, 2-aminoethylamino, 3-aminopropylamino, 2-(methylamino)ethylamino, 3-(methylamino)propylamino, 2-(2-hydroxyethylamino)ethylamino, 3-(2-hydroxyethylamino)propylamino, 2-(2-(dimethylamino)ethylamino)ethylamino, 3-(2-(dimethylamino)ethylamino)propylamino, 2-(2-(diethylamino)ethylamino)ethylamino, 3-(2-(diethylamino)ethylamino)propylamino, 2-(piperidin-1-yl)ethylamino, 3-(piperidin-1-yl)propylamino, 2-morpholinoethylamino, 3-morpholinopropylamino, 2-(piperazin-1-yl)ethylamino, 3-(piperazin-1-yl)propylamino, 2-(4-methylpiperazin-1-yl)ethylamino, 3-(4-methylpiperazin-1-yl)propylamino, 2-(diethylamino)ethoxy, 3-(diethylamino)propoxy, 2-(dimethylamino)ethoxy, 3-(dimethylamino)propoxy, 2-aminoethoxy, 3-aminopropoxy, 2-(methylamino)ethoxy, 3-(methylamino)propoxy, 2-(2-hydroxyethylamino)ethoxy, 3-(2-hydroxyethylamino)propoxy, 2-(2-(dimethylamino)ethylamino)ethoxy, 3-(2-(dimethylamino)ethylamino)propoxy, 2-(2-(diethylamino)ethylamino)ethoxy, 3-(2-(diethylamino)ethylamino)propoxy, 2-(piperidin-1-yl)ethoxy, 3-(piperidin-1-yl)propoxy, 2-morpholinoethoxy, 3-morpholinopropoxy, 2-(piperazin-1-yl)ethoxy, 3-(piperazin-1-yl)propoxy, 2-(4-methylpiperazin-1-yl)ethoxy, 3-(4-methylpiperazin-1-yl)propoxy, (2-(diethylamino)ethylamino)methyl, (3-(diethylamino)propylamino)methyl, (2-(dimethylamino)ethylamino)methyl, (3-(dimethylamino)propylamino)methyl, (2-aminoethylamino)methyl, (3-aminopropylamino)methyl, (2-(methylamino)ethylamino)methyl, (3-(methylamino)propylamino)methyl, (2-(2-hydroxyethylamino)ethylamino)methyl, (3-(2-hydroxyethylamino)propylamino)methyl, (2-(piperidin-1-yl)ethylamino)methyl, (3-(piperidin-1-yl)propylamino)methyl, (2-morpholinoethylamino)methyl, (3-morpholinopropylamino)methyl, 4-(2-aminoethoxy)phenyl, 4-(3-aminopropoxy)phenyl, 4-(2-(dimethylamino)ethoxy)phenyl, 4-(3-(dimethylamino)propoxy)phenyl, 4-(2-(Diethylamino)ethoxy)phenyl, 4-(3-(diethylamino)propoxy)phenyl, 4-(2-(2-hydroxyethylamino)ethoxy)phenyl, 4-(3-(2-hydroxyethylamino)propoxy)phenyl, 4-(2-(2-(dimethylamino)ethylamino)ethoxy)phenyl, 4-(3-(2-(dimethylamino)ethylamino)propoxy)phenyl, 4-(2-(2-(diethylamino)ethylamino)ethoxy)phenyl, 4-(3-(2-(diethylamino)ethylamino)propoxy)phenyl, 4-(2-(piperidin-1-yl)ethoxy)phenyl, 4-(3-(piperidin-1-yl)propoxy)phenyl, 4-(2-morpholinoethoxy)phenyl, 4-(3-morpholinopropoxy)phenyl, 4-(2-(piperazin-1-yl)ethoxy)phenyl, 4-(3-(piperazin-1-yl)propoxy)phenyl, 4-(2-(4-methylpiperazin-1-yl)ethoxy)phenyl, 4-(3-(4-methy)piperazin-1-yl)propoxy)phenyl, 4-(2-(4-(2-aminoacetyl)piperazin-1-yl)ethoxy)phenyl, 4-(3-(4-(2-aminoacetyl)piperazin-1-yl)propoxy)phenyl, 4-(2-(3-aminopyrrolidin-1-yl)ethoxy)phenyl, 4-(3-(3-aminopyrrolidin-1-yl)propoxy)phenyl, 4-(2-aminoethylamino)phenyl, 4-(3-aminopropylamino)phenyl, 4-(2-(dimethylamino)ethylamino)phenyl, 4-(3-(dimethylamino)propylamino)phenyl, 4-(2-(diethylamino)ethylamino)phenyl, 4-(3-(diethylamino)propylamino)phenyl, 4-(2-(2-hydroxyethylamino)ethylamino)phenyl, 4-(3-(2-hydroxyethylamino)propylamino)phenyl, 4-(2-(piperidin-1-yl)ethylamino)phenyl, 4-(3-(piperidin-1-yl)propylamino)phenyl, 4-(2-morpholinoethylamino)phenyl, 4-(3-morpholinopropylamino)phenyl, 4-(2-(pyrrolidin-1-yl)ethylamino)phenyl, and 4-(3-(pyrrolidin-1-yl)propylamino)phenyl

Compounds of formula II further include, but are not limited to, compounds wherein R⁶ is selected from the group consisting of hydrogen, C₁₋₂₀ alkyl, C₁₋₂₀ alkylene-OH, optionally substituted C₁₋₂₀ alkylene-aryl, and optionally substituted C₁₋₂₀ alkylene-heteroaryl. Exemplary compounds of formula II include compounds wherein R⁶ is methyl, ethyl, 2-(piperidin-1-yl)ethyl, and (2-aminoethyl)benzyl.

The disclosure also includes compounds having a formula II, or a pharmaceutically acceptable salt or hydrate thereof:

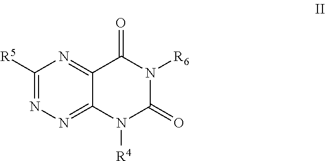

wherein R⁴ and R⁶ are independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl; and
R⁵ is selected from the group consisting of optionally substituted C₉₋₂₀ alkyl, optionally substituted C₇₋₂₀ alkenyl, optionally substituted C₂₋₂₀ alkynyl, optionally substituted C₇₋₂₀ cycloalkyl, optionally substituted C₇₋₂₀ heterocycloalkyl, optionally substituted amino(C₁₋₂₀ alkoxy)phenyl, optionally substituted amino(C₁₋₂₀ alkylamino)phenyl, optionally substituted amino(C₁₋₂₀ alkyl)carboxamidophenyl, optionally substituted amino(C₁₋₂₀ alkoxy)heteroaryl, optionally substituted amino(C₁₋₂₀ alkylamino)heteroaryl, optionally substituted amino(C₁₋₂₀ alkyl)carboxamido heteroaryl, optionally substituted amino(C₁₋₂₀ alkylamino), optionally substituted amino(C₁₋₂₀ alkoxy), optionally substituted amino(C₁₋₂₀ alkyl)carboxamido, optionally substituted amino(C₁₋₂₀ alkyl)amino(C₁₋₂₀ alkyl), and optionally substituted amino(C₁₋₂₀ alkyl)acylamino(C₁₋₂₀ alkyl).

Compounds of formula II include, but are not limited to, compounds wherein R⁴ is selected from the group consisting of hydrogen, C₁₋₂₀ alkyl, C₁₋₂₀ alkylene-OH, C₁₋₂₀ alkylene-NR^{a}R^{b}, optionally substituted C₁₋₂₀ alkylene-aryl, and optionally substituted C₁₋₂₀ alkylene-heteroaryl, wherein R^{a} and R^{b} are each independently selected from the group consisting of hydrogen and C₁₋₂₀ alkyl or R^{a} and R^{b} taken together with the nitrogen atom to which they are bound form an optionally substituted 3 to 10 membered heterocyclic ring.

Exemplary compounds of formula II include, but are not limited to, compounds wherein R⁴ is selected from the group consisting of methyl, ethyl, propyl, octyl, 2-(N,N-diethylamino)ethyl, phenyl, fluorophenyl, cyclopentyl, cyclopropyl, optionally substituted C₁₋₃ alkylene-aryl (e.g., benzyl, 2-phenethyl, 3-phenpropyl, methylbenzyl, t-butylbenzyl, fluorobenzyl, difluorobenzyl, dichlorobenzyl, nitrobenzyl, and trifluoromethylbenzyl), and optionally substituted C₁₋₃ alkylene-heteroaryl (e.g. pyridine-3-ylmethyl).

Compounds of formula II include, but are not limited to, compounds wherein R⁵ is selected from the group consisting of optionally substituted amino(C₁₋₃ alkoxy)phenyl, optionally substituted amino(C₁₋₃ alkylamino)phenyl, optionally substituted amino(C₁₋₃ alkyl)carboxamidophenyl, optionally substituted amino(C₁₋₃ alkoxy)heteroaryl, optionally substituted amino(C₁₋₃ alkylamino)heteroaryl, optionally substituted amino(C₁₋₃ alkyl)carboxamido heteroaryl, optionally substituted amino(C₁₋₃ alkylamino), optionally substituted amino(C₁₋₃ alkoxy), optionally substituted amino(C₁₋₃ alkyl)carboxamido, optionally substituted amino(C₁₋₃ alkyl)amino(C₁₋₃ alkyl), and optionally substituted amino(C₁₋₃ alkyl)acylamino(C₁₋₃ alkyl).

Compounds of formula II further include, but are not limited to, compounds wherein R⁵ is selected from the group consisting of optionally substituted R^{e}R^{d}N—(C₁₋₃ alkoxy)phenyl, optionally substituted R^{e}R^{f}N—(C₁₋₃ alkylamino)phenyl, optionally substituted R^{e}R^{f}N—(C₁₋₃ alkyl)carboxamidophenyl, optionally substituted R^{e}R^{f}N—(C₁₋₃ alkoxy)heteroaryl, optionally substituted R^{e}R^{f}N—(C₁₋₃ alkylamino)heteroaryl, optionally substituted R^{e}R^{f}N—(C₁₋₃ alkyl)carboxamido heteroaryl, optionally substituted R^{e}R^{f}N—(C₁₋₃ alkylamino), optionally substituted R^{e}R^{f}N—(C₁₋₃ alkoxy), optionally substituted R^{e}R^{f}N—(C₁₋₃ alkyl)carboxamido, optionally substituted R^{e}R^{f}N—(C₁₋₃ alkyl)amino(C₁₋₃ alkyl), and optionally substituted R^{e}R^{f}N—(C₁₋₃ alkyl)acylamino(C₁₋₁₃ alkyl);

R^{e} and R^{f} are independently selected from the group consisting of hydrogen, optionally substituted C₁₋₃ alkyl, and optionally substituted C₁₋₃ aminoalkyl; or
R^{e} and R^{f} taken together with the nitrogen atom to which they are bound form an optionally substituted 3 to 6 membered heterocyclic ring.

Compounds of formula II also include, but are not limited to, compounds wherein R⁵ is selected from the group consisting of trifluoromethylphenyl, carboxyphenyl, (2-(4-aminoacetylpiperazin-1-yl)ethoxy)phenyl, (2-(4-(dimethylamino)piperidin-1-yl)ethoxy)phenyl, (2-(3-aminopyrrolidin-1-yl)ethoxy)phenyl, (3-(morpholin-4-yl)propyloxy)phenyl, (2-(piperidin-1-yl)ethoxy)phenyl, (2-(dimethylamino)ethyl)phenyl, (2-(piperidin-1-yl)ethylamino)phenyl, (2-(diethylamino)ethylcarboxamide)phenyl, (2-(4-methylpiperazin-1-yl)ethoxy)phenyl, (3-(4-methylpiperazin-1-yl)propyl)phenyl, 2-(N,N-diethylamino)ethoxy)phenyl, (2-(morpholin-4-yl)ethoxy)phenyl, benzyl, —CH₂N(CH₃)C(═O)CH₂NH₂, CH₂N(CH₃)C(═O)CH₂CH₂NH₂, —CH₂N(CH₃)C(═O)CH₂N(CH₃)₂, —CH₂N(CH₃)C(═O)CH₂CH₂N(CH₃)₂, N-(2-(diethylamino)ethyl)carboxamido, N-(3-(diethylamino)propy))carboxamido, N-(2-morpholinoethyl)carboxamido, N-(2-(piperazin-1-yl)ethyl)carboxamido, N-(3-(piperazin-1-yl)propyl)carboxamido, N-(3-morpholinopropyl)carboxamido, N-(2-(4-methy)piperazin-1-yl)ethyl)carboxamido, N-(3-(4-methylpiperazin-1-yl)propyl)carboxamido, (2-aminoethyl)carboxamidophenyl, (3-aminopropyl)carboxamidophenyl, (3-(diethylamino)propyl)carboxyamidophenyl, (2-(piperazin-1-yl)ethyl)carboxamidophenyl, (3-(piperazin-1-yl)propyl)carboxamidophenyl, (2-(diethylamino)ethyl)carboxamidophenyl, 2-(diethylamino)ethylamino, 3-(diethylamino)propylamino, 2-(dimethylamino)ethylamino, 3-(dimethylamino)propylamino, 2-aminoethylamino, 3-aminopropylamino, 2-(methylamino)ethylamino, 3-(methylamino)propylamino, 2-(2-hydroxyethylamino)ethylamino, 3-(2-hydroxyethylamino)propylamino, 2-(2-(dimethylamino)ethylamino)ethylamino, 3-(2-(dimethylamino)ethylamino)propylamino, 2-(2-(diethylamino)ethylamino)ethylamino, 3-(2-(diethylamino)ethylamino)propylamino, 2-(piperidin-1-yl)ethylamino, 3-(piperidin-1-yl)propylamino, 2-morpholinoethylamino, 3-morpholinopropylamino, 2-(piperazin-1-yl)ethylamino, 3-(piperazin-1-yl)propylamino, 2-(4-methylpiperazin-1-yl)ethylamino, 3-(4-methylpiperazin-1-yl)propylamino, 2-(diethylamino)ethoxy, 3-(diethylamino)propoxy, 2-(dimethylamino)ethoxy, 3-(dimethylamino)propoxy, 2-aminoethoxy, 3-aminopropoxy, 2-(methylamino)ethoxy, 3-(methylamino)propoxy, 2-(2-hydroxyethylamino)ethoxy, 3-(2-hydroxyethylamino)propoxy, 2-(2-(dimethylamino)ethylamino)ethoxy, 3-(2-(dimethylamino)ethylamino)propoxy, 2-(2-(diethylamino)ethylamino)ethoxy, 3-(2-(diethylamino)ethylamino)propoxy, 2-(piperidin-1-yl)ethoxy, 3-(piperidin-1-yl)propoxy, 2-morpholinoethoxy, 3-morpholinopropoxy, 2-(piperazin-1-yl)ethoxy, 3-(piperazin-1-yl)propoxy, 2-(4-methylpiperazin-1-yl)ethoxy, 3-(4-methylpiperazin-1-yl)propoxy, (2-(diethylamino)ethylamino)methyl, (3-(diethylamino)propylamino)methyl, (2-(dimethylamino)ethylamino)methyl, (3-(dimethylamino)propylamino)methyl, (2-aminoethylamino)methyl, (3-aminopropylamino)methyl, (2-(methylamino)ethylamino)methyl, (3-(methylamino)propylamino)methyl, (2-(2-hydroxyethylamino)ethylamino)methyl, (3-(2-hydroxyethylamino)propylamino)methyl, (2-(piperidin-1-yl)ethylamino)methyl, (3-(piperidin-1-yl)propylamino)methyl. (2-morpholinoethylamino)methyl, (3-morpholinopropylamino)methyl, 4-(2-aminoethoxy)phenyl, 4-(3-aminopropoxy)phenyl, 4-(2-(dimethylamino)ethoxy)phenyl, 4-(3-(dimethylamino)propoxy)phenyl, 4-(2-(Diethylamino)ethoxy)phenyl, 4-(3-(diethylamino)propoxy)phenyl, 4-(2-(2-hydroxyethylamino)ethoxy)phenyl, 4-(3-(2-hydroxyethylamino)propoxy)phenyl, 4-(2-(2-(dimethylamino)ethylamino)ethoxy)phenyl, 4-(3-(2-(dimethylamino)ethylamino)propoxy)phenyl, 4-(2-(2-(diethylamino)ethylamino)ethoxy)phenyl, 4-(3-(2-(diethylamino)ethylamino)propoxy)phenyl, 4-(2-(piperidin-1-yl)ethoxy)phenyl, 4-(3-(piperidin-1-yl)propoxy)phenyl, 4-(2-morpholinoethoxy)phenyl, 4-(3-morpholinopropoxy)phenyl, 4-(2-(piperazin-1-yl)ethoxy)phenyl, 4-(3-(piperazin-1-yl)propoxy)phenyl, 4-(2-(4-methylpiperazin-1-yl)ethoxy)phenyl, 4-(3-(4-methy)piperazin-1-yl)propoxy)phenyl, 4-(2-(4-(2-aminoacetyl)piperazin-1-yl)ethoxy)phenyl, 4-(3-(4-(2-aminoacetyl)piperazin-1-yl)propoxy)phenyl, 4-(2-(3-aminopyrrolidin-1-yl)ethoxy)phenyl, 4-(3-(3-aminopyrrolidin-1-yl)propoxy)phenyl, 4-(2-aminoethylamino)phenyl, 4-(3-aminopropylamino)phenyl, 4-(2-(dimethylamino)ethylamino)phenyl, 4-(3-(dimethylamino)propylamino)phenyl, 4-(2-(diethylamino)ethylamino)phenyl, 4-(3-(diethylamino)propylamino)phenyl, 4-(2-(2-hydroxyethylamino)ethylamino)phenyl, 4-(3-(2-hydroxyethylamino)propylamino)phenyl, 4-(2-(piperidin-1-yl)ethylamino)phenyl, 4-(3-(piperidin-1-yl)propylamino)phenyl, 4-(2-morpholinoethylamino)phenyl, 4-(3-morpholinopropylamino)phenyl, 4-(2-(pyrrolidin-1-yl)ethylamino)phenyl, and 4-(3-(pyrrolidin-1-yl)propylamino)phenyl.

Compounds of formula II include, but are not limited to, compounds wherein R⁶ is selected from the group consisting of hydrogen, C₁₋₂₀ alkyl, C₁₋₂₀ alkylene-OH, optionally substituted C₁₋₂₀ alkylene-aryl, and optionally substituted C₁₋₂₀ alkylene-heteroaryl.

Exemplary compounds of formula II include, but are not limited to, compounds wherein R⁶ ere is selected from the group consisting of methyl, ethyl, 2-(piperidin-1-yl)ethyl, and (2-aminoethyl)benzyl.

The disclosure also includes compounds having a formula I, II, or III, or a pharmaceutically acceptable salt or hydrate thereof:

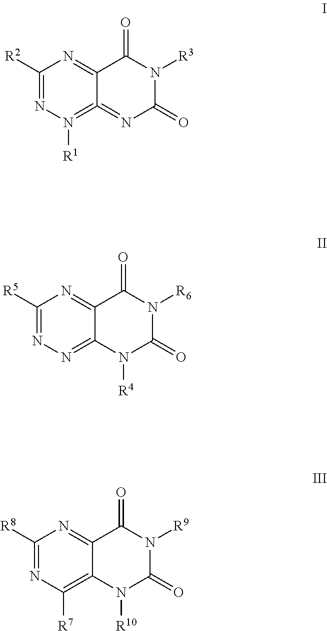

wherein R¹, R², and R³ are each independently selected from the group consisting of optionally substituted aryl and optionally substituted heteroaryl;

R⁴ is selected from the group consisting of C₇₋₂₀ alkyl, substituted C₁₋₂₀ alkyl, C₉₋₂₀ alkenyl, substituted C₂₋₂₀ alkenyl, optionally substituted C₂₋₂₀ alkynyl, C₇₋₂₀ cycloalkyl, substituted C₃₋₂₀ cycloalkyl, optionally substituted C₂₋₂₀ heterocycloalkyl, substituted aryl, and optionally substituted heteroaryl;

with the proviso that R⁴ is different from benzyl;
R⁵ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, optionally substituted heteroaryl, optionally substituted C₁₋₂₀ alkylamino, optionally substituted C₁₋₂₀ alkoxy, and optionally substituted C₁₋₂₀ alkylcarboxamido, R⁶, R⁷, R⁸, R⁹, and R¹⁰ are each independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl;
with the proviso that when R⁷ is hydrogen and R⁸ is phenyl, para-methoxyphenyl, furyl, or —CO₂Et, at least one of R⁹ and R¹⁰ is different from methyl.

The following compounds illustrate specific, nonlimiting embodiments provided by the present disclosure, and the compounds listed below are among the preferred embodiments:
3-(4-(2-(Diethylamino)ethoxy)phenyl)-1,6-dimethylpyrimido[5,4-e][1,2,4]triazine-5,7(1H,6H)-dione; 1-Benzyl-3-(4-(2-(diethylamino)ethoxy)phenyl)-6-methylpyrimido[5,4-e][1,2,4]triazine-5,7(1H,6H)-dione; 1-(4-Fluorobenzyl)-6-methyl-3-(4-(2-(4-methylpiperazin-1-yl)ethoxy)phenyl)pyrimido[5,4-e][1,2,4]triazine-5,7(1H,6H)-dione; Difluorobenzyl)-6-methyl-3-(4-(2-(morpholin-4-yl)ethoxy)phenyl)pyrimido[5,4-e][1,2,4]triazine-5,7(1H,6H)-dione; 3-(4-(2-(4-Aminoacetylpiperazin-1-yl)ethoxy)phenyl)-6-methyl-1-phenylpyrimido[5,4-e][1,2,4]triazine-5,7(1H,6H)-dione; 3-(4-(2-(4-(Dimethylamino)piperidin-1-yl)ethoxy)phenyl)-1-isopropyl-6-methylpyrimido[5,4-e][1,2,4]triazine-5,7(1H,6H)-dione; 3-(4-(2-(3-Aminopyrrolidin-1-yl)ethoxy)phenyl)-1-(3-fluorophenyl)-6-methylpyrimido[5,4-e][1,2,4]triazine-5,7(1H,6H)-dione; 1-Cyclopentyl-6-methyl-3-(4-(3-(morpholin-4-yl)propyloxy)phenyl)pyrimido[5,4-e][1,2,4]triazine-5,7(1H,6H)-dione; 1-Cyclopropyl-3-(6-(2-(diethylamino)ethoxy)pyridyl-3-yl)-6-methylpyrimido[5,4-e][1,2,4]triazine-5,7(1H,6H)-dione; 1-Cyclopentyl-3-(4-(2-(piperidin-1-yl)ethoxy)phenyl)pyrimido[5,4-e][1,2,4]triazine-5,7(1H,6H)-dione; 3-(4-(2-(Dimethylamino)ethyl)phenyl)-6-methyl-1-phenylpyrimido[5,4-e][1,2,4]triazine-5,7(1H,6H)-dione; 1-(3,4-Difluorobenzyl)-6-methyl-3-(4-(3-(4-methylpiperazin-1-yl)propyl)phenyl)pyrimido[5,4-e][1,2,4]triazine-5,7(1H,6H)-dione; 1-Isopropyl-6-methyl-3-(4-(2-(piperidin-1-yl)ethylamino)phenyl)pyrimido[5,4-d][1,2,4]triazine-5,7(1H,6H)-dione; 1-Cyclopropyl-6-(2-(piperidin-1-yl)ethyl)-3-(pyridyl-3-yl)pyrimido[5,4-e][1,2,4]triazine-5,7(1H,6H)-dione; 6-(4-(2-Aminoethyl)benzyl)-1-cyclopentyl-3-(4-fluorophenyl)pyrimido[5,4-e][1,2,4]triazine-5,7(1H,6H)-dione; 1-Cyclopentyl-3-(4-carboxyphenyl)-6-methylpyrimido[5,4-e][1,2,4]triazine-5,7(1H,6H)-dione; 3-(4-(2-(Diethylamino)ethyl)carboxamide)phenyl)-1-isopropyl-6-methylpyrimido[5,4-e][1,2,4]triazine-5,7(1H,6H)-dione;

3-(4-(2-(Diethylamino)ethoxy)phenyl)-8-(3,4-difluorobenzyl)-6-methylpyrimido[5,4-e][1,2,4]triazine-5,7(6H,8H)-dione; 8-Benzyl-3-(4-(2-(diethylamino)ethoxy)phenyl)-6-methylpyrimido[5,4-e][1,2,4]triazine-5,7(6H,8H)-dione; 8-(4-Fluorobenzyl)-6-methyl-3-(4-(2-(4-methylpiperazin-1-yl)ethoxy)phenyl)pyrimido[5,4-e][1,2,4]triazine-5,7(6H,8H)-dione; 8-(3,4-Difluorobenzyl)-6-methyl-3-(4-(2-(morpholin-4-yl)ethoxy)phenyl)pyrimido[5,4-e][1,2,4]triazine-5,7(6H,8H)-dione; 3-(4-(2-(4-Aminoacetylpiperazin-1-yl)ethoxy)phenyl)-6-methyl-8-phenylpyrimido[5,4-e][1,2,4]triazine-5,7(6H,8H)-dione; 3-(4-(2-(4-(Dimethylamino)piperidin-1-yl)ethoxy)phenyl)-8-isopropyl-6-methylpyrimido[5,4-e][1,2,4]triazine-5,7(6H,8H)-dione; 3-(4-(2-(3-Aminopyrrolidin-1-yl)ethoxy)phenyl)-8-(3-fluorophenyl)-6-methylpyrimido[5,4-e][1,2,4]triazine-5,7(6H,8H)-dione; 8-Cyclopentyl-6-methyl-3-(4-(3-(morpholin-4-yl)propyloxy)phenyl)pyrimido[5,4-e][1,2,4]triazine-5,7(6H,8H)-dione; 8-Cyclopropyl-3-(6-(2-(diethylamino)ethoxy)pyridyl-3-yl)-6-methylpyrimido[5,4-e][1,2,4]triazine-5,7(6H,8H)-dione; 8-Cyclopentyl-3-(4-(2-(piperidin-1-yl)ethoxy)phenyl)pyrimido[5,4-e][1,2,4]triazine-5,7(6H,8H)-dione; 3-(4-(2-(Dimethylamino)ethyl)phenyl)-6-methyl-8-phenylpyrimido[5,4-e][1,2,4]triazine-5,7(6H,8H)-dione; 8-(3,4-Difluorobenzyl)-6-methyl-3-(4-(3-(4-methylpiperazin-1-yl)propyl)phenyl)pyrimido[5,4-e][1,2,4]triazine-5,7(6H,8H)-dione; 8-Isopropyl-6-methyl-3-(4-(24 piperidin-1-yl)ethylamino)phenyl)pyrimido[5,4-e][1,2,4]triazine-5,7(6H,8H)-dione; 8-Cyclopropyl-6-(2-(piperidin-1-yl)ethyl)-3-(pyridyl-3-yl)pyrimido[5,4-e][1,2,4]triazine-5,7(6H,8H)-dione; 6-(4-(2-Aminoethyl)benzyl)-8-cyclopentyl-3-(4-fluorophenyl)pyrimido[5,4-e][1,2,4]triazine-5,7(6H,8H)-dione; 8-Cyclopentyl-3-(4-carboxyphenyl)-6-methylpyrimido[5,4-e][1,2,4]triazine-5,7(6H,8H)-dione; 3-(4-(2-(Diethylamino)ethylcarboxamide)phenyl)-6,8-dimethylpyrimido[5,4-e][1,2,4]triazine-5,7(6H,8H)-dione; 6,8-Dimethyl-3-(4-(2-(4-methylpiperazin-1-yl)ethoxy)phenyl)pyrimido[5,4-e][1,2,4]triazine-5,7(6H,8H)-dione; 8-Methyl-3-(4-(2-(4-methylpiperazin-1-yl)ethoxy)phenyl)pyrimido[5,4-e][1,2,4]triazine-5,7(6H,8H)-dione; 3-(4-(2-(Diethylamino)ethoxy)phenyl)-6,8-dimethylpyrimido[5,4-e][1,2,4]triazine-5,7(6H,8H)-dione; 3-(4-(2-(Diethylamino)ethoxy)phenyl)-8-methylpyrimido[5,4-e][1,2,4]triazine-5,7(6H,8H)-dione; 6,8-Dimethyl-3-(4-(2-(morpholin-4-yl)ethoxy)phenyl)pyrimido[5,4-e][1,2,4]triazine-5,7(6H,8H)-dione; 3-(2-(Diethylamino)ethylamino)-6,8-dimethylpyrimido[5,4-e][1,2,4]triazine-5,7(6H,8H)-dione; 3-(3-(Diethylamino)propylamino)-6,8-dimethylpyrimido[5,4-e][1,2,4]triazine-5,7(6H,8H)-dione; 3-(2-(Dimethylamino)ethylamino)-6,8-dimethylpyrimido[5,4-e][1,2,4]triazine-5,7(6H,8H)-dione; 3-(3-(Dimethylamino)propylamino)-6,8-dimethylpyrimido[5,4-e][1,2,4]triazine-5,7(6H,8H)-dione; 3-(2-Aminoethylamino)-6,8-dimethylpyrimido[5,4-e][1,2,4-]triazine-5,7(6H,8H)-dione; 3-(3-Aminopropylamino)-6,8-dimethylpyrimido[5,4-e][1,2,4]triazine-5,7(6H,8H)-dione; 6,8-Dimethyl-3-(2-(methylamino)ethylamino)pyrimido[5,4-e][1,2,4]triazine-5,7(6H,8H)-dione; 6,8-Dimethyl-3-(3-(methylamino)propylamino)pyrimido[5,4-e][1,2,4]triazine-5,7(6H,8H)-dione; 3-(2-(2-Hydroxyethylamino)ethylamino)-6,8-dimethylpyrimido[5,4-e][1,2,4]triazine-5,7(6H,8H)-dione; 3-(3-(2-Hydroxyethylamino)propylamino)-6,8-dimethylpyrimido[5,4-e][1,2,4]triazine-5,7(6H,8H)-dione; 3-(2-(2-(Dimethylamino)ethylamino)ethylamino)-6,8-dimethylpyrimido[5,4-e][1,2,4]triazine-5,7(6H,8H)-dione; 34342-(Dimethylamino)ethylamino)propylamino)-6,8-dimethylpyrimido[5,4-e][1,2,4]triazine-5,7(6H,8H)-dione; 3-(2-(2-(Diethylamino)ethylamino)ethylamino)-6,8-dimethylpyrimido[5,4-e][1,2,4]triazine-5,7(6H,8H)-dione; 34342-(Diethylamino)ethylamino)propylamino)-6,8-dimethylpyrimido[5,4-e][1,2,4]triazine-5,7(6H,8H)-dione; 6,8-Dimethyl-3-(2-(piperidin-1-yl)ethylamino)pyrimido[5,4-e][1,2,4]triazine-5,7(6H,8H)-dione; 6,8-Dimethyl-3-(3-(piperidin-1-yl)propylamino)pyrimido[5,4-e][1,2,4]triazine-5,7(6H,8H)-dione; 6,8-Dimethyl-3-(2-morpholinoethylamino)pyrimido[5,4-e][1,2,4]triazine-5,7(6H,8H)-dione; 6,8-dimethyl-3-(3-morpholinopropyl)amino)pyrimido[5,4-e][1,2,4]triazine-5,7(6H,8H)-dione; 6,8-Dimethyl-3-(2-(piperazin-1-yl)ethylamino)pyrimido[5,4-e][1,2,4]triazine-5,7(6H,8H)-dione; 6,8-Dimethyl-3-(3-(piperazin-1-yl)propylamino)pyrimido[5,4-e][1,2,4]triazine-5,7(6H,8H)-dione; 6,8-Dimethyl-3-(2-(4-methylpiperazin-1-yl)ethylamino)pyrimido[5,4-e][1,2,4]triazine-5,7(6H,8H)-dione; 6,8-Dimethyl-3-(3-(4-methylpiperazin-1-yl)propylamino)pyrimido[5,4-e][1,2,4]triazine-5,7(6H,8H)-dione; 3-(2-(Diethylamino)ethoxy)-6,8-dimethylpyrimido[5,4-e][1,2,4]triazine-5,7(6H,8H)-dione; 3-(3-(Diethylamino)propoxy)-6,8-dimethylpyrimido[5,4-e][1,2,4]triazine-5,7(6H,8H)-dione; 3-(2-(Dimethylamino)ethoxy)-6,8-dimethylpyrimido[5,4-e][1,2,4]triazine-5,7(6H,8H)-dione; 3-(3-(Dimethylamino)propoxy)-6,8-dimethylpyrimido[5,4-e][1,2,4]triazine-5,7(6H,8H)-dione; 3-(2-Aminoethoxy)-6,8-dimethylpyrimido[5,4-e][1,2,4]triazine-5,7(6H,8H)-dione; 3-(3-Aminopropoxy)-6,8-dimethylpyrimido[5,4-e][1,2,4]triazine-5,7(6H,8H)-dione; 6,8-Dimethyl-3-(2-(methylamino)ethoxy)pyrimido[5,4-e][1,2,4]triazine-5,7(6H,8H)-dione; 6,8-Dimethyl-3-(3-(methylamino)propoxy)pyrimido[5,4-e][1,2,4]triazine-5,7(6H,8H)-dione; 3-(2-(2-Hydroxyethylamino)ethoxy)-6,8-dimethylpyrimido[5,4-e][1,2,4]triazine-5,7(6H,8H)-dione; 3-(3-(2-Hydroxyethylamino)propoxy)-6,8-dimethylpyrimido[5,4-e][1,2,4]triazine-5,7(6H,8H)-dione; 3-(2-(2-(Dimethylamino)ethylamino)ethoxy)-6,8-dimethylpyrimido[5,4-e][1,2,4]triazine-5,7(6H,8H)-dione; 3-(3-(2-(Dimethylamino)ethylamino)propoxy)-6,8-dimethylpyrimido[5,4-e][1,2,4]triazine-5,7(6H,8H)-dione; 3-(2-(2-(Diethylamino)ethylamino)ethoxy)-6,8-dimethylpyrimido[5,4-e][1,2,4]triazine-5,7(6H,8H)-dione; 3-(3-(2-(Diethylamino)ethylamino)propoxy)-6,8-dimethylpyrimido[5,4-e][1,2,4]triazine-5,7(6H,8H)-dione; 6,8-Dimethyl-3-(2-(piperidin-1-yl)ethoxy)pyrimido[5,4-e][1,2,4]triazine-5,7(6H,8H)-dione; 6,8-Dimethyl-3-(3-(piperidin-1-yl)propoxy)pyrimido[5,4-e][1,2,4]triazine-5,7(6H,8H)-dione; 6,8-Dimethyl-3-(2-morpholinoethoxy)pyrimido[5,4-e][1,2,4]triazine-5,7(6H,8H)-dione; 6,8-Dimethyl-3-(3-morpholinopropoxy)pyrimido[5,4-e][1,2,4]triazine-5,7(6H,8H)-dione; 6,8-Dimethyl-3-(2-(piperazin-1-yl)ethoxy)pyrimido[5,4-e][1,2,4]triazine-5,7(6H,8H)-dione; 6,8-Dimethyl-3-(3-(piperazin-1-yl)propoxy)pyrimido[5,4-d][1,2,4]triazine-5,7(6H,8H)-dione; 6,8-Dimethyl-3-(2-(4-methylpiperazin-1-yl)ethoxy)pyrimido[5,4-e][1,2,4]triazine-5,7(6H,8H)-dione; 6,8-Dimethyl-3-(3-(4-methylpiperazin-1-yl)propoxy)pyrimido[5,4-e][1,2,4]triazine-5,7(6H,8H)-dione; 3-((2-(Diethylamino)ethylamino)methyl)-6,8-dimethylpyrimido[5,4-e][1,2,4]triazine-5,7(6H,8H)-dione; 3-((3-(Diethylamino)propylamino)methyl)-6,8-dimethylpyrimido[5,4-e][1,2,4]triazine-5,7(6H,8H)-dione; 3-((2-(Dimethylamino)ethylamino)methyl)-6,8-dimethylpyrimido[5,4-e][1,2,4]triazine-5,7(6H,8H)-dione; 3-((3-(Dimethylamino)propylamino)methyl)-6,8-dimethylpyrimido[5,4-e][1,2,4]triazine-5,7(6H,8H)-dione; 3-((2-Aminoethylamino)methyl)-6,8-dimethylpyrimido[5,4-e][1,2,4]triazine-5,7(6H,8H)-dione; 3-((3-Aminopropylamino)methyl)-6,8-dimethylpyrimido[5,4-e][1,2,4]triazine-5,7(6H,8H)-dione; 6,8-Dimethyl-3-((2-(methylamino)ethylamino)methyl)pyrimido[5,4-e][1,2,4]triazine-5,7(6H,8H)-dione; 6,8-Dimethyl-3-((3-(methylamino)propylamino)methyl)pyrimido[5,4-e][1,2,4]triazine-5,7(6H,8H)-dione; 3-((2-(2-Hydroxyethylamino)ethylamino)methyl)-6,8-dimethylpyrimido[5,4-e][1,2,4]triazine-5,7(6H,8H)-dione; 3-((3-(2-Hydroxyethylamino)propylamino)methyl)-6,8-dimethylpyrimido[5,4-e][1,2,4]triazine-5,7(6H,8H)-dione; 2-Amino-N-((6,8-dimethyl-5,7-dioxo-5,6,7,8-tetrahydropyrimido[5,4-e][1,2,4]triazin-3-yl)methyl)-N-methylacetamide; 3-Amino-N-((6,8-dimethyl-5,7-dioxo-5,6,7,8-tetrahydropyrimido[5,4-e][1,2,4]triazin-3-yl)methyl)-N-methylpropanamide; N-((6,8-Dimethyl-5,7-dioxo-5,6,7,8-tetrahydropyrimido[5,4-e][1,2,4]triazin-3-yl)methyl)-2-(dimethylamino)-N-methylacetamide; N-((6,8-Dimethyl-5,7-dioxo-5,6,7,8-tetrahydropyrimido[5,4-e][1,2,4]triazin-3-yl)methyl)-3-(dimethylamino)-N-methylpropanamide; 6,8-Dimethyl-3-((2-(piperidin-1-yl)ethylamino)methyl)pyrimido[5,4-e][1,2,4]triazine-5,7(6H,8H)-dione; 6,8-Dimethyl-3-((3-(piperidin-1-yl)propylamino)methyl)pyrimido[5,4-e][1,2,4]triazine-5,7(6H,8H)-dione; 6,8-Dimethyl-3-((2-morpholinoethylamino)methyl)pyrimido[5,4-e][1,2,4]triazine-5,7(6H,8H)-dione; 6,8-Dimethyl-3-((3-morpholinopropylamino)methyl)pyrimido[5,4-e][1,2,4]triazine-5,7(6H,8H)-dione; N-(2-(Diethylamino)ethyl)-6,8-dimethyl-5,7-dioxo-5,6,7,8-tetrahydropyrimido[5,4-e][1,2,4]triazine-3-carboxamide; N-(3-(Diethylamino)propyl)-6,8-dimethyl-5,7-dioxo-5,6,7,8-tetrahydropyrimido[5,4-e][1,2,4]triazine-3-carboxamide; 6,8-Dimethyl-N-(2-morpholinoethyl)-5,7-dioxo-5,6,7,8-tetrahydropyrimido[5,4-e][1,2,4]triazine-3-carboxamide; 6,8-Dimethyl-5,7-dioxo-N-(2-(piperazin-1-yl)ethyl)-5,6,7,8-tetrahydropyrimido[5,4-e][1,2,4]triazine-3-carboxamide; 6,8-Dimethyl-5,7-dioxo-N-(3-(piperazin-1-yl)propyl)-5,6,7,8-tetrahydropyrimido[5,4-e][1,2,4]triazine-3-carboxamide; 6,8-Dimethyl-N-(3-morpholinopropyl)-5,7-dioxo-5,6,7,8-tetrahydropyrimido[5,4-e][1,2,4]triazine-3-carboxamide; 6,8-Dimethyl-N-(2-(4-methylpiperazin-1-yl)ethyl)-5,7-dioxo-5,6,7,8-tetrahydropyrimido[5,4-e][1,2,4]triazine-3-carboxamide; 6,8-Dimethyl-N-(3-(4-methylpiperazin-1-yl)propyl)-5,7-dioxo-5,6,7,8-tetrahydropyrimido[5,4-e][1,2,4]triazine-3-carboxamide; 8-Cyclopentyl-3-(2-(diethylamino)ethylamino)-6-methylpyrimido[5,4-e][1,2,4]triazine-5,7(6H,8H)-dione; 8-Cyclopentyl-3-(3-(diethylamino)propylamino)-6-methylpyrimido[5,4-e][1,2,4]triazine-5,7(6H,8H)-dione; 8-Cyclopentyl-3-(2-(dimethylamino)ethylamino)-6-methylpyrimido[5,4-e][1,2,4]triazine-5,7(6H,8H)-dione; 8-Cyclopentyl-3-(3-(dimethylamino)propylamino)-6-methylpyrimido[5,4-e][1,2,4]triazine-5,7(6H,8H)-dione; 3-(2-Aminoethylamino)-8-cyclopentyl-6-methylpyrimido[5,4-e][1,2,4]triazine-5,7(6H,8H)-dione; 3-(3-Aminopropylamino)-8-cyclopentyl-6-methylpyrimido[5,4-e][1,2,4]triazine-5,7(6H,8H)-dione; 8-Cyclopentyl-6-methyl-3-(2-(methylamino)ethylamino)pyrimido[5,4-e][1,2,4]triazine-5,7(6H,8H)-dione; 8-Cyclopentyl-6-methyl-3-(3-(methylamino)propylamino)pyrimido[5,4-e][1,2,4]triazine-5,7(6H,8H)-dione; 8-Cyclopentyl-3-(2-(2-hydroxyethylamino)ethylamino)-6-methylpyrimido[5,4-e][1,2,4]triazine-5,7(6H,8H)-dione; 8-Cyclopentyl-3-(3-(2-hydroxyethylamino)propylamino)-6-methylpyrimido[5,4-e][1,2,4]triazine-5,7(6H,8H)-dione; 8-Cyclopentyl-3-(2-(2-(dimethylamino)ethylamino)ethylamino)-6-methylpyrimido[5,4-e][1,2,4]triazine-5,7(6H,8H)-dione; 8-Cyclopentyl-3-(3-(2-(dimethylamino)ethylamino)propylamino)-6-methylpyrimido[5,4-e][1,2,4]triazine-5,7(6H,8H)-dione; 8-Cyclopentyl-3-(2-(2-(diethylamino)ethylamino)ethylamino)-6-methylpyrimido[5,4-e][1,2,4]triazine-5,7(6H,8H)-dione; 8-Cyclopentyl-3-(3-(2-(diethylamino)ethylamino)propylamino)-6-methylpyrimido[5,4-e][1,2,4]triazine-5,7(6H,8H)-dione; 8-Cyclopentyl-6-methyl-3-(2-(piperidin-1-yl)ethylamino)pyrimido[5,4-e][1,2,4]triazine-5,7(6H,8H)-dione; 8-Cyclopentyl-6-methyl-3-(3-(piperidin-1-yl)propylamino)pyrimido[5,4-e][1,2,4]triazine-5,7(6H,8H)-dione; 8-Cyclopentyl-6-methyl-3-(2-morpholinoethylamino)pyrimido[5,4-e][1,2,4]triazine-5,7(6H,8H)-dione; 8-Cyclopentyl-6-methyl-3-(3-morpholinopropylamino)pyrimido[5,4-e][1,2,4]triazine-5,7(6H,8H)-dione; 8-Cyclopentyl-6-methyl-3-(2-(piperazin-1-yl)ethylamino)pyrimido[5,4-e][1,2,4]triazine-5,7(6H,8H)-dione; 8-Cyclopentyl-6-methyl-3-(3-(piperazin-1-yl)propylamino)pyrimido[5,4-e][1,2,4]triazine-5,7(6H,8H)-dione; 8-Cyclopentyl-6-methyl-3-(2-(4-methylpiperazin-1-yl)ethylamino)pyrimido[5,4-e][1,2,4]triazine-5,7(6H,8H)-dione; 8-Cyclopentyl-6-methyl-3-(3-(4-methylpiperazin-1-yl)propylamino)pyrimido[5,4-e][1,2,4]triazine-5,7(6H,8H)-dione; 8-Cyclopentyl-3-(2-(diethylamino)ethoxy)-6-methylpyrimido[5,4-e][1,2,4]triazine-5,7(6H,8H)-dione; 8-Cyclopentyl-3-(3-(diethylamino)propoxy)-6-methylpyrimido[5,4-e][1,2,4]triazine-5,7(6H,8H)-dione; 8-Cyclopentyl-3-(2-(dimethylamino)ethoxy)-6-methylpyrimido[5,4-e][1,2,4]triazine-5,7(6H,8H)-dione; 8-Cyclopentyl-3-(3-(dimethylamino)propoxy)-6-methylpyrimido[5,4-e][1,2,4]triazine-5,7(6H,8H)-dione; 3-(2-Aminoethoxy)-8-cyclopentyl-6-methylpyrimido[5,4-e][1,2,4]triazine-5,7(6H,8H)-dione; 3-(3-Aminopropoxy)-8-cyclopentyl-6-methylpyrimido[5,4-e][1,2,4]triazine-5,7(6H,8H)-dione; 8-Cyclopentyl-6-methyl-3-(2-(methylamino)ethoxy)pyrimido[5,4-e][1,2,4]triazine-5,7(6H,8H)-dione; 8-Cyclopentyl-6-methyl-3-(3-(methylamino)propoxy)pyrimido[5,4-e][1,2,4]triazine-5,7(6H,8H)-dione; 8-Cyclopentyl-3-(2-(2-hydroxyethylamino)ethoxy)-6-methylpyrimido[5,4-e][1,2,4]triazine-5,7(6H,8H)-dione; 8-Cyclopentyl-3-(3-(2-hydroxyethylamino)propoxy)-6-methylpyrimido[5,4-e][1,2,4]triazine-5,7(6H,8H)-dione; 8-Cyclopentyl-3-(2-(2-(dimethylamino)ethylamino)ethoxy)-6-methylpyrimido[5,4-e][1,2,4]triazine-5,7(6H,8H)-dione; 8-Cyclopentyl-3-(3-(2-(dimethylamino)ethylamino)propoxy)-6-methylpyrimido[5,4-e][1,2,4]triazine-5,7(6H,8H)-dione; 8-Cyclopentyl-3-(2-(2-(diethylamino)ethylamino)ethoxy)-6-methylpyrimido[5,4-e][1,2,4]triazine-5,7(6H,8H)-dione; 8-Cyclopentyl-3-(3-(2-(diethylamino)ethylamino)propoxy)-6-methylpyrimido[5,4-e][1,2,4]triazine-5,7(6H,8H)-dione; 8-Cyclopentyl-6-methyl-3-(2-(piperidin-1-yl)ethoxy)pyrimido[5,4-e][1,2,4]triazine-5,7(6H,8H)-dione; 8-Cyclopentyl-6-methyl-3-(3-(piperidin-1-yl)propoxy)pyrimido[5,4-e][1,2,4]triazine-5,7(6H,8H)-dione; 8-Cyclopentyl-6-methyl-3-(2-morpholinoethoxy)pyrimido[5,4-e][1,2,4]triazine-5,7(6H,8H)-dione; 8-Cyclopentyl-6-methyl-3-(3-morpholinopropoxy)pyrimido[5,4-e][1,2,4]triazine-5,7(6H,8H)-dione; 8-Cyclopentyl-6-methyl-3-(2-(piperazin-1-yl)ethoxy)pyrimido[5,4-e][1,2,4]triazine-5,7(6H,8H)-dione; 8-Cyclopentyl-6-methyl-3-(3-(piperazin-1-yl)propoxy)pyrimido[5,4-e][1,2,4]triazine-5,7(6H,8H)-dione; 8-Cyclopentyl-6-methyl-3-(2-(4-methylpiperazin-1-yl)ethoxy)pyrimido[5,4-e][1,2,4]triazine-5,7(6H,8H)-dione; 8-Cyclopentyl-6-methyl-3-(3-(4-methylpiperazin-1-yl)propoxy)pyrimido[5,4-e][1,2,4]triazine-5,7(6H,8H)-dione; 8-Cyclopentyl-3-((2-(diethylamino)ethylamino)methyl)-6-methylpyrimido[5,4-e][1,2,4]triazine-5,7(6H,8H)-dione; 8-Cyclopentyl-3-((3-(diethylamino)propylamino)methyl)-6-methylpyrimido[5,4-e][1,2,4]triazine-5,7(6H,8H)-dione; 8-Cyclopentyl-3-((2-(dimethylamino)ethylamino)methyl)-6-methylpyrimido[5,4-e][1,2,4]triazine-5,7(6H,8H)-dione; 8-Cyclopentyl-3-((3-(dimethylamino)propylamino)methyl)-6-methylpyrimido[5,4-e][1,2,4]triazine-5,7(6H,8H)-dione; 3-((2-Aminoethylamino)methyl)-8-cyclopentyl-6-methylpyrimido[5,4-e][1,2,4]triazine-5,7(6H,8H)-dione; 3-((3-Aminopropylamino)methyl)-8-cyclopentyl-6-methylpyrimido[5,4-e][1,2,4]triazine-5,7(6H,8H)-dione; 8-Cyclopentyl-6-methyl-3-((2-(methylamino)ethylamino)methyl)pyrimido[5,4-e][1,2,4]triazine-5,7(6H,8H)-dione; 8-Cyclopentyl-6-methyl-3-((3-(methylamino)propylamino)methyl)pyrimido[5,4-e][1,2,4]triazine-5,7(6H,8H)-dione; 8-Cyclopentyl-3-((2-(2-hydroxyethylamino)ethylamino)methyl)-6-methylpyrimido[5,4-e][1,2,4]triazine-5,7(6H,8H)-dione; 8-Cyclopentyl-3-((3-(2-hydroxyethylamino)propylamino)methyl)-6-methylpyrimido[5,4-e][1,2,4]triazine-5,7(6H,8H)-dione; 2-Amino-N-((8-cyclopentyl-6-methyl-5,7-dioxo-5,6,7,8-tetrahydropyrimido[5,4-e][1,2,4]triazin-3-yl)methyl)-N-methylacetamide; 3-Amino-N-((8-cyclopentyl-6-methyl-5,7-dioxo-5,6,7,8-tetrahydropyrimido[5,4-e][1,2,4]triazin-3-yl)methyl)-N-methylpropanamide; N-((8-Cyclopentyl-6-methyl-5,7-dioxo-5,6,7,8-tetrahydropyrimido[5,4-e][1,2,4]yl)methyl)-2-(dimethylamino)-N-methylacetamide; N-((8-Cyclopentyl-6-methyl-5,7-dioxo-5,6,7,8-tetrahydropyrimido[5,4-e][1,2,4]triazin-3-yl)methyl)-3-(dimethylamino)-N-methylpropanamide; 8-Cyclopentyl-6-methyl-3-(2-(piperidin-1-yl)ethylamino)methyl)pyrimido[5,4-e][1,2,4]triazine-5,7(6H,8H)-dione; 8-Cyclopentyl-6-methyl-3-((3-(piperidin-1-yl)propylamino)methyl)pyrimido[5,4-e][1,2,4]triazine-5,7(6H,8H)-dione; 8-Cyclopentyl-6-methyl-3-(2-morpholinoethylamino)methyl)pyrimido[5,4-e][1,2,4]triazine-5,7(6H,8H)-dione; 8-Cyclopentyl-6-methyl-3-((3-morpholinopropylamino)methyl)pyrimido[5,4-e][1,2,4]triazine-5,7(6H,8H)-dione; 8-Cyclopentyl-N-(2-(diethylamino)ethyl)-6-methyl-5,7-dioxo-5,6,7,8-tetrahydropyrimido[5,4-e][1,2,4]triazine-3-carboxamide; 8-Cyclopentyl-N-(3-(diethylamino)propyl)-6-methyl-5,7-dioxo-5,6,7,8-tetrahydropyrimido[5,4-e][1,2,4]triazine-3-carboxamide; 8-Cyclopentyl-6-methyl-N-(2-morpholinoethyl)-5,7-dioxo-5,6,7,8-tetrahydropyrimido[5,4-e][1,2,4]triazine-3-carboxamide; 8-Cyclopentyl-6-methyl-N-(3-morpholinopropyl)-5,7-dioxo-5,6,7,8-tetrahydropyrimido[5,4-d][1,2,4]triazine-3-carboxamide; 8-Cyclopentyl-6-methyl-5,7-dioxo-N-(2-(piperazin-1-yl)ethyl)-5,6,7,8-tetrahydropyrimido[5,4-e][1,2,4]triazine-3-carboxamide; 8-Cyclopentyl-6-methyl-5,7-dioxo-N-(3-(piperazin-1-yl)propyl)-5,6,7,8-tetrahydropyrimido[5,4-e][1,2,4]triazine-3-carboxamide; 8-Cyclopentyl-6-methyl-N-(2-(4-methylpiperazin-1-yl)ethyl)-5,7-dioxo-5,6,7,8-tetrahydropyrimido[5,4-e][1,2,4]triazine-3-carboxamide; 8-Cyclopentyl-6-methyl-N-(3-(4-methylpiperazin-1-yl)propyl)-5,7-dioxo-5,6,7,8-tetrahydropyrimido[5,4-e][1,2,4]triazine-3-carboxamide; 3-(2-(Diethylamino)ethylamino)-8-(4-fluorophenyl)-6-methylpyrimido[5,4-e][1,2,4]triazine-5,7(6H,8H)-dione; 3-(3-(Diethylamino)propylamino)-8-(4-fluorophenyl)-6-methylpyrimido[5,4-e][1,2,4]triazine-5,7(6H,8H)-dione; 3-(2-(Dimethylamino)ethylamino)-8-(4-fluorophenyl)-6-methylpyrimido[5,4-e][1,2,4]triazine-5,7(6H,8H)-dione; 3-(3-(Dimethylamino)propylamino)-8-(4-fluorophenyl)-6-methylpyrimido[5,4-e][1,2,4]triazine-5,7(6H,8H)-dione; 3-(2-Aminoethylamino)-8-(4-fluorophenyl)-6-methylpyrimido[5,4-e][1,2,4]triazine-5,7(6H,8H)-dione; 3-(3-Aminopropylamino)-8-(4-fluorophenyl)-6-methylpyrimido[5,4-e][1,2,4]triazine-5,7(6H,8H)-dione; 8-(4-Fluorophenyl)-6-methyl-3-(2-(methylamino)ethylamino)pyrimido[5,4-e][1,2,4]triazine-5,7(6H,8H)-dione; 8-(4-Fluorophenyl)-6-methyl-3-(3-(methylamino)propylamino)pyrimido[5,4-e][1,2,4]triazine-5,7(6H,8H)-dione; 8-(4-Fluorophenyl)-3(2-(2-hydroxyethylamino)ethylamino)-6-methylpyrimido[5,4-e][1,2,4]triazine-5,7(6H,8H)-dione; 8-(4-Fluorophenyl)-3-(3-(2-hydroxyethylamino)propylamino)-6-methylpyrimido[5,4-e][1,2,4]triazine-5,7(6H,8H)-dione; 3-(2-(2-(Dimethylamino)ethylamino)ethylamino)-8-(4-fluorophenyl)-6-methylpyrimido[5,4-e][1,2,4]triazine-5,7(6H,8H)-dione; 3-(3-(2-(Dimethylamino)ethylamino)propylamino)-8-(4-fluorophenyl)-6-methy)pyrimido[5,4-e][1,2,4]triazine-5,7(6H,8H)-dione; 3-(2-(2-(Diethylamino)ethylamino)ethylamino)-8-(4-fluorophenyl)-6-methylpyrimido[5,4-e][1,2,4]triazine-5,7(6H,8H)-dione; 3-(3-(2-(Diethylamino)ethylamino)propylamino)-8-(4-fluorophenyl)-6-methylpyrimido[5,4-e][1,2,4]triazine-5,7(6H,8H)-dione; 8-(4-Fluorophenyl)-6-methyl-3-(2-(piperidin-1-yl)ethylamino)pyrimido[5,4-e][1,2,4]triazine-5,7(6H,8H)-dione;
8-(4-Fluorophenyl)-6-methyl-3-(3-(piperidin-1-yl)propylamino)pyrimido[5,4-e][1,2,4]triazine-5,7(6H,8H)-dione; 8-(4-Fluorophenyl)-6-methyl-3-(2-morpholinoethylamino)pyrimido[5,4-e][1,2,4]triazine-5,7(6H,8H)-dione; 8-(4-Fluorophenyl)-6-methyl-3-(3-morpholinopropylamino)pyrimido[5,4-e][1,2,4]triazine-5,7(6H,8H)-dione; 8-(4-Fluorophenyl)-6-methyl-3-(2-(piperazin-1-yl)ethylamino)pyrimido[5,4-e][1,2,4]triazine-5,7(6H,8H)-dione; 8-(4-Fluorophenyl)-6-methyl-3-(3-(piperazin-1-yl)propylamino)pyrimido[5,4-e][1,2,4]triazine-5,7(6H,8H)-dione; 8-(4-Fluorophenyl)-6-methyl-3-(2-(4-methylpiperazin-1-yl)ethylamino)pyrimido[5,4-e][1,2,4]triazine-5,7(6H,8H)-dione; 8-(4-Fluorophenyl)-6-methyl-3-(3-(4-methylpiperazin-1-yl)propylamino)pyrimido[5,4-e][1,2,4]triazine-5,7(6H,8H)-dione; 3-(2-(Diethylamino)ethoxy)-8-(4-fluorophenyl)-6-methylpyrimido[5,4-e][1,2,4]triazine-5,7(6H,8H)-dione; 3-(3-(Diethylamino)propoxy)-8-(4-fluorophenyl)-6-methylpyrimido[5,4-e][1,2,4]triazine-5,7(6H,8H)-dione; 3-(2-(Dimethylamino)ethoxy)-8-(4-fluorophenyl)-6-methylpyrimido[5,4-e][1,2,4]triazine-5,7(6H,8H)-dione; 3-(3-(Dimethylamino)propoxy)-8-(4-fluorophenyl)-6-methylpyrimido[5,4-e][1,2,4]triazine-5,7(6H,8H)-dione; 3-(2-Aminoethoxy)-8-(4-fluorophenyl)-6-methylpyrimido[5,4-e][1,2,4]triazine-5,7(6H,8H)-dione; 3-(3-Aminopropoxy)-8-(4-fluorophenyl)-6-methylpyrimido[5,4-e][1,2,4]triazine-5,7(6H,8H)-dione; 8-(4-Fluorophenyl)-6-methyl-3-(2-(methylamino)ethoxy)pyrimido[5,4-e][1,2,4]triazine-5,7(6H,8H)-dione; 8-(4-Fluorophenyl)-6-methyl-3-(3-(methylamino)propoxy)pyrimido[5,4-d][1,2,4]triazine-5,7(6H,8H)-dione; 8-(4-Fluorophenyl)-3-(2-(2-hydroxyethylamino)ethoxy)-6-methylpyrimido[5,4-e][1,2,4]triazine-5,7(6H,8H)-dione; 8-(4-Fluorophenyl)-3-(3-(2-hydroxyethylamino)propoxy)-6-methylpyrimido[5,4-e][1,2,4]triazine-5,7(6H,8H)-dione; 3-(2-(2-(Dimethylamino)ethylamino)ethoxy)-8-(4-fluorophenyl)-6-methylpyrimido[5,4-e][1,2,4]triazine-5,7(6H,8H)-dione; 3-(3-(2-(Dimethylamino)ethylamino)propoxy)-8-(4-fluorophenyl)-6-methylpyrimido[5,4-e][1,2,4]triazine-5,7(6H,8H)-dione; 3-(2-(2-(Diethylamino)ethylamino)ethoxy)-8-(4-fluorophenyl)-6-methylpyrimido[5,4-e][1,2,4]triazine-5,7(6H,8H)-dione; 3-(3-(2-(Diethylamino)ethylamino)propoxy)-8-(4-fluorophenyl)-6-methylpyrimido[5,4-e][1,2,4]triazine-5,7(6H,8H)-dione; 8-(4-Fluorophenyl)-6-methyl-3-(2-(piperidin-1-yl)ethoxy)pyrimido[5,4-e][1,2,4]triazine-5,7(6H,8H)-dione; 8-(4-Fluorophenyl)-6-methyl-3-(3-(piperidin-1-yl)propoxy)pyrimido[5,4-e][1,2,4]triazine-5,7(6H,8H)-dione; 8-(4-Fluorophenyl)-6-methyl-3-(2-morpholinoethoxy)pyrimido[5,4-e][1,2,4]triazine-5,7(6H,8H)-dione; 8-(4-Fluorophenyl)-6-methyl-3-(3-morpholinopropoxy)pyrimido[5,4-e][1,2,4]triazine-5,7(6H,8H)-dione; 8-(4-Fluorophenyl)-6-methyl-3-(2-(piperazin-1-yl)ethoxy)pyrimido[5,4-e][1,2,4]triazine-5,7(6H,8H)-dione; 8-(4-Fluorophenyl)-6-methyl-3-(3-(piperazin-1-yl)propoxy)pyrimido[5,4-e][1,2,4]triazine-5,7(6H,8H)-dione; 8-(4-Fluorophenyl)-6-methyl-3-(2-(4-methylpiperazin-1-yl)ethoxy)pyrimido[5,4-e][1,2,4]triazine-5,7(6H,8H)-dione; 8-(4-Fluorophenyl)-6-methyl-3-(3-(4-methylpiperazin-1-yl)propoxy)pyrimido[5,4-e][1,2,4]triazine-5,7(6H,8H)-dione; 3-((2-(Diethylamino)ethylamino)methyl)-8-(4-fluorophenyl)-6-methylpyrimido[5,4-e][1,2,4]triazine-5,7(6H,8H)-dione; 3-((3-(Diethylamino)propylamino)methyl)-8-(4-fluorophenyl)-6-methylpyrimido[5,4-e][1,2,4]triazine-5,7(6H,8H)-dione; 3-((2-(Dimethylamino)ethylamino)methyl)-8-(4-fluorophenyl)-6-methylpyrimido[5,4-e][1,2,4]triazine-5,7(6H,8H)-dione; 3-((3-(Dimethylamino)propylamino)methyl)-8-(4-fluorophenyl)-6-methylpyrimido[5,4-e][1,2,4]triazine-5,7(6H,8H)-dione; 3-((2-Aminoethylamino)methyl)-8-(4-fluorophenyl)-6-methylpyrimido[5,4-e][1,2,4]triazine-5,7(6H,8H)-dione; 3-((3-Aminopropylamino)methyl)-8-(4-fluorophenyl)-6-methylpyrimido[5,4-e][1,2,4]triazine-5,7(6H,8H)-dione; 8-(4-Fluorophenyl)-6-methyl-3-((2-(methylamino)ethylamino)methyl)pyrimido[5,4-e][1,2,4]triazine-5,7(6H,8H)-dione; 8-(4-Fluorophenyl)-6-methyl-3-((3-(methylamino)propylamino)methy)pyrimido[5,4-e][1,2,4]triazine-5,7(6H,8H)-dione; 8-(4-Fluorophenyl)-3-((2-(2-hydroxyethylamino)ethylamino)methyl)-6-methylpyrimido[5,4-e][1,2,4]triazine-5,7(6H,8H)-dione; 8-(4-Fluorophenyl)-3-((3-(2-hydroxyethylamino)propylamino)methyl)-6-methylpyrimido[5,4-e][1,2,4]triazine-5,7(6H,8H)-dione; 2-Amino-N-((8-(4-fluorophenyl)-6-methyl-5,7-dioxo-5,6,7,8-tetrahydropyrimido[5,4-e][1,2,4]triazin-3-yl)methyl)-N-methylacetamide; 3-Amino-N-((8-(4-fluorophenyl)-6-methyl-5,7-dioxo-5,6,7,8-tetrahydropyrimido[5,4-e][1,2,4]triazin-3-yl)methyl)-N-methylpropanamide; 2-(Dimethylamino)-N-((8-(4-fluorophenyl)-6-methyl-5,7-dioxo-5,6,7,8-tetrahydropyrimido[5,4-e][1,2,4]triazin-3-yl)methyl)-N-methylacetamide; 3-(Dimethylamino)-N-((8-(4-fluorophenyl)-6-methyl-5,7-dioxo-5,6,7,8-tetrahydropyrimido[5,4-e][1,2,4]triazin-3-yl)methyl)-N-methylpropanamide; 8-(4-Fluorophenyl)-6-methyl-3-((2-(piperidin-1-yl)ethylamino)methyl)pyrimido[5,4-e][1,2,4]triazine-5,7(6H,8H)-dione; 8-(4-Fluorophenyl)-6-methyl-3-((3-(piperidin-1-yl)propylamino)methyl)pyrimido[5,4-e][1,2,4]triazine-5,7(6H,8H)-dione; 8-(4-Fluorophenyl)-6-methyl-3-((2-morpholinoethylamino)methyl)pyrimido[5,4-e][1,2,4]triazine-5,7(6H,8H)-dione; 8-(4-Fluorophenyl)-6-methyl-3-((3-morpholinopropylamino)methyl)pyrimido[5,4-e][1,2,4]triazine-5,7(6H,8H)-dione; N-(2-(Diethylamino)ethyl)-8-(4-fluorophenyl)-6-methyl-5,7-dioxo-5,6,7,8-tetrahydropyrimido[5,4-e][1,2,4]triazine-3-carboxamide; N-(3-(Diethylamino)propyl)-8-(4-fluorophenyl)-6-methyl-5,7-dioxo-5,6,7,8-tetrahydropyrimido[5,4-e][1,2,4]triazine-3-carboxamide; 8-(4-Fluorophenyl)-6-methyl-N-(2-morpholinoethyl)-5,7-dioxo-5,6,7,8-tetrahydropyrimido[5,4-e][1,2,4]triazine-3-carboxamide; 8-(4-Fluorophenyl)-6-methyl-N-(3-morpholinopropyl)-5,7-dioxo-5,6,7,8-tetrahydropyrimido[5,4-e][1,2,4]triazine-3-carboxamide; 8-(4-Fluorophenyl)-6-methyl-5,7-dioxo-N-(2-(piperazin-1-yl)ethyl)-5,6,7,8-tetrahydropyrimido[5,4-e][1,2,4]triazine-3-carboxamide; 8-(4-Fluorophenyl)-6-methyl-5,7-dioxo-N-(3-(piperazin-1-yl)propyl)-5,6,7,8-tetrahydropyrimido[5,4-e][1,2,4]triazine-3-carboxamide; 8-(4-Fluorophenyl)-6-methyl-N-(2-(4-methylpiperazin-1-yl)ethyl)-5,7-dioxo-5,6,7,8-tetrahydropyrimido[5,4-e][1,2,4]triazine-3-carboxamide; 8-(4-Fluorophenyl)-6-methyl-N-(3-(4-methylpiperazin-1-yl)propyl)-5,7-dioxo-5,6,7,8-tetrahydropyrimido[5,4-e][1,2,4]triazine-3-carboxamide; 3-(2-(Diethylamino)ethylamino)-8-(3,4-difluorobenzyl)-6-methylpyrimido[5,4-e][1,2,4]triazine-5,7(6H,8H)-dione; 3-(3-(Diethylamino)propylamino)-8-(3,4-difluorobenzyl)-6-methylpyrimido[5,4-e][1,2,4]triazine-5,7(6H,8H)-dione; Difluorobenzyl)-3-(2-(dimethylamino)ethylamino)-6-methylpyrimido[5,4-e][1,2,4]triazine-5,7(6H,8H)-dione; 8-(3,4-Difluorobenzyl)-3-(3-(dimethylamino)propylamino)-6-methylpyrimido[5,4-e][1,2,4]triazine-5,7(6H,8H)-dione; 3-(2-Aminoethylamino)-8-(3,4-difluorobenzyl)-6-methylpyrimido[5,4-e][1,2,4]triazine-5,7(6H,8H)-dione; 3-(3-Aminopropylamino)-8-(3,4-difluorobenzyl)-6-methylpyrimido[5,4-e][1,2,4]triazine-5,7(6H,8H)-dione; 8-(3,4-Difluorobenzyl)-6-methyl-3-(2-(methylamino)ethylamino)pyrimido[5,4-e][1,2,4]triazine-5,7(6H,8H)-dione; 8-(3,4-Difluorobenzyl)-6-methyl-3-(3-(methylamino)propylamino)pyrimido[5,4-e][1,2,4]triazine-5,7(6H,8H)-dione; 8-(3,4-Difluorobenzyl)-3-(2-(2-hydroxyethylamino)ethylamino)-6-methylpyrimido[5,4-e][1,2,4]triazine-5,7(6H,8H)-dione; 8-(3,4-Difluorobenzyl)-3-(3-(2-hydroxyethylamino)propylamino)-6-methylpyrimido[5,4-e][1,2,4]triazine-5,7(6H,8H)-dione; 8-(3,4-Difluorobenzyl)-3-(2-(2-(dimethylamino)ethylamino)ethylamino)-6-methylpyrimido[5,4-e][1,2,4]triazine-5,7(6H,8H)-dione; 8-(3,4-Difluorobenzyl)-3-(3-(2-(dimethylamino)ethylamino)propylamino)-6-methylpyrimido[5,4-e][1,2,4]triazine-5,7(6H,8H)-dione; 3-(2-(2-(Diethylamino)ethylamino)ethylamino)-8-(3,4-difluorobenzyl)-6-methylpyrimido[5,4-e][1,2,4]triazine-5,7(6H,8H)-dione; 34342-(Diethylamino)ethylamino)propylamino)-8-(3,4-difluorobenzyl)-6-methylpyrimido[5,4-e][1,2,4]triazine-5,7(6H,8H)-dione; 8-(3,4-Difluorobenzyl)-6-methyl-3-(2-(piperidin-1-yl)ethylamino)pyrimido[5,4-e][1,2,4]triazine-5,7(6H,8H)-dione; 8-(3,4-Difluorobenzyl)-6-methyl-3-(3-(piperidin-1-yl)propylamino)pyrimido[5,4-e][1,2,4]triazine-5,7(6H,8H)-dione; 8-(3,4-Difluorobenzyl)-6-methyl-3-(2-morpholinoethylamino)pyrimido[5,4-e][1,2,4]triazine-5,7(6H,8H)-dione; 8-(3,4-Difluorobenzyl)-6-methyl-3-(3-morpholinopropylamino)pyrimido[5,4-e][1,2,4]triazine-5,7(6H,8H)-dione; Difluorobenzyl)-6-methyl-3-(2-(piperazin-1-yl)ethylamino)pyrimido[5,4-e][1,2,4]triazine-5,7(6H,8H)-dione; 8-(3,4-Difluorobenzyl)-6-methyl-3-(3-(piperazin-1-yl)propylamino)pyrimido[5,4-e][1,2,4]triazine-5,7(6H,8H)-dione; 8-(3,4-Difluorobenzyl)-6-methyl-3-(2-(4-methylpiperazin-1-yl)ethylamino)pyrimido[5,4-e][1,2,4]triazine-5,7(6H,8H)-dione; 8-(3,4-Difluorobenzyl)-6-methyl-3-(3-(4-methylpiperazin-1-yl)propylamino)pyrimido[5,4-e][1,2,4]triazine-5,7(6H,8H)-dione; 3-(2-(Diethylamino)ethoxy)-8-(3,4-difluorobenzyl)-6-methylpyrimido[5,4-e][1,2,4]triazine-5,7(6H,8H)-dione; 3-(3-(Diethylamino)propoxy)-8-(3,4-difluorobenzyl)-6-methylpyrimido[5,4-e][1,2,4]triazine-5,7(6H,8H)-dione; 8-(3,4-Difluorobenzyl)-3-(2-(dimethylamino)ethoxy)-6-methylpyrimido[5,4-e][1,2,4]triazine-5,7(6H,8H)-dione; Difluorobenzyl)-3-(3-(dimethylamino)propoxy)-6-methylpyrimido[5,4-e][1,2,4]triazine-5,7(6H,8H)-dione; 3-(2-Aminoethoxy)-8-(3,4-difluorobenzyl)-6-methylpyrimido[5,4-e][1,2,4]triazine-5,7(6H,8H)-dione; 3-(3-Aminopropoxy)-8-(3,4-difluorobenzyl)-6-methylpyrimido[5,4-e][1,2,4]triazine-5,7(6H,8H)-dione; 8-(3,4-Difluorobenzyl)-6-methyl-3-(2-(methylamino)ethoxy)pyrimido[5,4-e][1,2,4]triazine-5,7(6H,8H)-dione; Difluorobenzyl)-6-methyl-3-(3-(methylamino)propoxy)pyrimido[5,4-e][1,2,4]triazine-5,7(6H,8H)-dione; 8-(3,4-Difluorobenzyl)-3-(2-(2-hydroxyethylamino)ethoxy)-6-methylpyrimido[5,4-e][1,2,4]triazine-5,7(6H,8H)-dione; 8-(3,4-Difluorobenzyl)-3-(3-(2-hydroxyethylamino)propoxy)-6-methylpyrimido[5,4-e][1,2,4]triazine-5,7(6H,8H)-dione; 8-(3,4-Difluorobenzyl)-3-(2-(2-(dimethylamino)ethylamino)ethoxy)-6-methylpyrimido[5,4-e][1,2,4]triazine-5,7(6H,8H)-dione; 8-(3,4-Difluorobenzyl)-3-(3-(2-(dimethylamino)ethylamino)propoxy)-6-methylpyrimido[5,4-e][1,2,4]triazine-5,7(6H,8H)-dione; 3-(2-(2-(Diethylamino)ethylamino)ethoxy)-8(3,4-difluorobenzyl)-6-methylpyrimido[5,4-e][1,2,4]triazine-5,7(6H,8H)-dione; 3-(3-(2-(Diethylamino)ethylamino)propoxy)-8-(3,4-difluorobenzyl)-6-methylpyrimido[5,4-e][1,2,4]triazine-5,7(6H,8H)-dione; 8-(3,4-Difluorobenzyl)-6-methyl-3-(2-(piperidin-1-yl)ethoxy)pyrimido[5,4-e][1,2,4]triazine-5,7(6H,8H)-dione; 8-(3,4-Difluorobenzyl)-6-methyl-3-(3-(piperidin-1-yl)propoxy)pyrimido[5,4-e][1,2,4]triazine-5,7(6H,8H)-dione; 8-(3,4-Difluorobenzyl)-6-methyl-3-(2-morpholinoethoxy)pyrimido[5,4-e][1,2,4]triazine-5,7(6H,8H)-dione; 8-(3,4-Difluorobenzyl)-6-methyl-3-(3-morpholinopropoxy)pyrimido[5,4-e][1,2,4]triazine-5,7(6H,8H)-dione; 8-(3,4-Difluorobenzyl)-6-methyl-3-(2-(piperazin-1-yl)ethoxy)pyrimido[5,4-e][1,2,4]triazine-5,7(6H,8H)-dione; 8-(3,4-Difluorobenzyl)-6-methyl-3-(3-(piperazin-1-yl)propoxy)pyrimido[5,4-e][1,2,4]triazine-5,7(6H,8H)-dione; 8-(3,4-Difluorobenzyl)-6-methyl-3-(2-(4-methylpiperazin-1-yl)ethoxy)pyrimido[5,4-e][1,2,4]triazine-5,7(6H,8H)-dione; 8-(3,4-Difluorobenzyl)-6-methyl-3-(3-(4-methylpiperazin-1-yl)propoxy)pyrimido[5,4-e][1,2,4]triazine-5,7(6H,8H)-dione; 3-((2-(Diethylamino)ethylamino)methyl)-8-(3,4-difluorobenzyl)-6-methylpyrimido[5,4-e][1,2,4]triazine-5,7(6H,8H)-dione; 3-((3-(Diethylamino)propylamino)methyl)-8-(3,4-difluorobenzyl)-6-methylpyrimido[5,4-e][1,2,4]triazine-5,7(6H,8H)-dione; 8-(3,4-Difluorobenzyl)-3-((2-(dimethylamino)ethylamino)methyl)-6-methylpyrimido[5,4-e][1,2,4]triazine-5,7(6H,8H)-dione; 8-(3,4-Difluorobenzyl)-3-((3-(dimethylamino)propylamino)methyl)-6-methylpyrimido[5,4-d][1,2,4]triazine-5,7(6H,8H-dione; 3-((2-Aminoethylamino)methyl)-8-(3,4-difluorobenzyl)-6-methylpyrimido[5,4-e][1,2,4]triazine-5,7(6H,8H)-dione; 3-((3-Aminopropylamino)methyl)-8-(3,4-difluorobenzyl)-6-methylpyrimido[5,4-e][1,2,4]triazine-5,7(6H,8H)-dione; Difluorobenzyl)-6-methyl-3-((2-(methylamino)ethylamino)methyl)pyrimido[5,4-e][1,2,4]triazine-5,7(6H,8H)-dione; 8-(3,4-Difluorobenzyl)-6-methyl-3-((3-(methylamino)propylamino)methyl)pyrimido[5,4-e][1,2,4]triazine-5,7(6H,8H)-dione; Difluorobenzyl)-3-((2-(2-hydroxyethylamino)ethylamino)methyl)-6-methylpyrimido[5,4-e][1,2,4]triazine-5,7(6H,8H)-dione; 8-(3,4-Difluorobenzyl)-3-((3-(2-hydroxyethylamino)propylamino)methyl)-6-methylpyrimido[5,4-e][1,2,4]triazine-5,7(6H,8H)-dione; 2-Amino-N-((8-(3,4-difluorobenzyl)-6-methyl-5,7-dioxo-5,6,7,8-tetrahydropyrimido[5,4-e][1,2,4]triazin-3-yl)methyl)-N-methylacetamide; 3-Amino-N-((8-(3,4-difluorobenzyl)-6-methyl-5,7-dioxo-5,6,7,8-tetrahydropyrimido[5,4-e][1,2,4]triazin-3-yl)methyl)-N-methylpropanamide; N-((8-(3,4-Difluorobenzyl)-6-methyl-5,7-dioxo-5,6,7,8-tetrahydropyrimido[5,4-e][1,2,4]triazin-3-yl)methyl)-2-(dimethylamino)-N-methylacetamide; N-((8-(3,4-Difluorobenzyl)-6-methyl-5,7-dioxo-5,6,7,8-tetrahydropyrimido[5,4-e][1,2,4]triazin-3-yl)methyl)-3-(dimethylamino)-N-methylpropanamide; Difluorobenzyl)-6-methyl-3-((2-(piperidin-1-yl)ethylamino)methyl)pyrimido[5,4-e][1,2,4]triazine-5,7(6H,8H)-dione; 8-(3,4-Difluorobenzyl)-6-methyl-3-((3-(piperidin-1-yl)propylamino)methyl)pyrimido[5,4-e][1,2,4]triazine-5,7(6H,8H)-dione; Difluorobenzyl)-6-methyl-3-((2-morpholinoethylamino)methyl)pyrimido[5,4-e][1,2,4]triazine-5,7(6H,8H)-dione; 8-(3,4-Difluorobenzyl)-6-methyl-3-((3-morpholinopropylamino)methyl)pyrimido[5,4-e][1,2,4]triazine-5,7(6H,8H)-dione; N-(2-(Diethylamino)ethyl)-8-(3,4-difluorobenzyl)-6-methyl-5,7-dioxo-5,6,7,8-tetrahydropyrimido[5,4-e][1,2,4]triazine-3-carboxamide; N-(3-(Diethylamino)propyl)-8-(3,4-difluorobenzyl)-6-methyl-5,7-dioxo-5,6,7,8-tetrahydropyrimido[5,4-e][1,2,4]triazine-3-carboxamide; 8-(3,4-Difluorobenzyl)-6-methyl-N-(2-morpholinoethyl)-5,7-dioxo-5,6,7,8-tetrahydropyrimido[5,4-e][1,2,4]triazine-3-carboxamide; 8-(3,4-Difluorobenzyl)-6-methyl-N-(3-morpholinopropyl)-5,7-dioxo-5,6,7,8-tetrahydropyrimido[5,4-e][1,2,4]triazine-3-carboxamide; 8-(3,4-Difluorobenzyl)-6-methyl-5,7-dioxo-N-(2-(piperazin-1-yl)ethyl)-5,6,7,8-tetrahydropyrimido[5,4-e][1,2,4]triazine-3-carboxamide; 8-(3,4-Difluorobenzyl)-6-methyl-5,7-dioxo-N-(3-(piperazin-1-yl)propyl)-5,6,7,8-tetrahydropyrimido[5,4-e][1,2,4]triazine-3-carboxamide; 8-(3,4-Difluorobenzyl)-6-methyl-N-(2-(4-methylpiperazin-1-yl)ethyl)-5,7-dioxo-5,6,7,8-tetrahydropyrimido[5,4-e][1,2,4]triazine-3-carboxamide; Difluorobenzyl)-6-methyl-N-(3-(4-methylpiperazin-1-yl)propyl)-5,7-dioxo-5,6,7,8-tetrahydropyrimido[5,4-e][1,2,4]triazine-3-carboxamide; 3-(2-(Diethylamino)ethylamino)-6-methyl-8-(pyridin-3-ylmethyl)pyrimido[5,4-e][1,2,4]triazine-5,7(6H,8H)-dione; 3-(3-(Diethylamino)propylamino)-6-methyl-8-(pyridin-3-ylmethyl)pyrimido[5,4-e][1,2,4]triazine-5,7(6H,8H)-dione; 3-(2-(Dimethylamino)ethylamino)-6-methyl-8-(pyridin-3-ylmethyl)pyrimido[5,4-e][1,2,4]triazine-5,7(6H,8H)-dione; 3-(3-(Dimethylamino)propylamino)-6-methyl-8-(pyridin-3-ylmethyl)pyrimido[5,4-e][1,2,4]triazine-5,7(6H,8H)-dione; 3-(2-Aminoethylamino)-6-methyl-8-(pyridin-3-ylmethyl)pyrimido[5,4-e][1,2,4]triazine-5,7(6H,8H)-dione; 3-(3-Aminopropylamino)-6-methyl-8-(pyridin-3-ylmethyl)pyrimido[5,4-e][1,2,4]triazine-5,7(6H,8H)-dione; 6-Methyl-3-(2-(methylamino)ethylamino)-8-(pyridin-3-ylmethyl)pyrimido[5,4-e][1,2,4]triazine-5,7(6H,8H)-dione; 6-Methyl-3-(3-(methylamino)propylamino)-8-(pyridin-3-ylmethyl)pyrimido[5,4-e][1,2,4]triazine-5,7(6H,8H)-dione; 3-(2-(2-Hydroxyethylamino)ethylamino)-6-methyl-8-(pyridin-3-ylmethyl)pyrimido[5,4-e][1,2,4]triazine-5,7(6H,8H)-dione; 3-(3-(2-Hydroxyethylamino)propylamino)-6-methyl-8-(pyridin-3-ylmethyl)pyrimido[5,4-e][1,2,4]triazine-5,7(6H,8H)-dione; 3-(2-(2-(Dimethylamino)ethylamino)ethylamino)-6-methyl-8-(pyridin-3-ylmethyl)pyrimido[5,4-e][1,2,4]triazine-5,7(6H,8H)-dione; 3-(3-(2-(Dimethylamino)ethylamino)propylamino)-6-methyl-8-(pyridin-3-ylmethyl)pyrimido[5,4-e][1,2,4]triazine-5,7(6H,8H)-dione; 3-(2-(2-(Diethylamino)ethylamino)ethylamino)-6-methyl-8-(pyridin-3-ylmethyl)pyrimido[5,4-e][1,2,4]triazine-5,7(6H,8H)-dione; 3-(3-(2-(Diethylamino)ethylamino)propylamino)-6-methyl-8-(pyridin-3-ylmethyl)pyrimido[5,4-e][1,2,4]triazine-5,7(6H,8H)-dione; 6-Methyl-3-(2-(piperidin-1-yl)ethylamino)-8-(pyridin-3-ylmethyl)pyrimido[5,4-e][1,2,4]triazine-5,7(6H,8H)-dione; 6-Methyl-3-(3-(piperidin-1-yl)propylamino)-8-(pyridin-3-ylmethyl)pyrimido[5,4-e][1,2,4]triazine-5,7(6H,8H)-dione; 6-Methyl-3-(2-morpholinoethylamino)-8-(pyridin-3-ylmethyl)pyrimido[5,4-e][1,2,4]triazine-5,7(6H,8H)-dione; 6-Methyl-3-(3-morpholinopropylamino)-8-(pyridin-3-ylmethyl)pyrimido[5,4-e][1,2,4]triazine-5,7(6H,8H)-dione; 6-Methyl-3-(2-(piperazin-1-yl)ethylamino)-8-(pyridin-3-ylmethyl)pyrimido[5,4-e][1,2,4]triazine-5,7(6H,8H)-dione; 6-Methyl-3-(3-(piperazin-1-yl)propylamino)-8-(pyridin-3-ylmethyl)pyrimido[5,4-e][1,2,4]triazine-5,7(6H,8H)-dione; 6-Methyl-3-(2-(4-methylpiperazin-1-yl)ethylamino)-8-(pyridin-3-ylmethyl)pyrimido[5,4-e][1,2,4]triazine-5,7(6H,8H)-dione; 6-Methyl-3-(3-(4-methylpiperazin-1-yl)propylamino)-8-(pyridin-3-ylmethyl)pyrimido[5,4-e][1,2,4]triazine-5,7(6H,8H)-dione; 3-(2-(Diethylamino)ethoxy)-6-methyl-8-(pyridin-3-ylmethyl)pyrimido[5,4-e][1,2,4]triazine-5,7(6H,8H)-dione; 3-(3-(Diethylamino)propoxy)-6-methyl-8-(pyridin-3-ylmethyl)pyrimido[5,4-e][1,2,4]triazine-5,7(6H,8H)-dione; 3-(2-(Dimethylamino)ethoxy)-6-methyl-8-(pyridin-3-ylmethyl)pyrimido[5,4-e][1,2,4]triazine-5,7(6H,8H)-dione; 3-(3-(Dimethylamino)propoxy)-6-methyl-8-(pyridin-3-ylmethyl)pyrimido[5,4-e][1,2,4]triazine-5,7(6H,8H)-dione; 3-(2-Aminoethoxy)-6-methyl-8-(pyridin-3-ylmethyl)pyrimido[5,4-e][1,2,4]triazine-5,7(6H,8H)-dione; 3-(3-Aminopropoxy)-6-methyl-8-(pyridin-3-ylmethyl)pyrimido[5,4-e][1,2,4]triazine-5,7(6H,8H)-dione; 6-Methyl-3-(2-(methylamino)ethoxy)-8-(pyridin-3-ylmethyl)pyrimido[5,4-e][1,2,4]triazine-5,7(6H,8H)-dione; 6-Methyl-3-(3-(methylamino)propoxy)-8-(pyridin-3-ylmethyl)pyrimido[5,4-e][1,2,4]triazine-5,7(6H,8H)-dione; 3-(2-(2-Hydroxyethylamino)ethoxy)-6-methyl-8-(pyridin-3-ylmethyl)pyrimido[5,4-e][1,2,4]triazine-5,7(6H,8H)-dione; 34342-Hydroxyethylamino)propoxy)-6-methyl-8-(pyridin-3-ylmethyl)pyrimido[5,4-e][1,2,4]triazine-5,7(6H,8H-dione; 3-(2-(2-(Dimethylamino)ethylamino)ethoxy)-6-methyl-8-(pyridin-3-ylmethyl)pyrimido[5,4-e][1,2,4]triazine-5,7(6H,8H)-dione; 3-(3-(2-(Dimethylamino)ethylamino)propoxy)-6-methyl-8-(pyridin-3-ylmethyl)pyrimido[5,4-e][1,2,4]triazine-5,7(6H,8H)-dione; 3-(2-(2-(Diethylamino)ethylamino)ethoxy)-6-methyl-8-(pyridin-3-ylmethyl)pyrimido[5,4-e][1,2,4]triazine-5,7(6H,8H)-dione; 3-(3-(2-(Diethyl amino)ethylamino)propoxy)-6-methyl-8-(pyridin-3-ylmethyl)pyrimido[5,4-e][1,2,4]triazine-5,7(6H,8H)-dione; 6-Methyl-3-(2-(piperidin-1-yl)ethoxy)-8-(pyridin-3-ylmethyl)pyrimido[5,4-e][1,2,4]triazine-5,7(6H,8H)-dione; 6-Methyl-3-(3-(piperidin-1-yl)propoxy)-8-(pyridin-3-ylmethyl)pyrimido[5,4-e][1,2,4]triazine-5,7(6H,8H)-dione; 6-Methyl-3-(2-morpholinoethoxy)-8-(pyridin-3-ylmethyl)pyrimido[5,4-e][1,2,4]triazine-5,7(6H,8H)-dione; 6-Methyl-3-(3-morpholinopropoxy)-8-(pyridin-3-ylmethyl)pyrimido[5,4-e][1,2,4]triazine-5,7(6H,8H)-dione; 6-Methyl-3-(2-(piperazin-1-yl)ethoxy)-8-(pyridin-3-ylmethyl)pyrimido[5,4-e][1,2,4]triazine-5,7(6H,8H)-dione; 6-Methyl-3-(3-(piperazin-1-yl)propoxy)-8-(pyridin-3-ylmethyl)pyrimido[5,4-e][1,2,4]triazine-5,7(6H,8H)-dione; 6-Methyl-3-(2-(4-methylpiperazin-1-yl)ethoxy)-8-(pyridin-3-ylmethyl)pyrimido[5,4-e][1,2,4]triazine-5,7(6H,8H)-dione; 6-Methyl-3-(3-(4-methylpiperazin-1-yl)propoxy)-8-(pyridin-3-ylmethyl)pyrimido[5,4-e][1,2,4]triazine-5,7(6H,8H)-dione; 3-((2-(Diethylamino)ethylamino)methyl)-6-methyl-8-(pyridin-3-ylmethyl)pyrimido[5,4-e][1,2,4]triazine-5,7(6H,8H)-dione; 3-((3-(Diethylamino)propylamino)methyl)-6-methyl-8-(pyridin-3-ylmethyl)pyrimido[5,4-e][1,2,4]triazine-5,7(6H,8H)-dione; 3-((2-(Dimethylamino)ethylamino)methyl)-6-methyl-8-(pyridin-3-ylmethyl)pyrimido[5,4-e][1,2,4]triazine-5,7(6H,8H)-dione; 3-((3-(Dimethylamino)propylamino)methyl)-6-methyl-8-(pyridin-3-ylmethyl)pyrimido[5,4-e][1,2,4]triazine-5,7(6H,8H)-dione; 3-((2-Aminoethylamino)methyl)-6-methyl-8-(pyridin-3-ylmethyl)pyrimido[5,4-e][1,2,4]triazine-5,7(6H,8H)-dione; 3-((3-Aminopropylamino)methyl)-6-methyl-8-(pyridin-3-ylmethyl)pyrimido[5,4-e][1,2,4]triazine-5,7(6H,8H)-dione; 6-Methyl-3-((2-(methylamino)ethylamino)methyl)-8-(pyridin-3-ylmethyl)pyrimido[5,4-e][1,2,4]triazine-5,7(6H,8H)-dione; 6-Methyl-3-((3-(methylamino)propylamino)methyl)-8-(pyridin-3-ylmethyl)pyrimido[5,4-e][1,2,4]triazine-5,7(6H,8H)-dione; 3-((2-(2-Hydroxyethylamino)ethylamino)methyl)-6-methyl-8-(pyridin-3-ylmethyl)pyrimido[5,4-e][1,2,4]triazine-5,7(6H,8H)-dione; 3-((3-(2-Hydroxyethylamino)propylamino)methyl)-6-methyl-8-(pyridin-3-ylmethyl)pyrimido[5,4-e][1,2,4]triazine-5,7(6H,8H)-dione; 2-Amino-N-methyl-N-((6-methyl-5,7-dioxo-8-(pyridin-3-ylmethyl)-5,6,7,8-tetrahydropyrimido[5,4-e][1,2,4]triazin-3-yl)methyl)acetamide; 3-Amino-N-methyl-N-((6-methyl-5,7-dioxo-8-(pyridin-3-ylmethyl)-5,6,7,8-tetrahydropyrimido[5,4-e][1,2,4]triazin-3-yl)methyl)propanamide; 2-(Dimethylamino)-N-methyl-N-((6-methyl-5,7-dioxo-8-(pyridin-3-ylmethyl)-5,6,7,8-tetrahydropyrimido[5,4-e][1,2,4]triazin-3-yl)methyl)acetamide; 3-(Dimethylamino)-N-methyl-N-((6-methyl-5,7-dioxo-8-(pyridin-3-ylmethyl)-5,6,7,8-tetrahydropyrimido[5,4-e][1,2,4]triazin-3-yl)methyl)propanamide; 6-Methyl-3-((2-(piperidin-1-yl)ethylamino)methyl)-8-(pyridin-3-ylmethyl)pyrimido[5,4-e][1,2,4]triazine-5,7(6H,8H)-dione; 6-Methyl-3-((3-(piperidin-1-yl)propylamino)methyl)-8-(pyridin-3-ylmethyl)pyrimido[5,4-e][1,2,4]triazine-5,7(6H,8H)-dione; 6-Methyl-3-((2-morpholinoethylamino)methyl)-8-(pyridin-3-ylmethyl)pyrimido[5,4-e][1,2,4]triazine-5,7(6H,8H)-dione; 6-Methyl-3-((3-morpholinopropylamino)methyl)-8-(pyridin-3-ylmethyl)pyrimido[5,4-e][1,2,4]triazine-5,7(6H,8H)-dione; N-(2-(Diethylamino)ethyl)-6-methyl-5,7-dioxo-8-(pyridin-3-ylmethyl)-5,6,7,8-tetrahydropyrimido[5,4-][1,2,4]triazine-3-carboxamide; N-(3-(Diethylamino)propyl)-6-methyl-5,7-dioxo-8-(pyridin-3-ylmethyl)-5,6,7,8-tetrahydropyrimido[5,4-e][1,2,4]triazine-3-carboxamide; 6-Methyl-N-(2-morpholinoethyl)-5,7-dioxo-8-(pyridin-3-ylmethyl)-5,6,7,8-tetrahydropyrimido[5,4-e][1,2,4]triazine-3-carboxamide; 6-Methyl-N-(3-morpholinopropyl)-5,7-dioxo-8-(pyridin-3-ylmethyl)-5,6,7,8-tetrahydropyrimido[5,4-e][1,2,4]triazine-3-carboxamide; 6-Methyl-N-(2-(4-methylpiperazin-1-yl)ethyl)-5,7-dioxo-8-(pyridin-3-ylmethyl)-5,6,7,8-tetrahydropyrimido[5,4-e][1,2,4]triazine-3-carboxamide; 6-Methyl-N-(3-(4-methylpiperazin-1-yl)propyl)-5,7-dioxo-8-(pyridin-3-ylmethyl)-5,6,7,8-tetrahydropyrimido[5,4-e][1,2,4]triazine-3-carboxamide; 6-Methyl-5,7-dioxo-N-(2-(piperazin-1-yl)ethyl)-8-(pyridin-3-ylmethyl)-5,6,7,8-tetrahydropyrimido[5,4-e][1,2,4]triazine-3-carboxamide; 6-Methyl-5,7-dioxo-N-(3-(piperazin-1-yl)propyl)-8-(pyridin-3-ylmethyl)-5,6,7,8-tetrahydropyrimido[5,4-e][1,2,4]triazine-3-carboxamide; 3-(4-(2-Aminoethoxy)phenyl)-6,8-dimethylpyrimido[5,4-e][1,2,4]triazine-5,7(6H,8H)-dione; 3-(4-(3-Aminopropoxy)phenyl)-6,8-dimethylpyrimido[5,4-e][1,2,4]triazine-5,7(6H,8H)-dione; 3-(4-(2-(Dimethylamino)ethoxy)phenyl)-6,8-dimethylpyrimido[5,4-e][1,2,4]triazine-5,7(6H,8H)-dione; 3-(4(3-(Dimethylamino)propoxy)phenyl)-6,8-dimethylpyrimido[5,4-e][1,2,4]triazine-5,7(6H,8H)-dione; 3-(4-(3-(Diethylamino)propoxy)phenyl)-6,8-dimethylpyrimido[5,4-e][1,2,4]triazine-5,7(6H,8H)-dione; 3-(4-(2-(2-Hydroxyethylamino)ethoxy)phenyl)-6,8-dimethylpyrimido[5,4-e][1,2,4]triazine-5,7(6H,8H)-dione; 3444342-Hydroxyethylamino)propoxy)phenyl)-6,8-dimethylpyrimido[5,4-e][1,2,4]triazine-5,7(6H,8H)-dione; 3-(4-(2-(2-(Dimethylamino)ethylamino)ethoxy)phenyl)-6,8-dimethylpyrimido[5,4-e][1,2,4]triazine-5,7(6H,8H)-dione; 3-(4-(3-(2-(Dimethylamino)ethylamino)propoxy)phenyl)-6,8-dimethylpyrimido[5,4-e][1,2,4]triazine-5,7(6H,8H)-dione; 3-(4-(2-(2-(Diethylamino)ethylamino)ethoxy)phenyl)-6,8-dimethylpyrimido[5,4-e][1,2,4]triazine-5,7(6H,8H)-dione; 3444342-(Diethylamino)ethylamino)propoxy)phenyl)-6,8-dimethylpyrimido[5,4-e][1,2,4]triazine-5,7(6H,8H)-dione; 6,8-Dimethyl-3-(4-(2-(piperidin-1-yl)ethoxy)phenyl)pyrimido[5,4-e][1,2,4]triazine-5,7(6H,8H)-dione; 6,8-Dimethyl-3-(4-(3-(piperidin-1-yl)propoxy)phenyl)pyrimido[5,4-e][1,2,4]triazine-5,7(6H,8H)-dione; 6,8-Dimethyl-3-(4-(3-morpholinopropoxy)phenyl)pyrimido[5,4-e][1,2,4]triazine-5,7(6H,8H)-dione; 6,8-Dimethyl-3-(4-(2-(piperazin-1-yl)ethoxy)phenyl)pyrimido[5,4-e][1,2,4]triazine-5,7(6H,8H)-dione; 6,8-Dimethyl-3-(4-(3-(4-methylpiperazin-1-yl)propoxy)phenyl)pyrimido[5,4-e][1,2,4]triazine-5,7(6H,8H)-dione; 3-(4-(2-(4-(2-Aminoacetyl)piperazin-1-yl)ethoxy)phenyl)-6,8-dimethylpyrimido[5,4-e][1,2,4]triazine-5,7(6H,8H)-dione; 3-(4-(3-(4-(2-Aminoacetyl)piperazin-1-ylpropoxy)phenyl)-6,8-dimethylpyrimido[5,4-e][1,2,4]triazine-5,7(6H,8H)-dione; 3-(4-(2-(3-Aminopyrrolidin-1-yl)ethoxy)phenyl)-6,8-dimethylpyrimido[5,4-e][1,2,4]triazine-5,7(6H,8H)-dione; 3-(4-(3-(3-Aminopyrrolidin-1-yl)propoxy)phenyl)-6,8-dimethylpyrimido[5,4-e][1,2,4]triazine-5,7(6H,8H)-dione; 3-(4-(2-Aminoethylamino)phenyl)-6,8-dimethylpyrimido[5,4-e][1,2,4]triazine-5,7(6H,8H)-dione; 3-(4-(3-Aminopropylamino)phenyl)-6,8-dimethylpyrimido[5,4-e][1,2,4]triazine-5,7(6H,8H)-dione; 3-(4-(2-(Dimethylamino)ethylamino)phenyl)-6,8-dimethylpyrimido[5,4-e][1,2,4]triazine-5,7(6H,8H)-dione; 3-(4-(3-(Dimethylamino)propylamino)phenyl)-6,8-dimethylpyrimido[5,4-e][1,2,4]triazine-5,7(6H,8H)-dione; 3-(4-(2-(Diethylamino)ethylamino)phenyl)-6,8-dimethylpyrimido[5,4-e][1,2,4]triazine-5,7(6H,8H)-dione; 3-(4-(3-(Diethylamino)propylamino)phenyl)-6,8-dimethylpyrimido[5,4-e][1,2,4]triazine-5,7(6H,8H)-dione; 3-(4-(2-(2-Hydroxyethylamino)ethylamino)phenyl)-6,8-dimethylpyrimido[5,4-e][1,2,4]triazine-5,7(6H,8H)-dione; 3444342-Hydroxyethylamino)propylamino)phenyl)-6,8-dimethylpyrimido[5,4-e][1,2,4]triazine-5,7(6H,8H)-dione; 6,8-Dimethyl-3-(4-(2-(piperidin-1-yl)ethylamino)phenyl)pyrimido[5,4-e][1,2,4]triazine-5,7(6H,8H)-dione; 6,8-Dimethyl-3-(4-(3-(piperidin-1-yl)propylamino)phenyl)pyrimido[5,4-e][1,2,4]triazine-5,7(6H,8H)-dione; 6,8-Dimethyl-3-(4-(2-morpholinoethylamino)phenyl)pyrimido[5,4-e][1,2,4]triazine-5,7(6H,8H)-dione; 6,8-Dimethyl-3-(4-(3-morpholinopropylamino)phenyl)pyrimido[5,4-e][1,2,4]triazine-5,7(6H,8H)-dione; 6,8-Dimethyl-3-(4-(2-(pyrrolidin-1-yl)ethylamino)phenyl)pyrimido[5,4-e][1,2,4]triazine-5,7(6H,8H)-dione; 6,8-Dimethyl-3-(4-(3-(pyrrolidin-1-yl)propylamino)phenyl)pyrimido[5,4-e][1,2,4]triazine-5,7(6H,8H)-dione; N-(2-Aminoethyl)-4-(6,8-dimethyl-5,7-dioxo-5,6,7,8-tetrahydropyrimido[5,4-e][1,2,4]triazin-3-yl)benzamide; N-(3-Aminopropyl)-4-(6,8-dimethyl-5,7-dioxo-5,6,7,8-tetrahydropyrimido[5,4-e][1,2,4]triazin-3-yl)benzamide; N-(3-(Diethylamino)propyl)-4-(6,8-dimethyl-5,7-dioxo-5,6,7,8-tetrahydropyrimido[5,4-e][1,2,4]triazin-3-yl)benzamide; 4-(6,8-Dimethyl-5,7-dioxo-5,6,7,8-tetrahydropyrimido[5,4-e][1,2,4]triazin-3-yl)-N-(2-(piperazin-1-yl)ethyl)benzamide;
4-(6,8-Dimethyl-5,7-dioxo-5,6,7,8-tetrahydropyrimido[5,4-e][1,2,4]triazin-3-yl)-N-(3-(piperazin-1-yl)propyl)benzamide; 3-(4-(2-Aminoethoxy)phenyl)-8-cyclopentyl-6-methylpyrimido[5,4-e][1,2,4]triazine-5,7(6H,8H)-dione; 3-(4-(3-Aminopropoxy)phenyl)-8-cyclopentyl-6-methylpyrimido[5,4-e][1,2,4]triazine-5,7(6H,8H)-dione; 8-Cyclopentyl-3-(4-(2-(dimethylamino)ethoxy)phenyl)-6-methylpyrimido[5,4-e][1,2,4]triazine-5,7(6H, 8H)-dione; 8-Cyclopentyl-3-(4-(3-(dimethylamino)propoxy)phenyl)-6-methylpyrimido[5,4-e][1,2,4]triazine-5,7(6H,8H)-dione; 8-Cyclopentyl-3-(4-(2-(diethylamino)ethoxy)phenyl)-6-methylpyrimido[5,4-e][1,2,4]triazine-5,7(6H,8H)-dione; 8-Cyclopentyl-3-(4-(3-(diethylamino)propoxy)phenyl)-6-methylpyrimido[5,4-e][1,2,4]triazine-5,7(6H,8H)-dione; 8-Cyclopentyl-3-(4-(2-(2-hydroxyethylamino)ethoxy)phenyl)-6-methylpyrimido[5,4-e][1,2,4]triazine-5,7(6H,8H)-dione; 8-Cyclopentyl-3-(4-(3-(2-hydroxyethylamino)propoxy)phenyl)-6-methylpyrimido[5,4-e][1,2,4]triazine-5,7(6H,8H)-dione; 8-Cyclopentyl-3-(4-(2-(2-(dimethylamino)ethylamino)ethoxy)phenyl)-6-methylpyrimido[5,4-e][1,2,4]triazine-5,7(6H,8H)-dione; 8-Cyclopentyl-3-(4-(3-(2-(dimethylamino)ethylamino)propoxy)phenyl)-6-methylpyrimido[5,4-e][1,2,4]triazine-5,7(6H,8H)-dione; 8-Cyclopentyl-3-(4-(2-(2-(diethylamino)ethylamino)ethoxy)phenyl)-6-methylpyrimido[5,4-e][1,2,4]triazine-5,7(6H,8H)-dione; 8-Cyclopentyl-3-(4-(3-(2-(diethylamino)ethylamino)propoxy)phenyl)-6-methylpyrimido[5,4-e][1,2,4]triazine-5,7(6H,8H)-dione; 8-Cyclopentyl-6-methyl-3-(4-(2-(piperidin-1-yl)ethoxy)phenyl)pyrimido[5,4-e][1,2,4]triazine-5,7(6H,8H)-dione; 8-Cyclopentyl-6-methyl-3-(4-(3-(piperidin-1-yl)propoxy)phenyl)pyrimido[5,4-e][1,2,4]triazine-5,7(6H,8H)-dione; 8-Cyclopentyl-6-methyl-3-(4-(2-morpholinoethoxy)phenyl))pyrimido[5,4-e][1,2,4]triazine-5,7(6H,8H)-dione; 8-Cyclopentyl-6-methyl-3-(4-(2-(piperazin-1-yl)ethoxy)phenyl)pyrimido[5,4-e][1,2,4]triazine-5,7(6H,8H)-dione; 8-Cyclopentyl-6-methyl-3-(4-(3-(piperazin-1-yl)propoxy)phenyl)pyrimido[5,4-e][1,2,4]triazine-5,7(6H,8H)-dione; 8-Cyclopentyl-6-methyl-3-(4-(2-(4-methylpiperazin-1-yl)ethoxy)phenyl)pyrimido[5,4-e][1,2,4]triazine-5,7(6H,8H)-dione; 8-Cyclopentyl-6-methyl-3-(4-(3-(4-methylpiperazin-1-yl)propoxy)phenyl)pyrimido[5,4-e][1,2,4]triazine-5,7(6H,8H)-dione; 3-(4-(2-(4-(2-Aminoacetyl)piperazin-1-yl)ethoxy)phenyl)-8-cyclopentyl-6-methylpyrimido[5,4-e][1,2,4]triazine-5,7(6H,8H)-dione; 3-(4-(3-(4-(2-Aminoacetyl)piperazin-1-yl)propoxy)phenyl)-8-cyclopentyl-6-methylpyrimido[5,4-e][1,2,4]triazine-5,7(6H,8H)-dione;
3-(4-(2-(3-Aminopyrrolidin-1-yl)ethoxy)phenyl)-8-cyclopentyl-6-methylpyrimido[5,4-e][1,2,4]triazine-5,7(6H,8H)-dione; 3-(4-(3-(3-Aminopyrrolidin-1-yl)propoxy)phenyl)-8-cyclopentyl-6-methylpyrimido[5,4-e][1,2,4]triazine-5,7(6H,8H)-dione; 3-(4-(2-Aminoethylamino)phenyl)-8-cyclopentyl-6-methylpyrimido[5,4-e][1,2,4]triazine-5,7(6H,8H)-dione; 3-(4-(3-Aminopropylamino)phenyl)-8-cyclopentyl-6-methylpyrimido[5,4-e][1,2,4]triazine-5,7(6H,8H)-dione; 8-Cyclopentyl-3-(4-(2-(dimethylamino)ethylamino)phenyl)-6-methylpyrimido[5,4-e][1,2,4]triazine-5,7(6H,8H)-dione; 8-Cyclopentyl-3-(4-(3-(dimethylamino)propylamino)phenyl)-6-methylpyrimido[5,4-e][1,2,4]triazine-5,7(6H,8H)-dione; 8-Cyclopentyl-3-(4-(2-(diethylamino)ethylamino)phenyl)-6-methylpyrimido[5,4-e][1,2,4]triazine-5,7(6H,8H)-dione;
8-Cyclopentyl-3-(4-(3-(diethylamino)propylamino)phenyl)-6-methylpyrimido[5,4-e][1,2,4]triazine-5,7(6H,8H)-dione; 8-Cyclopentyl-3-(4-(2-(2-hydroxyethylamino)ethylamino)phenyl)-6-methylpyrimido[5,4-e][1,2,4]triazine-5,7(6H,8H)-dione; 8-Cyclopentyl-3-(4-(3-(2-hydroxyethylamino)propylamino)phenyl)-6-methylpyrimido[5,4-e][1,2,4]triazine-5,7(6H,8H)-dione; 8-Cyclopentyl-6-methyl-3-(4-(2-(piperidin-1-yl)ethylamino)phenyl)pyrimido[5,4-e][1,2,4]triazine-5,7(6H,8H)-dione; 8-Cyclopentyl-6-methyl-3-(4-(3-(piperidin-1-yl)propylamino)phenyl)pyrimido[5,4-e][1,2,4]triazine-5,7(6H,8H)-dione; 8-Cyclopentyl-6-methyl-3-(4-(2-morpholinoethylamino)phenyl)pyrimido[5,4-e][1,2,4]triazine-5,7(6H,8H)-dione; 8-Cyclopentyl-6-methyl-3-(4-(3-morpholinopropylamino)phenyl)pyrimido[5,4-e][1,2,4]triazine-5,7(6H,8H)-dione; 8-Cyclopentyl-6-methyl-3-(4-(2-(pyrrolidin-1-yl)ethylamino)phenyl)pyrimido[5,4-e][1,2,4]triazine-5,7(6H,8H)-dione; 8-Cyclopentyl-6-methyl-3-(4-(3-(pyrrolidin-1-yl)propylamino)phenyl)pyrimido[5,4-e][1,2,4]triazine-5,7(6H,8H)-dione; N-(2-Aminoethyl)-4-(8-cyclopentyl-6-methyl-5,7-dioxo-5,6,7,8-tetrahydropyrimido[5,4-e][1,2,4]triazin-3-yl)benzamide; N-(3-Aminopropyl)-4-(8-cyclopentyl-6-methyl-5,7-dioxo-5,6,7,8-tetrahydropyrimido[5,4-e][1,2,4]triazin-3-yl)benzamide; 4-(8-Cyclopentyl-6-methyl-5,7-dioxo-5,6,7,8-tetrahydropyrimido[5,4-e][1,2,4]triazin-3-yl)-N-(2-(diethylamino)ethyl)benzamide; 4-(8-Cyclopentyl-6-methyl-5,7-dioxo-5,6,7,8-tetrahydropyrimido[5,4-e][1,2,4]triazin-3-yl)-N-(3-(diethylamino)propyl)benzamide; 4-(8-Cyclopentyl-6-methyl-5,7-dioxo-5,6,7,8-tetrahydropyrimido[5,4-e][1,2,4]triazin-3-yl)-N-(2-(piperazin-1-yl)ethyl)benzamide; 4-(8-Cyclopentyl-6-methyl-5,7-dioxo-5,6,7,8-tetrahydropyrimido[5,4-e][1,2,4]triazin-3-yl)-N-(3-(piperazin-1-yl)propyl)benzamide; 3-(4-(2-Aminoethoxy)phenyl)-8-(4-fluorophenyl)-6-methylpyrimido[5,4-e][1,2,4]triazine-5,7(6H,8H)-dione; 3-(4-(3-Aminopropoxy)phenyl)-8-(4-fluorophenyl)-6-methylpyrimido[5,4-e][1,2,4]triazine-5,7(6H,8H)-dione; 3-(4-(2-(Dimethylamino)ethoxy)phenyl)-8-(4-fluorophenyl)-6-methylpyrimido[5,4-e][1,2,4]triazine-5,7(6H,8H)-dione; 3-(4-(3-(Dimethylamino)propoxy)phenyl)-8-(4-fluorophenyl)-6-methylpyrimido[5,4-e][1,2,4]triazine-5,7(6H,8H)-dione; 3-(4-(2-(Diethylamino)ethoxy)phenyl)-8-(4-fluorophenyl)-6-methylpyrimido[5,4-e][1,2,4]triazine-5,7(6H,8H)-dione; 3-(4-(3-(Diethylamino)propoxy)phenyl)-8-(4-fluorophenyl)-6-methylpyrimido[5,4-e][1,2,4]triazine-5,7(6H,8H)-dione; 8-(4-Fluorophenyl)-3-(4-(2-(2-hydroxyethylamino)ethoxy)phenyl)-6-methylpyrimido[5,4-e][1,2,4]triazine-5,7(6H,8H)-dione; 8-(4-Fluorophenyl)-3-(4-(3-(2-hydroxyethylamino)propoxy)phenyl)-6-methylpyrimido[5,4-e][1,2,4]triazine-5,7(6H,8H)-dione; 3-(4-(2-(2-(Dimethylamino)ethylamino)ethoxy)phenyl)-8-(4-fluorophenyl)-6-methylpyrimido[5,4-e][1,2,4]triazine-5,7(6H,8H)-dione; 3444342-(Dimethylamino)ethylamino)propoxy)phenyl)-8-(4-fluorophenyl)-6-methylpyrimido[5,4-e][1,2,4]triazine-5,7(6H,8H)-dione; 3-(4-(2-(2-(Diethylamino)ethylamino)ethoxy)phenyl)-8-(4-fluorophenyl)-6-methylpyrimido[5,4-e][1,2,4]triazine-5,7(6H,8H)-dione; 3-(4-(3-(2-(Diethylamino)ethylamino)propoxy)phenyl)-8-(4-fluorophenyl)-6-methylpyrimido[5,4-e][1,2,4]triazine-5,7(6H,8H)-dione; 8-(4-Fluorophenyl)-6-methyl-3-(4-(2-(piperidin-1-yl)ethoxy)phenyl)pyrimido[5,4-e][1,2,4]triazine-5,7(6H,8H)-dione; 8-(4-Fluorophenyl)-6-methyl-3-(4-(3-(piperidin-1-yl)propoxy)phenyl)pyrimido[5,4-e][1,2,4]triazine-5,7(6H,8H)-dione; 8-(4-Fluorophenyl)-6-methyl-3-(4-(2-morpholinoethoxy)phenyl)pyrimido[5,4-e][1,2,4]triazine-5,7(6H,8H)-dione; 8-(4-Fluorophenyl)-6-methyl-3-(4-(3-morpholinopropoxy)phenyl)pyrimido[5,4-e][1,2,4]triazine-5,7(6H,8H)-dione; 8-(4-Fluorophenyl)-6-methyl-3-(4-(2-(piperazin-1-yl)ethoxy)phenyl)pyrimido[5,4-e][1,2,4]triazine-5,7(6H,8H)-dione; 8-(4-Fluorophenyl)-6-methyl-3-(4-(3-(piperazin-1-yl)propoxy)phenyl)pyrimido[5,4-e][1,2,4]triazine-5,7(6H,8H)-dione; 8-(4-Fluorophenyl)-6-methyl-3-(4-(2-(4-methylpiperazin-1-yl)ethoxy)phenyl)pyrimido[5,4-e][1,2,4]triazine-5,7(6H,8H)-dione; 8-(4-Fluorophenyl))-6-methyl-3-(4-(3-(4-methylpiperazin-1-yl)propoxy)phenyl)pyrimido[5,4-e][1,2,4]triazine-5,7(6H,8H)-dione; 3-(4-(2-(4-(2-Aminoacetyl)piperazin-1-yl)ethoxy)phenyl)-8-(4-fluorophenyl)-6-methylpyrimido[5,4-e][1,2,4]triazine-5,7(6H,8H)-dione; 3-(4-(3-(4-(2-Aminoacetyl)piperazin-1-yl)propoxy)phenyl)-8-(4-fluorophenyl)-6-methylpyrimido[5,4-e][1,2,4]triazine-5,7(6H,8H)-dione; 3-(4-(2-(3-Aminopyrrolidin-1-yl)ethoxy)phenyl)-8-(4-fluorophenyl)-6-methylpyrimido[5,4-e][1,2,4]triazine-5,7(6H,8H)-dione; 3-(4-(3-(3-Aminopyrrolidin-1-yl)propoxy)phenyl)-8-(4-fluorophenyl)-6-methylpyrimido[5,4-e][1,2,4]triazine-5,7(6H,8H)-dione; 3-(4-(2-Aminoethylamino)phenyl)-8-(4-fluorophenyl)-6-methylpyrimido[5,4-e][1,2,4]triazine-5,7(6H,8H)-dione; 3-(4-(3-Aminopropylamino)phenyl)-8-(4-fluorophenyl)-6-methylpyrimido[5,4-e][1,2,4]triazine-5,7(6H,8H)-dione; 3-(4-(2-(Dimethylamino)ethylamino)phenyl)-8-(4-fluorophenyl)-6-methylpyrimido[5,4-e][1,2,4]triazine-5,7(6H,8H)-dione; 3-(4-(3-(Dimethylamino)propylamino)phenyl)-8-(4-fluorophenyl)-6-methylpyrimido[5,4-e][1,2,4]triazine-5,7(6H,8H)-dione; 3-(4-(2-(Diethylamino)ethylamino)phenyl)-8-(4-fluorophenyl)-6-methylpyrimido[5,4-e][1,2,4]triazine-5,7(6H,8H)-dione; 3-(4-(3-(Diethylamino)propylamino)phenyl)-8-(4-fluorophenyl)-6-methylpyrimido[5,4-e][1,2,4]triazine-5,7(6H,8H)-dione; 8-(4-Fluorophenyl)-3-(4-(2-(2-hydroxyethylamino)ethyl(amino)phenyl)-6-methylpyrimido[5,4-e][1,2,4]triazine-5,7(6H,8H)-dione; 8-(4-Fluorophenyl)-3-(4-(3-(2-hydroxyethyl(amino)propylamino)phenyl)-6-methylpyrimido[5,4-e][1,2,4]triazine-5,7(6H,8H)-dione; 8-(4-Fluorophenyl)-6-methyl-3-(4-(2-(piperidin-1-yl)ethylamino)phenyl)pyrimido[5,4-d][1,2,4]triazine-5,7(6H,8H)-dione; 8-(4-Fluorophenyl)-6-methyl-3-(4-(3-(piperidin-1-yl)propylamino)phenyl)pyrimido[5,4-e][1,2,4]triazine-5,7(6H,8H)-dione; 8-(4-Fluorophenyl)-6-methyl-3-(4-(2-morpholinoethylamino)phenyl)pyrimido[5,4-e][1,2,4]triazine-5,7(6H,8H)-dione; 8-(4-Fluorophenyl)-6-methyl-3-(4-(3-morpholinopropylamino)phenyl)pyrimido[5,4-e][1,2,4]triazine-5,7(6H,8H)-dione; 8-(4-Fluorophenyl)-6-methyl-3-(4-(2-(pyrrolidin-1-yl)ethylamino)phenyl)pyrimido[5,4-e][1,2,4]triazine-5,7(6H,8H)-dione; 8-(4-Fluorophenyl)-6-methyl-3-(4-(3-(pyrrolidin-1-yl)propylamino)phenyl)pyrimido[5,4-e][1,2,4]triazine-5,7(6H,8H)-dione; N-(2-Aminoethyl)-4-(8-(4-fluorophenyl)-6-methyl-5,7-dioxo-5,6,7,8-tetrahydropyrimido[5,4-e]l[1,2,4]triazin-3-yl)benzamide; N-(3-Aminopropyl)-4-(8-(4-fluorophenyl))-6-methyl-5,7-dioxo-5,6,7,8-tetrahydropyrimido[5,4-e][1,2,4]triazin-3-yl)benzamide; N-(2-(Diethylamino)ethyl)-4-(8-(4-fluorophenyl)-6-methyl-5,7-dioxo-5,6,7,8-tetrahydropyrimido[5,4-e][1,2,4]triazin-3-yl)benzamide; N-(3-(Diethylamino)propyl)-4-(Q-(4-fluorophenyl)-6-methyl-5,7-dioxo-5,6,7,8-tetrahydropyrimido[5,4-e][1,2,4]triazin-3-yl)benzamide; 4-(8-(4-Fluorophenyl)-6-methyl-5,7-dioxo-5,6,7,8-tetrahydropyrimido[5,4-e][1,2,4]triazin-3-yl)-N-(2-(piperazin-1-yl)ethyl)benzamide; 4-(8-(4-Fluorophenyl)-6-methyl-5,7-dioxo-5,6,7,8-tetrahydropyrimido[5,4-e][1,2,4]triazin-3-yl)-N-(3-(piperazin-1-yl)propyl)benzamide; 3-(4-(2-Aminoethoxy)phenyl)-8-(3,4-difluorobenzyl)-6-methylpyrimido[5,4-e][1,2,4]triazine-5,7(6H,8H)-dione; 3-(4-(3-Aminopropoxy)phenyl)-8-(3,4-difluorobenzyl)-6-methylpyrimido[5,4-e][1,2,4]triazine-5,7(6H,8H)-dione; 8-(3,4-Difluorobenzyl)-3-(4-(2-(dimethylamino)ethoxy)phenyl)-6-methylpyrimido[5,4-e][1,2,4]triazine-5,7(6H,8H)-dione; 8-(3,4-Difluorobenzyl)-3-(4-(3-(dimethylamino)propoxy)phenyl)-6-methylpyrimido[5,4-e][1,2,4]triazine-5,7(6H,8H)-dione; 3-(4-(2-(Diethylamino)ethoxy)phenyl)-8-(3,4-difluorobenzyl)-6-methylpyrimido[5,4-e][1,2,4]triazine-5,7(6H,8H)-dione; 3-(4-(3-(Diethylamino)propoxy)phenyl)-8-(3,4-difluorobenzyl)-6-methylpyrimido[5,4-e][1,2,4]triazine-5,7(6H,8H)-dione; 8-(3,4-Difluorobenzyl)-3-(4-(2-(2-hydroxyethylamino)ethoxy)phenyl)-6-methylpyrimido[5,4-e][1,2,4]triazine-5,7(6H,8H)-dione; 8-(3,4-Difluorobenzyl)-3-(4-(3-(2-hydroxyethylamino)propoxy)phenyl)-6-methylpyrimido[5,4-e][1,2,4]triazine-5,7(6H,8H)-dione; 8-(3,4-Difluorobenzyl)-3-(4-(2-(2-(dimethylamino)ethylamino)ethoxy)phenyl)-6-methylpyrimido[5,4-e][1,2,4]triazine-5,7(6H,8H)-dione; 8-(3,4-Difluorobenzyl)-3-(4-(3-(2-(dimethylamino)ethylamino)propoxy)phenyl)-6-methylpyrimido[5,4-e][1,2,4]triazine-5,7(6H,8H)-dione; 3-(4-(2-(2-(Diethylamino)ethylamino)ethoxy)phenyl)-8-(3,4-difluorobenzyl)-6-methylpyrimido[5,4-e][1,2,4]triazine-5,7(6H,8H)-dione; 3444342-(Diethylamino)ethylamino)propoxy)phenyl)-8-(3,4-difluorobenzyl)-6-methylpyrimido[5,4-e][1,2,4]triazine-5,7(6H,8H)-dione; 8-(3,4-Difluorobenzyl)-6-methyl-3-(4-(2-(piperidin-1-yl)ethoxy)phenyl)pyrimido[5,4-e][1,2,4]triazine-5,7(6H,8H)-dione; 8-(3,4-Difluorobenzyl)-6-methyl-3-(4-(3-(piperidin-1-yl)propoxy)phenyl)pyrimido[5,4-e][1,2,4]triazine-5-dione; 8-(3,4-Difluorobenzyl)-6-methyl-3-(4-(2-(piperazin-1-yl)ethoxy)phenyl)pyrimido[5,4-e][1,2,4]triazine-5,7(6H,8H)-dione; 8-(3,4-Difluorobenzyl)-6-methyl-3-(4-(3-morpholinopropoxy)phenyl))pyrimido[5,4-e][1,2,4]triazine-5,7(6H,8H)-dione; Difluorobenzyl)-6-methyl-3-(4-(2-(4-methylpiperazin-1-yl)ethoxy)phenyl)pyrimido[5,4-e][1,2,4]triazine-5,7(6H,8H)-dione; 8-(3,4-Difluorobenzyl)-6-methyl-3-(4-(3-(piperazin-1-yl)propoxy)phenyl)pyrimido[5,4-e][1,2,4]triazine-5,7(6H,8H)-dione; 3-(4-(2-(4-(2-Aminoacetyl)piperazin-1-yl)ethoxy)phenyl)-8-(3,4-difluorobenzyl)-6-methylpyrimido[5,4-e][1,2,4]triazine-5,7(6H,8H)-dione; 3-(4-(3-(4-(2-Aminoacetyl)piperazin-1-yl)propoxy)phenyl)-8-(3,4-difluorobenzyl)-6-methylpyrimido[5,4-e][1,2,4]triazine-5,7(6H,8H)-dione; 3-(4-(2-(3-Aminopyrrolidin-1-yl)ethoxy)phenyl)-8-(3,4-difluorobenzyl)-6-methylpyrimido[5,4-e][1,2,4]triazine-5,7(6H,8H)-dione; 3-(4-(3-(3-Aminopyrrolidin-1-yl)propoxy)phenyl)-8-(3,4-difluorobenzyl)-6-methylpyrimido[5,4-e][1,2,4]triazine-5,7(6H,8H)-dione; 3-(4-(2-Aminoethylamino)phenyl)-8-(3,4-difluorobenzyl)-6-methylpyrimido[5,4-e][1,2,4]triazine-5,7(6H,8H)-dione; 3-(4-(3-Aminopropylamino)phenyl)-8-(3,4-difluorobenzyl)-6-methylpyrimido[5,4-e][1,2,4]triazine-5,7(6H,8H)-dione; 8-(3,4-Difluorobenzyl)-3-(4-(2-(dimethylamino)ethylamino)phenyl)-6-methylpyrimido[5,4-e][1,2,4]triazine-5,7(6H,8H)-dione; 8-(3,4-Difluorobenzyl)-3-(4-(3-(dimethylamino)propylamino)phenyl)-6-methylpyrimido[5,4-e][1,2,4]triazine-5,7(6H,8H)-dione; 3-(4-(2-(Diethylamino)ethylamino)phenyl)-8-(3,4-difluorobenzyl)-6-methylpyrimido[5,4-e][1,2,4]triazine-5,7(6H,8H)-dione; 3-(4-(3-(Diethylamino)propylamino)phenyl)-8-(3,4-difluorobenzyl)-6-methylpyrimido[5,4-e][1,2,4]triazine-5,7(6H,8H)-dione; 8-(3,4-Difluorobenzyl)-3-(4-(2-(2-hydroxyethylamino)ethylamino)phenyl)-6-methylpyrimido[5,4-e][1,2,4]triazine-5,7(6H,8H)-dione; 8-(3,4-Difluorobenzyl)-3-(4-(3-(2-hydroxyethylamino)propylamino)phenyl)-6-methylpyrimido[5,4-e][1,2,4]triazine-5,7(6H,8H)-dione; 8-(3,4-Difluorobenzyl)-6-methyl-3-(4-(2-(piperidin-1-yl)ethylamino)phenyl)pyrimido[5,4-e][1,2,4]triazine-5,7(6H,8H)-dione; 8-(3,4-Difluorobenzyl)-6-methyl-3-(4-(3-(piperidin-1-yl)propylamino)phenyl)pyrimido[5,4-e][1,2,4]triazine-5,7(6H,8H)-dione; 8-(3,4-Difluorobenzyl)-6-methyl-3-(4(2-morpholinoethylamino)phenyl)pyrimido[5,4-e][1,2,4]triazine-5,7(6H,8H)-dione; Difluorobenzyl)-6-methyl-3-(4-(3-morpholinopropylamino)phenyl)pyrimido[5,4-e][1,2,4]triazine-5,7(6H,8H)-dione; 8-(3,4-Difluorobenzyl)-6-methyl-3-(4-(2-(pyrrolidin-1-yl)ethylamino)phenyl)pyrimido[5,4-e][1,2,4]triazine-5,7(6H,8H)-dione; Difluorobenzyl)-6-methyl-3-(4-(3-(pyrrolidin-1-yl)propylamino)phenyl)pyrimido[5,4-e][1,2,4]triazine-5,7(6H,8H)-dione; N-(2-Aminoethyl)-4-(8-(3,4-difluorobenzyl-6-methyl-5,7-dioxo-5,6,7,8-tetrahydropyrimido[5,4-e][1,2,4]triazin-3-yl)benzamide; N-(3-Aminopropyl)-4-(8-(3,4-difluorobenzyl)-6-methyl-5,7-dioxo-5,6,7,8-tetrahydropyrimido[5,4-e][1,2,4]triazin-3-yl)benzamide; N-(2-(Diethylamino)ethyl)-4-(8-(3,4-difluorobenzyl)-6-methy)-5,7-dioxo-5,6,7,8-tetrahydropyrimido[5,4-d][1,2,4]triazin-3-yl)benzamide; N-(3-(Diethylamino)propyl)-4-(8-(3,4-difluorobenzyl)-6-methyl-5,7-dioxo-5,6,7,8-tetrahydropyrimido[5,4-e][1,2,4]triazin-3-yl)benzamide; 4-(8-(3,4-Difluorobenzyl)-6-methyl-5,7-dioxo-5,6,7,8-tetrahydropyrimido[5,4-e][1,2,4]triazin-3-yl)-N-(2-(piperazin-1-yl)ethyl)benzamide; 4-(8-(3,4-Difluorobenzyl)-6-methyl-5,7-dioxo-5,6,7,8-tetrahydropyrimido[5,4-e][1,2,4]triazin-3-yl)-N-(3-(piperazin-1-yl)propyl)benzamide; 3-(4-(2-Aminoethoxy)phenyl)-6-methyl-8-(pyridin-3-ylmethyl)pyrimido[5,4-e][1,2,4]triazine-5,7(6H,8H)-dione; 3-(4-(3-Aminopropoxy)phenyl)-6-methyl-8-(pyridin-3-ylmethyl)pyrimido[5,4-e][1,2,4]triazine-5,7(6H,8H)-dione; 3-(4-(2-(Dimethylamino)ethoxy)phenyl)-6-methyl-8-(pyridin-3-ylmethyl)pyrimido[5,4-e][1,2,4]triazine-5,7(61-1,8H)-dione; 3-(4-(3-(Dimethylamino)propoxy)phenyl)-6-methyl-8-(pyridin-3-ylmethyl)pyrimido[5,4-e][1,2,4]triazine-5,7(6H,8H)-dione; 3-(4-(2-(Diethylamino)ethoxy)phenyl)-6-methyl-8-(pyridin-3-ylmethyl)pyrimido[5,4-e][1,2,4]triazine-5,7(6H,8H)-dione; 3-(4-(3-(Diethylamino)propoxy)phenyl)-6-methyl-8-(pyridin-3-ylmethyl)pyrimido[5,4-e][1,2,4]triazine-5,7(6H,8H)-dione; 3-(4-(2-(2-Hydroxyethylamino)ethoxy)phenyl)-6-methyl-8-(pyridin-3-ylmethyl)pyrimido[5,4-e][1,2,4]triazine-5,7(6H,8H)-dione; 3-(4-(3-(2-Hydroxyethylamino)propoxy)phenyl)-6-methyl-8-(pyridin-3-ylmethyl)pyrimido[5,4-e][1,2,4]triazine-5,7(6H,8H)-dione; 3-(4-(2-(2-(Dimethylamino)ethylamino)ethoxy)phenyl)-6-methyl-8-(pyridin-3-ylmethyl)pyrimido[5,4-e][1,2,4]triazine-5,7(6H,8H)-dione; 3444342-(Dimethylamino)ethylamino)propoxy)phenyl)-6-methyl-8-(pyridin-3-ylmethyl)pyrimido[5,4-e][1,2,4]triazine-5,7(6H,8H)-dione; 3-(4-(2-(2-(Diethylamino)ethylamino)ethoxy)phenyl)-6-methyl-8-(pyridin-3-ylmethyl)pyrimido[5,4-e][1,2,4]triazine-5,7(6H,8H)-dione; 3-(4-(3-(2-(Diethylamino)ethylamino)propoxy)phenyl)-6-methyl-8-(pyridin-3-ylmethyl)pyrimido[5,4-e][1,2,4]triazine-5,7(6H,8H)-dione; 6-Methyl-3-(4-(2-(piperidin-1-yl)ethoxy)phenyl)-8-(pyridin-3-ylmethyl)pyrimido[5,4-e][1,2,4]triazine-5,7(6H,8H)-dione; 6-Methyl-3-(4-(3-(piperidin-1-yl)propoxy)phenyl)-8-(pyridin-3-ylmethyl)pyrimido[5,4-e][1,2,4]triazine-5,7(6H,8H)-dione; 6-Methyl-3-(4-(2-morpholinoethoxy)phenyl)-8-(pyridin-3-ylmethyl)pyrimido[5,4-e][1,2,4]triazine-5,7(6H,8H)-dione; 6-Methyl-3-(4-(3-morpholinopropoxy)phenyl)-8-(pyridin-3-ylmethyl)pyrimido[5,4-e][1,2,4]triazine-5,7(6H,8H)-dione; 6-Methyl-3-(4-(2-(piperazin-1-yl)ethoxy)phenyl)-8-(pyridin-3-ylmethyl)pyrimido[5,4-e][1,2,4]triazine-5,7(6H,8H)-dione; 6-Methyl-3-(4-(3-(piperazin-1-yl)propoxy)phenyl)-8-(pyridin-3-ylmethyl)pyrimido[5,4-e][1,2,4]triazine-5,7(6H,8H)-dione;
6-Methyl-3-(4-(2-(4-methylpiperazin-1-yl)ethoxy)phenyl)-8-(pyridin-3-ylmethyl)pyrimido[5,4-e][1,2,4]triazine-5,7(6H,8H)-dione; 6-Methyl-3-(4-(3-(4-methylpiperazin-1-yl)propoxy)phenyl)-8-(pyridin-3-ylmethyl)pyrimido[5,4-e][1,2,4]triazine-5,7(6H,8H)-dione; 3-(4-(2-(4-(2-Aminoacetyl)piperazin-1-yl)ethoxy)phenyl)-6-methyl-8-(pyridin-3-ylmethyl)pyrimido[5,4-e][1,2,4]triazine-5,7(6H,8H)-dione; 3-(4-(3-(4-(2-Aminoacetyl)piperazin-1-yl)propoxy)phenyl)-6-methyl-8-(pyridin-3-ylmethyl)pyrimido[5,4-e][1,2,4]triazine-5,7(6H,8H)-dione; 3-(4-(2-(3-Aminopyrrolidin-1-yl)ethoxy)phenyl)-6-methyl-8-(pyridin-3-ylmethyl)pyrimido[5,4-e][1,2,4]triazine-5,7(6H,8H)-dione; 3-(4(3-(3-Aminopyrrolidin-1-yl)propoxy)phenyl)-6-methyl-8-(pyridin-3-ylmethyl)pyrimido[5,4-e][1,2,4]triazine-5,7(6H,8H)-dione; 3-(4-(2-Aminoethylamino)phenyl)-6-methyl-8-(pyridin-3-ylmethyl)pyrimido[5,4-e][1,2,4]triazine-5,7(6H,8H)-dione; 3-(4-(3-Aminopropylamino)phenyl)-6-methyl-8-(pyridin-3-ylmethyl)pyrimido[5,4-e][1,2,4]triazine-5,7(6H,8H)-dione; 3-(4-(2-(Dimethylamino)ethylamino)phenyl)-6-methyl-8-(pyridin-3-ylmethyl)pyrimido[5,4-e][1,2,4]triazine-5,7(6H,8H)-dione; 3-(4-(3-(Dimethylamino)propylamino)phenyl)-6-methyl-8-(pyridin-3-ylmethyl)pyrimido[5,4e][1,2,4]triazine-5,7(6H,8H)-dione; 3-(4-(2-(Diethylamino)ethylamino)phenyl)-6-methyl-8-(pyridin-3-ylmethyl)pyrimido[5,4-e][1,2,4]triazine-5,7(6H,8H)-dione; 3-(4-(3-(Diethylamino)propylamino)phenyl)-6-methyl-8-(pyridin-3-ylmethyl)pyrimido[5,4-e][1,2,4]triazine-5,7(6H,8H)-dione; 3-(4-(2-(2-Hydroxyethylamino)ethylamino)phenyl)-6-methyl-8-(pyridin-3-ylmethyl)pyrimido[5,4-e][1,2,4]triazine-5,7(6H,8H)-dione; 3-(4-(3-(2-Hydroxyethylamino)propylamino)phenyl)-6-methyl-8-(pyridin-3-ylmethyl)pyrimido[5,4-e][1,2,4]triazine-5,7(6H,8H)-dione; 6-Methyl-3-(4-(2-(piperidin-1-yl)ethylamino)phenyl)-8-(pyridin-3-ylmethyl)pyrimido[5,4-e][1,2,4]triazine-5,7(6H,8H)-dione; 6-Methyl-3-(4-(3-(piperidin-1-yl)propylamino)phenyl)-8-(pyridin-3-ylmethyl)pyrimido[5,4-e][1,2,4]triazine-5,7(6H,8H)-dione; 6-Methyl-3-(4-(2-morpholinoethylamino)phenyl)-8-(pyridin-3-ylmethyl)pyrimido[5,4-e][1,2,4]triazine-5,7(6H,8H)-dione; 6-Methyl-3-(4-(3-morpholinopropylamino)phenyl)-8-(pyridin-3-ylmethyl)pyrimido[5,4-e][1,2,4]triazine-5,7(6H,8H)-dione; 6-Methyl-8-(pyridin-3-ylmethyl)-3-(4-(2-(pyrrolidin-1-yl)ethylamino)phenyl)pyrimido[5,4-e][1,2,4]triazine-5,7(6H,8H)-dione; 6-Methyl-8-(pyridin-3-ylmethyl)-3-(4-(3-(pyrrolidin-1-yl)propylamino)phenyl)pyrimido[5,4-e][1,2,4]triazine-5,7(6H,8H)-dione; N-(2-Aminoethyl)-4-(6-methyl-5,7-dioxo-8-(pyridin-3-ylmethyl)-5,6,7,8-tetrahydropyrimido[5,4-e][1,2,4]triazin-3-yl)benzamide; N-(3-Aminopropyl)-4-(6-methyl-5,7-dioxo-8-(pyridin-3-ylmethyl)-5,6,7,0-tetrahydropyrimido[5,4-e][1,2,4]triazin-3-yl)benzamide; N-(2-(Diethylamino)ethyl)-4-(6-methyl-5,7-dioxo-8-(pyridin-3-ylmethyl)-5,6,7,8-tetrahydropyrimido[5,4-e][1,2,4]triazin-3-yl)benzamide; N-(3-(Diethylamino)propyl)-4-(6-methyl-5,7-dioxo-8-(pyridin-3-ylmethyl)-5,6,7,8-tetrahydropyrimido[5,4-e][1,2,4]triazin-3-yl)benzamide; 4-(6-Methyl-5,7-dioxo-8-(pyridin-3-ylmethyl)-5,6,7,8-tetrahydropyrimido[5,4-e][1,2,4]triazin-3-yl)-N-(2-(piperazin-1-yl)ethyl)benzamide; 4-(6-Methyl-5,7-dioxo-8-(pyridin-3-ylmethyl)-5,6,7,8-tetrahydropyrimido[5,4-e][1,2,4]triazin-3-yl)-N-(3-(piperazin-1-yl)propyl)benzamide;
1-Benzyl-6-(4-(2-(diethylamino)ethoxy)phenyl)-3-methylpyrimido[5,4-d]pyrimidine-2,4(1H,3H)-dione; 1-(4-Fluorobenzyl)-3-methyl-6-(4-(2-(4-methylpiperazin-1-yl)ethoxy)phenyl)pyrimido[5,4-d]pyrimidine-2,4(1H,3H)-dione; 1-(3,4-Difluorobenzyl)-3-methyl-6-(4-(2-(morpholin-4-yl)ethoxy)phenyl)pyrimido[5,4-d]pyrimidine-2,4(1H,3H)-dione; 6-(4-(2-(4-Aminoacetylpiperazin-1-yl)ethoxy)phenyl)-3-methyl-1-phenylpyrimido[5,4-d]pyrimidine-2,4(1H,3H)-dione; 6-(4-(2-(4-(Dimethylamino)piperidin-1-yl)ethoxy)phenyl)-1-isopropyl-3-methylpyrimido[5,4-d]pyrimidine-2,4(1H,3H)-dione; 6-(4-(2-(3-Aminopyrrolidin-1-yl)ethoxy)phenyl)-1-(3-fluorophenyl)-3-methylpyrimido[5,4-d]pyrimidine-2,4(1H,3H)-dione; 1-Cyclopentyl-3-methyl-6-(4-(3-(morpholin-4-yl)propyloxy)phenyl)pyrimido[5,4-d]pyrimidine-2,4(1H,3H)-dione; 1-Cyclopropyl-6-(6-(2-(diethylamino)ethoxy)pyridyl-3-yl)-3-methylpyrimido[5,4-d]pyrimidine-2,4(1H,3H)-dione; 1-Cyclopentyl-6-(4-(2-(piperidin-1-yl)ethoxy)phenyl)pyrimido[5,4-d]pyrimidine-2,4(1H,3H)-dione; 6-(4-(2-(Dimethylamino)ethyl)phenyl)-3-methyl-1-phenylpyrimido[5,4-d]pyrimidine-2,4(1H,3H)-dione; 1-(3,4-Difluorobenzyl)-3-methyl-6-(4-(3-(4-methylpiperazin-1-yl)propyl)phenyl)pyrimido[5,4-d]pyrimidine-2,4(1H,3H)-dione; 1-Isopropyl-3-methyl-6-(4-(2-(piperidin-1-yl)ethylamino)phenyl)pyrimido[5,4-d]pyrimidine-2,4(1H,3H)-dione; 1-Cyclopropyl-3-(2-(piperidin-1-yl)ethyl)-6-(pyridyl-3-yl)pyrimido[5,4-d]pyrimidine-2,4(1H,3H)-dione; 3-(4-(2-Aminoethyl)benzyl)-1-cyclopentyl-6-(4-fluorophenyl)pyrimido[5,4-d]pyrimidine-2,4(1H,3H)-dione; 1-Cyclopentyl-6-(4-carboxyphenyl)-3-methylpyrimido[5,4-d]pyrimidine-2,4(1H,3H)-dione; 6-(4-(2-(Diethylamino)ethylcarboxamide)phenyl)-1,3-dimethylpyrimido[5,4-d]pyrimidine-2,4(1H,3H)-dione; 1,3-Dimethyl-6-(4-(2-(4-methylpiperazin-1-yl)ethoxy)phenyl)pyrimido[5,4-d]pyrimidine-2,4(1H,3H)-dione; 1-Methyl-6-(4-(2-(4-methylpiperazin-1-yl)ethoxy)phenyl)pyrimido[5,4-d]pyrimidine-2,4(1H,3H)-dione; 6-(4-(2-(Diethylamino)ethoxy)phenyl)-1,3-dimethylpyrimido[5,4-d]pyrimidine-2,4(1H,3H)-dione; 6-(4-(2-(Diethylamino)ethoxy)phenyl)-1-methylpyrimido[5,4-d]pyrimidine-2,4(1H,3H)-dione; 1,3-Dimethyl-6-(4-(2-(morpholin-4-yl)ethoxy)phenyl)pyrimido[5,4-d]pyrimidine-2,4(1H,3H)-dione; 6-(4-(2-(4-Methylpiperazin-1-yl)ethoxy)phenyl)-1,3,8-trimethylpyrimido[5,4-d]pyrimidine-2,4(1H,3H)-dione; 1,8-Dimethyl-6-(4-(2-(4-methylpiperazin-1-yl)ethoxy)phenyl)pyrimido[5,4-d]pyrimidine-2,4(1H,3H)-dione; 6-(4-(2-(Diethylamino)ethoxy)phenyl)-1,3,8-trimethylpyrimido[5,4-d]pyrimidine-2,4(1H,3H)-dione; 6-(4-(2-(Diethylamino)ethoxy)phenyl)-1,8-dimethylpyrimido[5,4-d]pyrimidine-2,4(1H,3H)-dione; 6-(4-(2-(Morpholin-4-yl)ethoxy)phenyl)-1,3,8-trimethylpyrimido[5,4-d]pyrimidine-2,4(1H,3H)-dione; and mixtures thereof, and pharmaceutically acceptable salts and hydrates thereof.

Compounds having a formula I, II, or III can be prepared according to the syntheses outlined in Schemes 1-7.

Scheme 1 is a method for the preparation of compounds having a formula I. Compounds having a formula (5′) or a formula (8), for example, can be prepared via the synthetic route shown in Scheme I.

The synthesis of compounds of formula uses 3-substituted 6-chlorouracil (1) (e.g., 6-chloro-3-methyluracil) as a starting material. Nucleophilic attack of an appropriately substituted hydrazine having a formula NH₂NHR¹ (e.g., methylhydrazine or 2-(hydroxyethyl)hydrazine) onto compound (I) furnishes hydrazinylpyrimidine-2,4(1H,3H)-dione (2). Next, an aldehyde having a formula R²C(O)H is coupled with the hydrazinylpyrimidine-2,4(1H,3H)-dione to obtain hydrazone (3). Nitrosation and ring closure is accomplished by treatment with sodium nitrite in acetic acid-water, affording a mixture of pyrimidotriazinedione (5) and the corresponding N-oxide derivative (4). Treatment of the mixture with dithiothreitol provides the N¹-substituted pyrimidotriazinedione I. When R¹ and R³ are both methyl, the synthesis furnishes the N¹-methylpyrimidotriazinedione (5′). Additionally, when R¹ is methyl and R³ is 2-hydroxyethyl, the synthesis furnishes the N¹-(2-hydroxyethyl)pyrimidotriazinedione (8).

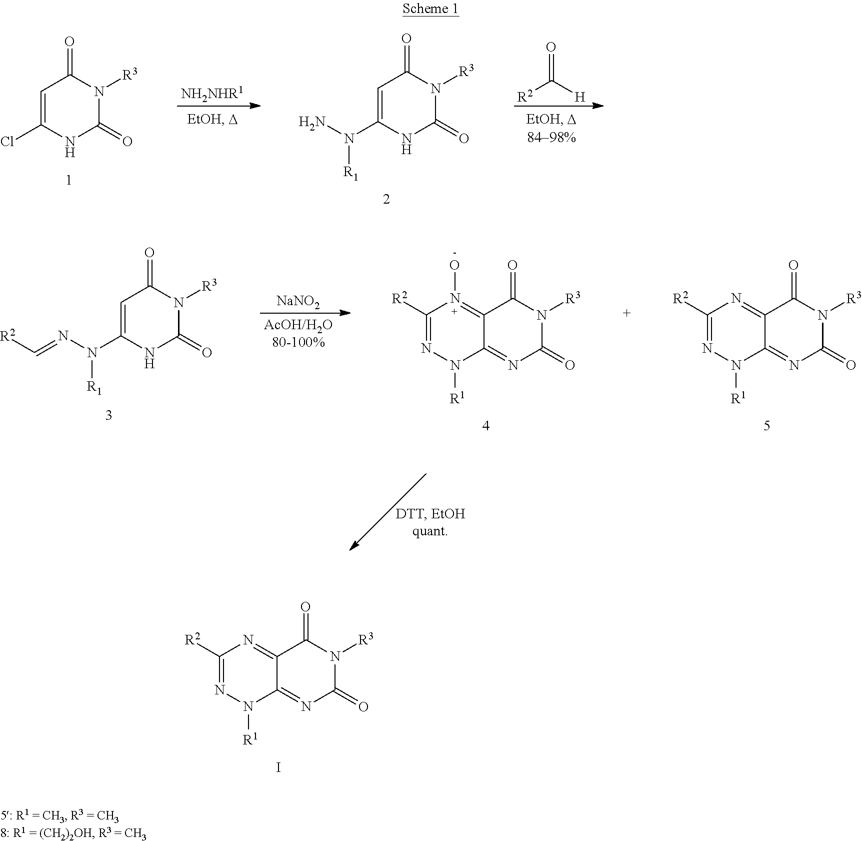

Scheme 2 is a method for the preparation of compounds having a formula II. Compounds having a formula (7), for example, can be prepared via the synthetic route shown in Scheme 2.

The synthesis of compounds of formula II follows the same initial steps as the synthesis of compounds of formula I, using 3-substituted 6-chlorouracil (1) as a starting material. Nucleophilic attack of methylhydrazine onto compound (I) furnishes hydrazinylpyrimidine-2,4(1H,3H)-dione (2). Next, an aldehyde having a formula R⁵C(O)H is coupled with the hydrazinylpyrimidine-2,4(1H,3H)-dione to obtain hydrazone (3). Nitrosation and ring closure is accomplished by treatment with sodium nitrite in acetic acid-water affording a mixture of pyrimidotriazinedione (5) and the corresponding N-oxide derivative (4).

After formation of the mixture of compounds (4) and (5), the synthetic route diverges from the reaction steps used to prepare the compounds of formula I. Reaction of the mixture of compounds (4) and (5) in DMF at 90° C. affords N¹-demethylated pyrimidotriazinedione (6). Alkylation of the N¹-demethylated pyrimidotriazinedione (6) with an alkyl halide in acetone using cesium carbonate as the base yields the N⁸-substituted pyrimidotriazinedione II. When R⁶ is methyl, the synthesis furnishes the N⁶-methylpyrimidotriazinedione (7).

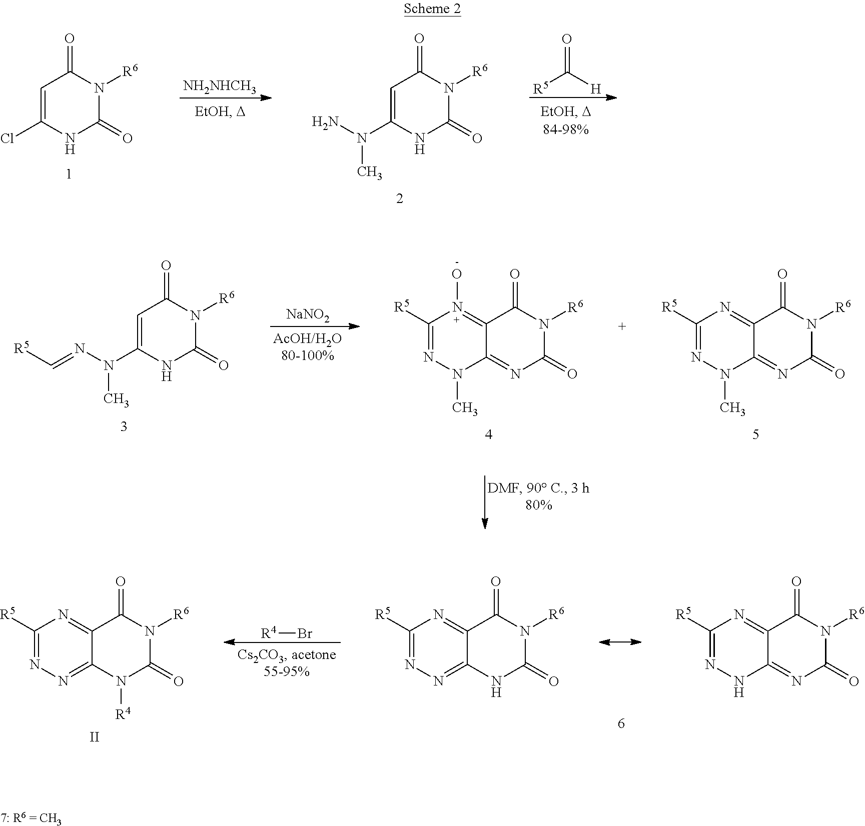

Scheme 3 is a method for the preparation of a subset of compounds having a formula II, wherein R⁵ is selected from the group consisting of phenol and alkylated phenol. Compounds having a formula (10), for example, can be prepared via the synthetic route shown in Scheme 3.

Compound (10) is synthesized from a compound of formula (7′). The compound of formula (7′) can be obtained according to Scheme 2 by using ortho-, meta-, or para-anisaldehyde as the condensing aldehyde to form hydrazone (3). Thus, the methoxy phenyl group of compound (7′) is demethylated with BBr₃ in CH₂Cl₂, furnishing compound (9). Phenol (9) then is alkylated by 2-(diethylamino)ethyl chloride hydrochloride with cesium carbonate in acetone to yield pyrimidotriazinedione (10). Compound (9) also can be alkylated with any suitable alkyl halide, for example, 2-(4-methylpiperazin-1-yl)ethyl chloride, 2-(pyrrolidin-1-yl)ethyl chloride, and 2-(N,N-diethylamino)ethyl chloride, to furnish alkylated phenols of formula II.

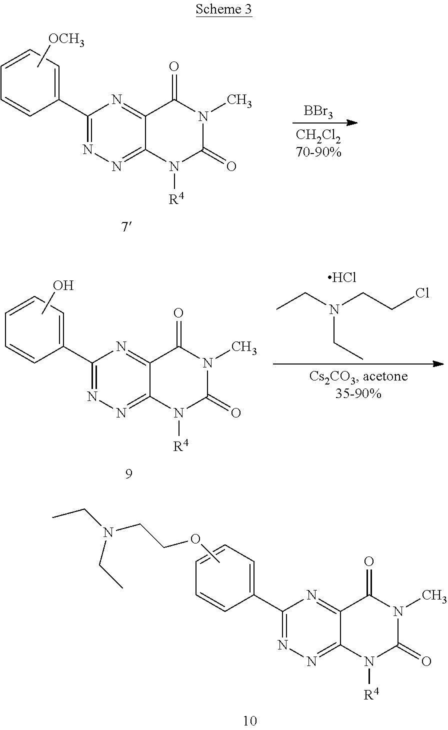

Scheme 4 is a method for the preparation of compounds having a formula I. Compounds having a formula (17), for example, can be prepared via the synthetic route shown in Scheme 4.

Compound (17) is synthesized via the route shown in Scheme 4, using the commercially available 6-chlorouracil (11) as starting material. The N¹ of 6-chlorouracil is selectively protected with a benzyloxymethyl (BOM) group, forming compound (12). Alkylation at N⁴ (N⁶ in the final pyrimidotriazinediones) is accomplished using an alkyl halide in the presence of cesium carbonate, yielding N-substituted chlorouracil (13). Treatment of compound (13) with methylhydrazine furnishes N¹-BOM-protected 6-(1-methylhydrazinyl)uracil (14). Next, an aldehyde having a formula R²C(O)H is coupled with compound (14) to obtain hydrazone (15). Nitrosation, ring closure, and in situ deprotection of the BOM group is accomplished by treatment with sodium nitrite in acetic acid-water affording a mixture of pyrimidotriazinedione (17) and the corresponding N-oxide derivative (16). Treatment of the mixture with dithiothreitol provides N¹-methylpyrimidotriazinedione (17).

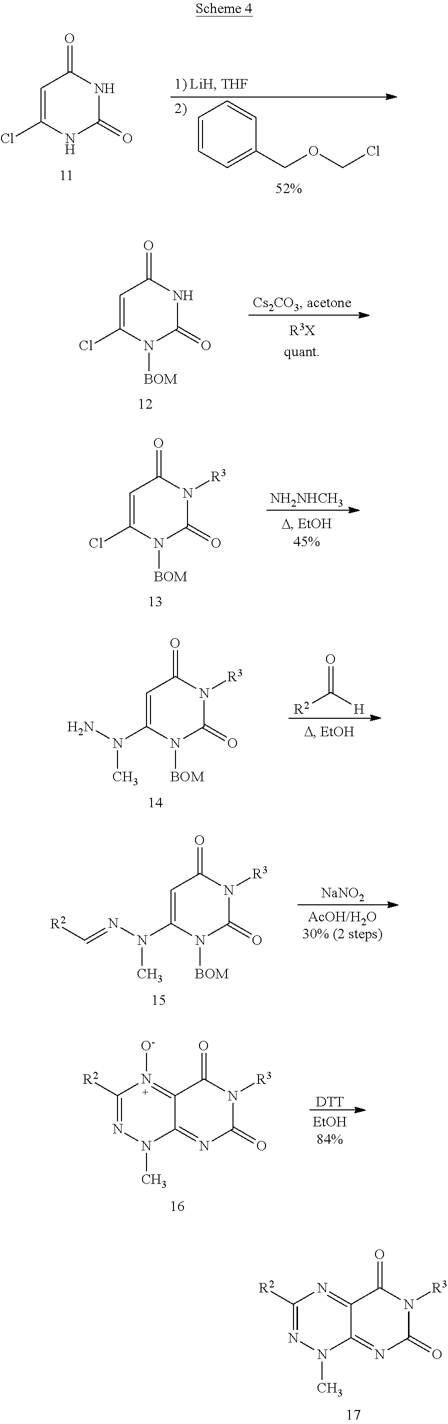

The method for preparing the compounds of formula II shown in Scheme 2 relies on a nucleophilic SN2 substitution reaction with an alkyl halide to introduce the R⁴ substituent. When alkyl halides were used, the reaction frequently required heating and/or long incubation times. An alternative method for the preparation of compounds having a formula II that does not require SN2 chemistry is shown in Scheme 5. Compounds having a formula (26), for example, can be prepared via the synthetic route shown in Scheme 5.

Compound (26) is synthesized via the route shown in Scheme 5. Diethyl malonate (18) and a monosubstituted urea (19) are coupled to form cyclic compound (20). An exemplary monosubstituted urea is N-phenylurea. A mixture of chlorides (2)) and (22) then is formed upon treatment of compound (20) with POCl₃. Nucleophilic attack of methylhydrazine onto chloride (22) furnishes hydrazinylpyrimidine-2,4(1H,3H)-dione (23). Next, an aldehyde having a formula R⁵C(O)H is coupled with the hydrazinylpyrimidine-2,4(1H,3H)-dione to obtain hydrazone (24). Nitrosation and ring closure is accomplished by treatment with sodium nitrite in acetic acid-water, affording a mixture of pyrimidotriazinedione (26) and the corresponding N-oxide derivative (25). Treatment of the mixture with dithiothreitol provides the N⁸-phenyl substituted pyrimidotriazinedione (26).

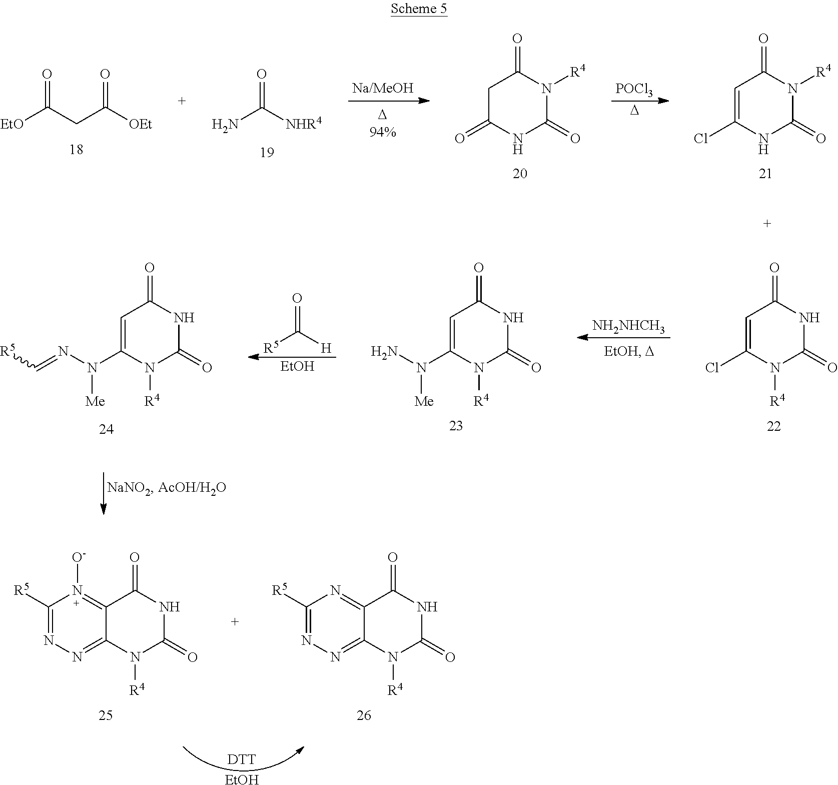

Compounds of formula I having various R¹ substituents can be prepared as shown in Scheme 6. Compounds having a formula (32), for example, can be prepared via the synthetic route shown in Scheme 6.

Compound (32) is synthesized via the route shown in Scheme 6. Nitration of 6-chloro-3-methyluracil (1) is accomplished using a mixture of nitric acid and sulfuric acid to furnish compound (27). In parallel, an aldehyde having a formula R²C(O)H is coupled with a monosubstituted hydrazine (29) to obtain hydrazone (30). An exemplary monosubstituted hydrazine is phenylhydrazine. Coupling of compound (27) and compound (30) furnishes hydrazone (31), which then can be cyclized using a mixture of zinc and ammonium chloride in ethanol-water to form N¹-substituted pyrimidotriazinedione (32).

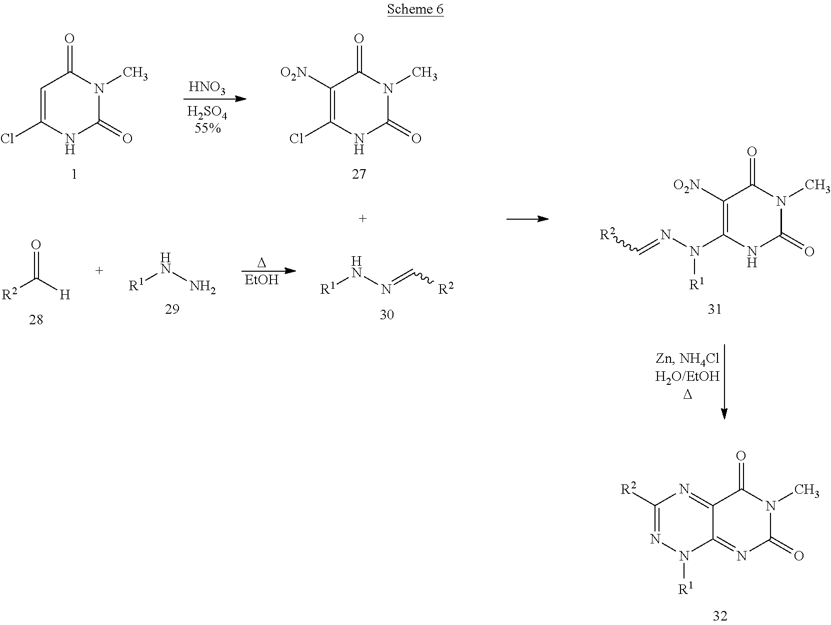

Scheme 7 is a method for the preparation of compounds having a formula III. Reaction of nitrouracil (33) with a primary amine having a formula NH₂CH₂R⁸ affords pyrazolo[4,3-d]pyrimidine 1-oxide (34). Treatment of compound (34) with sodium ethoxide in ethanol at reflux furnishes the substituted pyrimido[4,5-d]pyrimidine III.

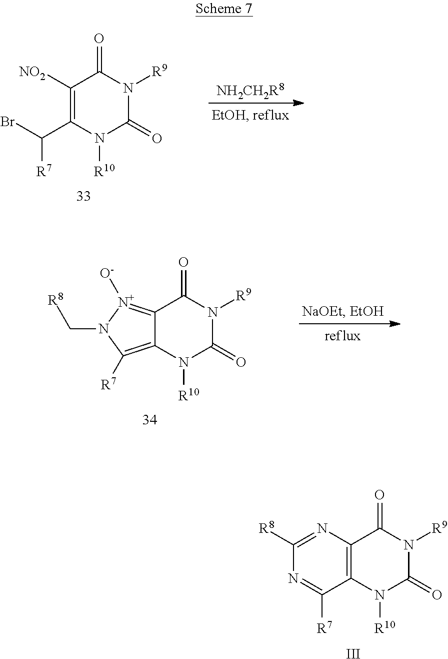

Scheme 8 is a method for the preparation of a subset of compounds having a formula II, wherein R⁵ is selected from the group consisting of optionally substituted alkylamino (e.g., compound (40)) and optionally substituted alkoxy (e.g., compound (41)).

Compounds (40) and (41) are synthesized from a compound of formula (35). Intermediate compound (39) can be obtained according to the methods of Senga et al. (J. Org. Chem. 43:469-472 (1978)). Briefly, nucleophilic attack of hydrazine onto compound (35) furnishes hydrazinylpyrimidine-2,4(1H,3H)-dione (36). Nitrosation is accomplished by treatment with sodium nitrite in acetic acid-water, affording compound (37). Next, formic acid is added to obtain the N-oxide compound (38). Treatment with POCl₃ provides 3-chloro pyrimidotriazinedione (39).

Compound (40) is obtained by reacting compound (39) with an appropriately substituted amine having a formula NHR¹¹R¹² in the presence of triethylamine. Exemplary amines include amines wherein R¹¹ is hydrogen or optionally substituted C₁₋₂₀ alkyl (e.g., C₁₋₂₀ aminoalkyl, C₁₋₂₀ hydroxyalkyl, or C₁₋₂₀ alkoxy(C₁₋₂₀ alkyl)) and R¹² is hydrogen or C₁₋₂₀ alkyl, or R¹¹ and R¹² taken together with the nitrogen atom to which they are bound form an optionally substituted 3 to 10 membered heterocyclic ring.

Compound (41) is obtained by reacting compound (39) with an appropriately substituted alcohol having a formula HOR¹³ in the presence of sodium hydride. Exemplary alcohols include alcohols wherein R¹³ is optionally substituted C₁₋₂₀ alkyl (e.g., C₁₋₂₀ aminoalkyl, C₁₋₂₀ hydroxyalkyl, or C₁₋₂₀ alkoxy(C₁₋₂₀ alkyl)).

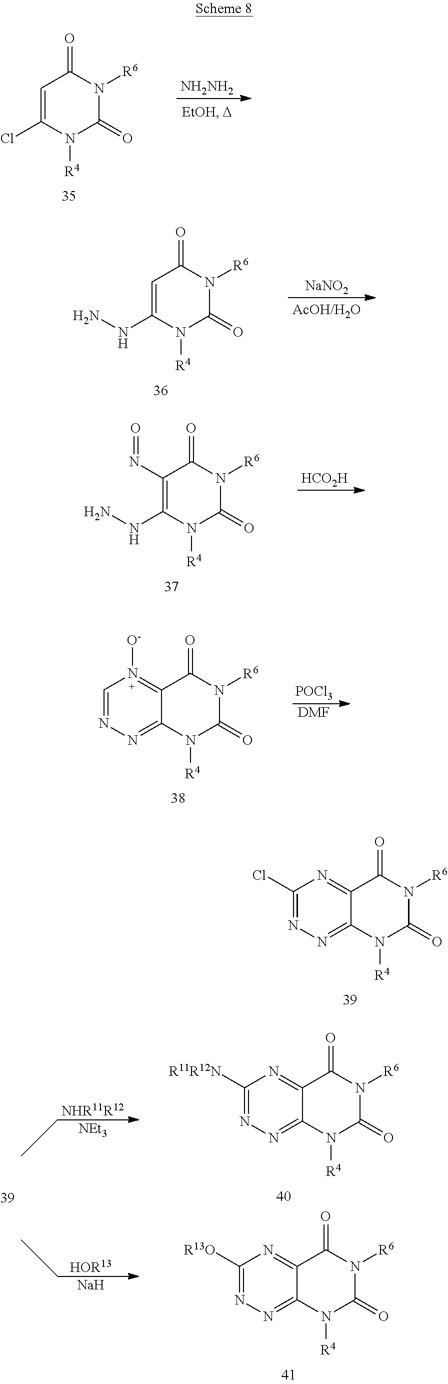

Scheme 9 is a method for the preparation of a subset of compounds having a formula II, wherein R⁵ is selected from the group consisting of optionally substituted aminomethyl (e.g., compound (44)), optionally substituted acylaminomethyl (e.g., compound (45)), and optionally substituted carboxamido (e.g., compound (47)).

Compounds (44), (45), and (47) are synthesized from a compound of formula (26′), which can be obtained according to Scheme 5. Intermediate compounds (43) and (46) can be obtained according to the methods of Pfleiderer et al. (J. Het. Chem. 33:949-960 (1996)). Briefly, reaction of compound (26′) with an appropriately substituted bromide having a formula R⁶Br in the presence of cesium carbonate affords compound (42). Bromination of compound (42) with NBS affords compound (43).

Compound (44) is obtained by treating compound (43) with an appropriately substituted amine having a formula NHR¹¹R¹² in the presence of triethylamine. Exemplary amines include amines wherein R¹¹ is hydrogen or optionally substituted C₁₋₂₀ alkyl (e.g., C₁₋₂₀ aminoalkyl, C₁₋₂₀ hydroxyalkyl, or C₁₋₂₀ alkoxy(C₁₋₂₀ alkyl)) and R¹² is hydrogen or C₁₋₂₀ alkyl, or R¹¹ and R¹² taken together with the nitrogen atom to which they are bound form an optionally substituted 3 to 10 membered heterocyclic ring. When R¹² is hydrogen, compound (45) can be obtained by amidation of compound (44) with an appropriately substituted carboxylic acid having a formula R¹⁴CO₄₁ in the presence of an activating reagent such as EDC (1-ethyl-3-(3-d)methylaminopropyl)carbodiimide). Exemplary carboxylic acids include carboxylic acids wherein R¹⁴ is optionally substituted C₁₋₂₀ alkyl (e.g., C₁₋₂₀ aminoalkyl, C₁₋₂₀ hydroxyalkyl, C₁₋₂₀ alkoxy(C₁₋₂₀ alkyl), C₁₋₂₀ alkyl(C₁₋₂₀ aminoalkyl), or C₁₋₂₀ acyl(C₁₋₂₀ aminoalkyl)). Exemplary carboxylic acids also include amino acids and protected variants and derivatives thereof. Suitable amino acid protecting groups include, but are not limited to t-butyloxycarbonyl, 9H-fluoren-9-ylmethoxycarbonyl, benzyloxy-carbonyl, and allyloxycarbonyl. Protecting groups can be removed according to known procedures to yield compounds of formula II.

Alternatively, compound'(42) is treated with potassium permanganate to form compound (46). Compound (47) is then obtained by treating compound (46) with an activating reagent such as EDC (1-ethyl-3-(3-dimethylaminopropyl)carbodiimide) and an appropriately substituted amine having a formula NHR¹¹R¹². Exemplary amines include amines wherein R¹¹ is hydrogen or optionally substituted C₁₋₂₀ alkyl (e.g., C₁₋₂₀ aminoalkyl, C₁₋₂₀ hydroxyalkyl, or C₁₋₂₀ alkoxy(C₁₋₂₀ alkyl)) and R¹² is hydrogen or C₁₋₂₀ alkyl, or R¹¹ and R¹² taken together with the nitrogen atom to which they are bound form an optionally substituted 3 to 10 membered heterocyclic ring.

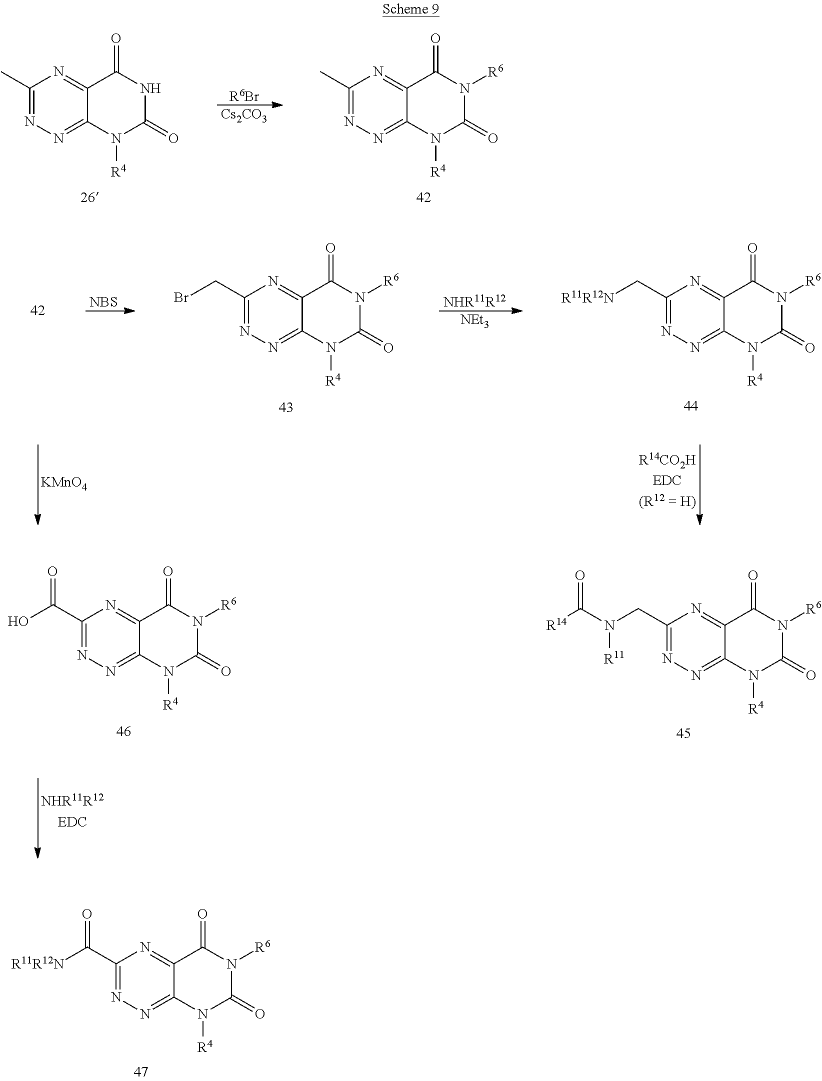

Scheme 10 is a method for the preparation of a subset of compounds having a formula II, wherein R⁵ is selected from the group consisting of aniline and alkylated aniline. Compounds having a formula (50), for example, can be prepared via the synthetic route shown in Scheme 10.

Compound (50) is synthesized from a compound of formula (48). The compound of formula (48) can be obtained according to Scheme 2 by using ortho-, meta-, or para-nitrobenzaldehyde as the condensing aldehyde to form hydrazone (3). Reduction of nitrophenyl compound (48) with iron metal in acetic acid furnishes aniline (49). Aniline (49) then is alkylated by an appropriately substituted alkyl halide (e.g., R¹⁵R¹⁶N(CH₂)ₙCl) with cesium carbonate in acetone to yield pyrimidotriazinedione (50). Suitable alkyl halides include alkyl halides having a formula R¹⁵R¹⁶N(CH₂)ₙCl wherein n is 1 to 20, preferably 2 to 3, and R¹⁵ and R¹⁶ are each independently selected from the group consisting of hydrogen and optionally substituted C₁₋₂₀ alkyl (e.g., C₁₋₂₀ aminoalkyl, C₁₋₂₀ hydroxyalkyl, or C₁₋₂₀ alkoxy(C₁₋₂₀ alkyl)), or R¹⁵ and R¹⁶ taken together with the nitrogen atom to which they are bound form an optionally substituted 3 to 10 membered heterocyclic ring. Compound (49) also can be alkylated with any suitable alkyl halide to furnish alkylated anilines of formula II.

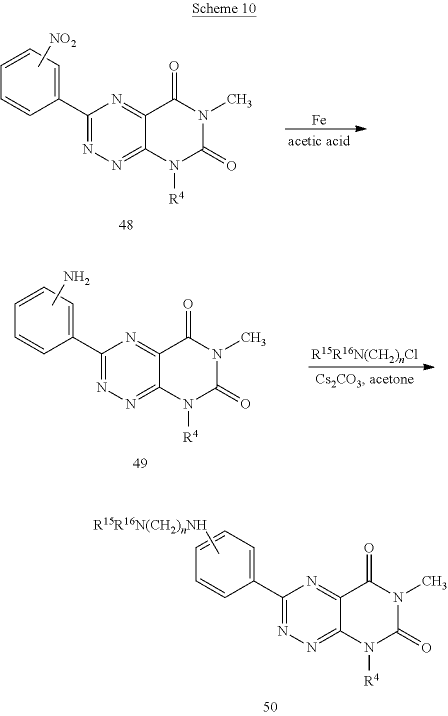

### EXAMPLES

The following examples are provided for illustration and are not intended to limit the scope of the invention. It should be appreciated by those of skill in the art that many changes can be made in the specific embodiments which are disclosed and still obtain a like or similar result without departing from the spirit and scope of the invention.

Melting points were determined in open capillary tubes on a Laboratory Devices MEL-TEMP apparatus and are uncorrected. The NMR spectra were recorded on a Bruker 500 MHz spectrometer with either CDCl₃, d-DMSO, or d-TFA as solvent. Chemical shift values are recorded in 6 units (ppm). Mass spectra (MS) were recorded on a Micromass TOFSPEC-2E Matrix-Assisted, Laser-Desorption, Time-of-Flight Mass Spectrometer.

### Example 1

### Synthesis of Hydrazinylpyrimidine-2,4(1H,3H)-diones (2a) and (2b)

(2a) 3-Methyl-6-(1-methylhydrazinyl)pyrimidine-2,4(1H,3H)-dione was prepared according to the methods of Daves, G. et al. (Journal of the American Chemical Society 84:1724-1729 (1962)).

(2b) 6-(1-(2-Hydroxyethyl)hydrazinyl)-3-methylpyrimidine-2,4(1H,3H)-dione. To a suspension of 6-chloro-3-methyluracil (3.0 g, 0.019 mol) in 25 mL absolute ethanol was added 2-(hydroxyethyl)hydrazine (6.3 mL, 0.093 mol, 5 eq). The mixture was heated at reflux for 3 h when TLC (20% MeOH/CH₂Cl₂) indicated consumption of the starting uracil. The reaction then was cooled to room temperature and concentrated. The resulting residue was triturated with ethyl acetate to yield a pale tan solid, which was collected by filtration to yield (2b) (1.33 g, 35%); mp 169-170° C.; ¹H NMR (d-DMSO) δ 3.04 (s, 3H), 3.33 (s, 2H), 3.43 (t, 5.5 Hz, 2H), 3.62 (t, 5.5 Hz, 2H), 4.73 (s, 1H).

### Example 2

### Synthesis of Hydrazones (3)

The following compounds were synthesized according to the method of Nagamatsu, et al. (Chemical & Pharmaceutical Bulletin 41:362-368 (1993)).

1. 6-(2-(4-Methoxybenzylidene)-1-methylhydrazinyl)-3-methylpyrimidine-2,4(1H,3H)-dione (R₁=CH₃, R₃=p-CH₃O—C₆H₄). 57 mg (66% yield), mp 231-233° C., ¹H NMR (CDCl₃) δ 3.34 (s, 6H), 3.89 (s, 3H), 5.12 (s, 1H), 6.98 (d, J=8.65 Hz, 2H), 7.63 (d, J=6.2 Hz, 2H), 7.68 (s, 1H), 9.20 (s, 1H).

2. 6-(2-(2-Fluorobenzylidene)-1-methylhydrazinyl)-3-methylpyrimidine-2,4(1H,3H)-dione (R₁=CH₃, R₃=o-F—C₆H₄). 143 mg (88% yield), mp 263-264° C., ¹H NMR (CDCl₃) δ 3.28 (s, 3H), 3.37 (s, 3H), 5.17 (s, 1H), 7.15 (m, 2H), 7.41 (m, 1H), 7.71 (m, 1H), 7.94 (s, 1H), 9.13 (s, 1H).

3. 3-Methyl-6-(1-methyl-2-(4-nitrobenzylidene)hydrazinyl)pyrimidine-2,4(1H,3H)-dione (R₁=CH₃, R₃=p-NO₂—C₆H₄). 156 mg (84% yield), mp 296-299° C., ¹H NMR (d-DMSO) δ 3.05 (s, 3H), 3.42 (s, 3H), 5.33 (s, 1H), 8.07 (s, 1H), 8.26 (s, 4H), 10.99 (s, 1H).

4. 3-Methyl-6-(1-methyl-2-(3-nitrobenzylidene)hydrazinyl)pyrimidine-2,4(1H,3H)-dione (R₁=CH₃, R₃=m-NO₂—C₆H₄). 161 mg (87% yield), mp 305-308° C., ¹H NMR (d-DMSO) δ 3.13 (s, 3H), 5.30 (s, 1H), 7.71 (m, 1H), 8.12 (s, 1H), 8.20 (t, J=6.0 Hz, 1H), 8.47 (t, J=6.7 Hz, 1H), 8.82 (s, 1H), 11.08 (s, 1H).

5. 3-Methyl-6-(1-methyl-2-(pyridin-2-ylmethylene)hydrazinyl)pyrimidine-2,4(1H,3H)-dione (R₁=CH₃, R₃=o-C₅H₄N). 120 mg (86% yield), mp 261-265° C., ¹H NMR (d-DMSO) δ 3.13 (s, 3H), 3.40 (s, 3H), 5.34 (s, 1H), 7.38 (m, 1H), 7.86 (m, 2H), 8.52 (d, J=8.0 Hz, 1H), 8.60 (d, J=4.25 Hz, 1H), 10.84 (s, 1H).

6. 6-(2-(4-(Dimethylamino)benzylidene)-1-methylhydrazinyl)-3-methylpyrimidine-2,4(1H,3H)-dione (R₁=CH₃, R₃=p-N(CH₃)₂—C₆H₄). 383 mg (% yield), mp 240-242° C., ¹H NMR (d-DMSO) δ 2.97 (s, 6H), 3.10 (s, 3H), 3.30 (s, 3H), 5.15 (s, 1H), 6.72 (d, J=8.7 Hz, 2HH), 7.77 (d, J=8.4 Hz, 2H), 7.86 (s, 1H), 10.44 (s, 1H).

7. 6-(2-(4-Fluorobenzylidene)-1-methylhydrazinyl)-3-methylpyrimidine-2,4(1H,3H)-dione (R₁=CH₃, R₃=p-F—C₆H₄). 478 mg (92% yield), mp 269-272° C., ¹H NMR (CDCl₃) δ 3.34 (s, 6H), 5.15 (s, 1H), 7.15 (d, J=8.6 Hz, 2H), 7.69 (d, J=8.6 Hz, 2H), 7.70 (s, 1H), 9.16 (s, 1H).

8. 6-(2-(3-Chlorobenzylidene)-1-methylhydrazinyl)-3-methylpyrimidine-2,4(1H,3H)-dione (R₁=CH₃, R₃=m—Cl—C₆H₄). 288 mg (84% yield), NMR (CDCl₃) δ 3.33 (s, 3H), 3.41 (s, 3H), 5.17 (s, 1H), 7.39 (d, J=6.2 Hz, 2H), 7.56 (d, J=6.35 Hz, 1H), 7.65 (s, 1H), 7.68 (s, 1H), 9.20 (s, 1H).

9. 6-(2-(4-(2-(Diethylamino)ethoxy)benzylidene)-1-methylhydrazinyl)-3-methylpyrimidine-2,4(1H,3H)-dione (R₁=CH₃, R₃=p-[(CH₃CH₂)₂NCH₂CH₂O]—C₆H₄). 134 mg (31% yield), mp 175-176° C., ¹H NMR (d-DMSO) δ 0.97 (t, J=7.0, 6H), 2.56 (q, J=7.1 Hz, 4H), 2.79 (t, J=7.0 Hz, 2H), 3.11 (s, 3H), 3.32 (s, 3H), 4.06 (t, J=7.0 Hz, 2H), 5.20 (s, 1H), 6.98 (d, J=8.4 Hz, 2H), 7.90 (d, J=8.2 Hz, 2H).

10. 6-(2-Ethylidene-1-methylhydrazinyl)-3-methylpyrimidine-2,4(1H,3H)-dione (R₁=CH₃, R₃=CH₃). 120 mg (57% yield), ¹H NMR (CDCl₃) δ 1.17 (t, J=7.5 Hz, 3H), 2.43 (m, 2H), 3.16 (s, 3H), 3.32 (s, 3H), 5.01 (s, 1H), 7.07 (s, 1H), 9.17 (s, 1H).

11. 6-(1-(2-Hydroxyethyl)-2-(4-methoxybenzylidene)hydrazinyl)-3-methylpyrimidine-2,4(1H,3H)-dione (R₁=CH₂CH₂OH, R₃=p-CH₃O—C₆H₄). 145 mg (91% yield), mp 234-235° C., ¹H NMR (d-DMSO) δ 3.11 (s, 3H), 3.62 (t, J=5.6 Hz, 2H), 3.80 (s, 3H), 3.99 (t, J=5.6 Hz, 2H), 5.08 (br s, 1H), 5.21 (s, 1H), 7.00 (d, J=8.3 Hz, 2H), 7.95 (d, J=8.2 Hz, 2H), 8.08 (s, 1H), 10.45 (br s, 1H).

12. 6-(1-(2-Hydroxyethyl)-2-(4-methylbenzylidene)hydrazinyl)-3-methylpyrimidine-2,4(1H,3H)-dione (R₁=CH₂CH₂OH, R₃=p-CH₃—C₆H₄). 140 mg (93% yield), mp 255-257° C., ¹H NMR (d-DMSO) δ 2.37 (s, 3H), 3.11 (s, 3H), 3.62 (t, J=5.5 Hz, 2H), 4.00 (t, J=5.5 Hz, 2H), 5.04 (br s, 1H), 5.25 (s, 1H), 7.25 (d, J=7.6 Hz, 2H), 7.85 (d, J=7.6 Hz, 2H), 8.09 (s, 1H), 10.46 (br s, 1H).

13. 6-(2-(4-Chlorobenzylidene)-1-(2-hydroxyethyl)hydrazinyl)-3-methylpyrimidine-2,4(1H,3H)-dione (R₁=CH₂CH₂OH, R₃=p—Cl—C₆H₄). 150 mg (93% yield), mp 258-260° C., ¹H NMR (d-DMSO) 83.12 (s, 3H), 3.63 (t, J=5.3 Hz, 2H), 4.02 (t, J=5.3 Hz, 2H), 5.03 (br s, 1H), 5.27 (s, 1H), 7.49 (d, J=8.4 Hz, 2H), 8.03 (d, J=8.4 Hz, 2H), 8.11 (s, 1H), 10.65 (br s, 1H).

14. 6-(2-(4-(Dimethylamino)benzylidene)-1-(2-hydroxyethyl)hydrazinyl)-3-methylpyrimidine-2,4(1H,3H)-dione (R₁=CH₂CH₂OH, R₃=p-(CH₃)₂N—C₆H₄). 156 mg (94% yield), mp 258-260° C., NMR (d-DMSO) δ 2.90 (s, 6H), 3.11 (s, 3H), 3.55 (t, J=5.2 Hz, 2H), 4.01 (t, J=5.2 Hz, 2H), 5.26 (s, 1H), 6.75 (d, J=8.2 Hz, 2H), 7.70 (d, J=8.2 Hz, 2H), 8.01 (s, 1H).

15. 6-(1-(2-Hydroxyethyl)-2-(pyridin-2-ylmethylene)hydrazinyl)-3-methylpyrimidine-2,4(1H,3H)-dione (R₁=CH₂CH₂OH, R₃=o-C₅H₄N). 131 mg (91% yield), mp 256-258° C., ¹H NMR (d-DMSO) δ 3.11 (s, 3H), 3.65 (t, J=5.3 Hz, 2H), 4.08 (t, J=5.3 Hz, 2H), 5.08 (br s, 1H), 5.33 (s, 1H), 7.38 (t, J=5.8 Hz, 1H), 7.86 (t, J=7.5 Hz, 1H), 8.07 (s, 1H), 8.51 (d, J=7.5 Hz, 1H), 8.59 (s, 1H), 10.70 (br s, 1H).

16. 6-(2-(3,4-Dichlorobenzylidene)-1-(2-hydroxyethyl)hydrazinyl)-3-methylpyrimidine-2,4(1H,3H)-dione (R₁=CH₂CH₂OH, R₃=3,4-di-Cl—C₆H₃). 178 mg (100% yield), mp 252-255° C., ¹H NMR (d-DMSO) δ 3.16 (s, 3H), 3.66 (t, J=5.2 Hz, 2H), 4.10 (t, J=5.2 Hz, 2H), 4.91 (br s, 1H), 5.22 (s, 1H), 7.44 (d, J=8.8 Hz, 1H), 7.65 (d, J=7.4 Hz, 1H), 7.80 (s, 1H), 8.12 (s, 1H), 10.29 (br s, 1H).

17. 6-(2-(3,4-Dimethoxybenzylidene)-1-(2-hydroxyethyl)hydrazinyl)-3-methylpyrimidine-2,4(1H,3H)-dione (R₁=CH₂CH₂OH, R₃=3,4-di-CH₃O—C₆H₃). 301 mg (86% yield), mp 226-228° C., ¹H NMR (d-DMSO) δ 3.12 (s, 3H), 3.63 (t, J=5.4 Hz, 2H), 3.80 (s, 3H), 3.85 (s, 3H), 3.99 (t, J=5.4 Hz, 2H), 4.91 (br s, 1H), 5.22 (s, 1H), 7.02 (d, J=8.4 Hz, 1H), 7.43 (d, J=7.9 Hz, 1H), 7.57 (s, 1H), 8.06 (s, 1H), 10.29 (br s, 1H).

18. 6-(2-(4-(2-(Diethylamino)ethoxy)benzylidene)-1-(2-hydroxyethyl)hydrazinyl)-3-methylpyrimidine-2,4(1H,3H)-dione (R₁=CH₂CH₂OH, R₃=p-[(CH₃CH₂)₂NCH₂CH₂O]—C₆H₄). 38 mg (12% yield), ¹H NMR (d-DMSO) δ 0.99 (t, J=7.0 Hz, 6H), 2.58 (q, J=6.9 Hz, 4H), 2.80 (t, J=5.5 Hz, 2H), 3.11 (s, 3H), 3.62 (t, J=5.5 Hz, 2H), 4.00 (t, J=5.5 Hz, 2H), 4.07 (t, J=5.5 Hz, 2H), 5.22 (s, 1H), 6.99 (d, J=8.3 Hz, 2H), 7.90 (d, J=8.2 Hz, 2H), 8.07 (s, 1H).

19. 6-(1-(2-Hydroxyethyl)-2-(3-nitrobenzylidene)hydrazinyl)-3-methylpyrimidine-2,4(1H,3H)-dione (R₁=CH₁CH₂OH, R₃=m-NO₂—C₆H₄). 165 mg (79% yield), ¹H NMR (d-DMSO) δ 3.13 (s, 3H), 3.66 (t, J=5.5 Hz, 2H), 4.05 (t, J=5.5 Hz, 2H), 5.04 (br s, 1H), 5.33 (s, 1H), 7.72 (t, J=7.8 Hz, 1H), 8.21 (d, J=7.9 Hz, 1H), 8.26 (s, 1H), 8.49 (d, J=7.8 Hz, 1H), 8.79 (s, 1H), 10.91 (s, 1H).

### Example 3

### Synthesis of Pyrimido[5,4-e][1,2,4]triazine-5,7(1H,6H)-diones (5) and (8)

### General Procedure A

An appropriately substituted hydrazone (3) (0.16 mmol) in 1 mL glacial acetic acid and 0.06 mL H₂O was stirred under nitrogen and cooled to 0° C. Sodium nitrite (0.25 mmol, 1.5 eq) was added, and the reaction mixture was allowed to warm to room temperature while stirring. Stirring was continued until reaction of the starting material was complete, thereby furnishing a mixture of the pyrimidotriazinedione and the corresponding N-oxide derivative. Diethyl ether was added to facilitate further precipitation, and the precipitate was collected by filtration. The precipitate then was suspended in 2 mL absolute ethanol under nitrogen, and dithiothreitol (0.5 mmol, 3.1 eq) was added. The mixture was stirred for 24-72 h until mass spectrometry analysis and/or TLC indicated complete conversion of the N-oxide to the reduced pyrimidotriazinedione. The precipitate was collected by filtration and recrystallized from ethanol if necessary.

The following compounds were synthesized according to General Procedure A.

(5b) 1,3,6-Trimethylpyrimido[5,4-e][1,2,4]triazine-5,7(1H,6H)-dione. 30 mg

(37% yield), mp 160-170° C. (dec), ¹H NMR (CDCl₃/TFA) δ 2.96 (s, 3H), 3.63 (s, 3H), 4.49 (s, 3H).

(5c) 3-Ethyl-1,6-dimethylpyrimido[5,4-e][1,2,4]triazine-5,7(1H,6H)-dione. 29 mg (62% yield), mp 174-175° C. (dec), ¹H NMR (CDCl₃/TFA) δ 1.45 (t, J=7.5 Hz, 3H), 3.22 (q, J=7.5 Hz, 2H), 3.58 (s, 3H), 4.46 (s, 3H).

(5d) 1,6-Dimethyl-3-phenylpyrimido[5,4-e][1,2,4]triazine-5,7(1H,6H)-dione. 20 mg (52% yield), mp 216-218° C. (dec), ¹H NMR (d-DMSO) δ 3.30 (s, 3H), 4.07 (s, 3H), 7.60 (m, 3H), 8.21 (m, 3H), MS 270.1 (M+H).

(5e) 1,6-Dimethyl-3-(4-methylphenyl)pyrimido[5,4-e][1,2,4]triazine-5,7(1H,6H)-dione. 20 mg (39% yield), mp 223-225° C., ¹H NMR (d-TFA) δ 2.42 (s, 3H), 3.35 (s, 3H), 3.41 (s, 3H), 7.41 (d, J=8.9 Hz, 2H), 7.59 (d, J=8.9 Hz, 2H), MS 284.1 (M+H).

(5f) 3-(4-Fluorophenyl)-1,6-dimethylpyrimido[5,4-e][1,2,4]triazine-5,7(1H,6H)-dione. 35 mg (66% yield), mp 213-216° C., ¹H NMR (d-DMSO) δ 3.29 (s, 3H), 4.05 (s, 3H), 7.44 (d, J=8.6 Hz, 2H), 8.24 (d, J=8.6 Hz, 2H).

(5g) 3-(2-Fluorophenyl)-1,6-dimethylpyrimido[5,4-e][1,2,4]triazine-5,7(1H,6H)-dione. 15 mg (28% yield), mp 191-194° C., ¹H NMR (d-DMSO) δ 3.28 (s, 3H), 4.04 (s, 3H), 7.44 (m, 2H), 7.65 (m, 1H), 7.98 (d, J=7.0 Hz, 1H), MS 288.4 (M+H).

(5h) 3-(4-Chlorophenyl)-1,6-dimethylpyrimido[5,4-e][1,2,4]triazine-5,7(1H,6H)-dione. 12 mg (47% yield), mp 218-223° C. (dec), ¹H NMR (d-DMSO) δ 3.33 (s, 3H), 3.92 (s, 3H), 7.52 (d, J=7.2 Hz, 2H), 7.75 (d, J=7.2 Hz, 2H), MS 304.0, 306.0 (M+H).

(5i) 3-(3-Chlorophenyl)-1,6-dimethylpyrimido[5,4-e][1,2,4]triazine-5,7(1H,6H)-dione. 41 mg (80% yield), mp 214-217° C., ¹H NMR (CDCl₃/TFA) δ 3.61 (s, 3H), 4.43 (s, 3H), 7.53 (t, J=7.9 Hz, 1H), 7.64 (d, J=7.55 Hz, 1H), 8.25 (d, J=7.6 Hz, 1H), 8.24 (d, J=7.6 Hz, 1H), 8.31 (s, 1H).

(5j) 3-(3,4-Dichlorophenyl)-1,6-dimethylpyrimido[5,4-e][1,2,4]triazine-5,7(1H,6H)-dione was prepared according to the procedure of Yoneda, F. et al.

(5k) 3-(4-Methoxyphenyl)-1,6-dimethylpyrimido[5,4-e][1,2,4]triazine-5,7(1H,6H)-dione 0.30 mg (58% yield), mp 245-250° C. (dec), NMR (d-TFA) δ 3.60 (s, 3H), 3.94 (s, 3H), 4.60 (s, 3H), 7.10 (d, J=9.05 Hz, 2H), 8.34 (d, J=9.05 Hz, 2H), MS 300.1 (M+H).

### (5l) 3-(4-(2-(Diethylamino)ethoxy)phenyl)-1,6-dimethylpyrimido[5,4-e][1,2,4]triazine-5,7(1H,6H)-dione. 15 mg (87% yield), mp 255-260° C. (dec), NMR (d-DMSO) δ 1.25 (t, J=7.0 Hz, 6H), 3.25 (q, J=7.0 Hz, 4H), 3.29 (s, 3H), 3.56 (t, J=7.1 Hz, 2H), 4.05 (s, 3H), 4.41 (t, J=7.1 Hz, 2H), 7.21 (d, J=8.3 Hz, 2H), 8.18 (d, J=7.9 Hz, 214).

(5m) 1,6-Dimethyl-3-(5-methylfuran-2-yl)pyrimido[5,4-e][1,2,4]triazine-5,7(1H,6H)-dione. 241 mg (73% yield), ¹H NMR (CDCl₃) δ 2.40 (s, 3H), 3.52 (s, 3H), 4.20 (s, 3H), 6.25 (s, 1H), 7.37 (s, 1H).

(5n) 3-(4-(Dimethylamino)phenyl)-1,6-dimethylpyrimido[5,4-e][1,2,4]triazine-5,7(1H,6H)-dione. 25 mg (73% yield), ¹H NMR (d-TFA) δ 3.37 (s, 6H), 3.57 (s, 3H), 4.59 (s, 3H), 7.78 (d, J=8.1 Hz, 2H), 8.59 (d, J=8.1 Hz, 2H).

(5o) 1,6-Dimethyl-3-(4-nitrophenyl)pyrimido[5,4-e][1,2,4]triazine-5,7(1H,6H)-dione. 20 mg (39% yield), mp 220-223° C., ¹H NMR (d-TFA) δ 3.62 (s, 3H). 4.63 (s, 3H), 8.43 (d, J=7.7 Hz, 2H), 8.61 (d, J=7.7 Hz, 2H), MS 315.1 (M+H).

(5p) 1,6-Dimethyl-3-(3-nitrophenyl)pyrimido[5,4-e][1,2,4]triazine-5,7(1H,6H)-dione. 21 mg (40% yield), mp 223-224° C., ¹H NMR (d-TFA) δ 3.63 (s, 3H), 4.66 (s, 3H), 7.84 (m, 1H), 8.54 (d, J=8.0 Hz, 1H), 8.82 (d, J=8.0 Hz, 1H), 9.25 (s, 114), MS 315.1 (M+H).

(5q) 1,6-Dimethyl-3-(pyridin-2-yl)pyrimido[5,4-e][1,2,4]triazine-5,7(1H,6H)-dione. 24 mg (24% yield), mp 298° C. (dec), ¹H NMR (d-DMSO) δ 2.51 (s, 3H), 3.27 (s, 3H), 7.51 (d, J=4.85 Hz, 1H), 7.99 (t, J=7.20 Hz, 1H), 8.38 (d, J=7.85 Hz, 1H), 8.75 (s, 1H).

(8a) 1-(2-Hydroxyethyl)-6-methyl-3-phenylpyrimido[5,4-e][1,2,4]triazine-5,7(1H,6H)-dione. 22 mg (27% yield), mp 200-201° C., ¹H NMR (d-DMSO) δ 3.22 (s, 3H), 3.95 (t, J=5.7 Hz, 2H), 4.55 (t, J=5.7 Hz, 2H), 7.61 (m, 3H), 8.21 (m, 214), MS 300.1 (M+H).

(8b) 1-(2-Hydroxyethyl)-6-methyl-3-(4-methylphenyl)pyrimido[5,4-e][1,2,4]triazine-5,7(1H,6H)-dione. 21 mg (41% yield), mp 201-203° C., ¹H NMR (d-TFA) δ 2.41 (s, 3H), 3.61 (s, 3H), 4.53 (t, J=5.2 Hz, 2H), 5.10 (t, J=5.3 Hz, 2H), 7.36 (d, J=8.0 Hz, 2H), 8.21 (d, J=8.1 Hz, 2H), MS 314.1 (M+H).

(8c) 3-(4-Chlorophenyl)-1-(2-hydroxyethyl)-6-methylpyrimido[5,4-e][1,2,4]triazine-5,7(1H,6H)-dione. 11 mg (30% yield), mp 201-205° C. (dec), ¹H NMR (d-DMSO) δ 3.30 (s, 3H), 3.95 (t, J=5.5 Hz, 2H), 4.55 (t, J=5.5 Hz, 2H), 4.99 (1, 1H), 7.69 (d, J=7.6 Hz, 2H), 8.21 (d, J=7.3 Hz), MS 334 (M+H).

(8d) 3-(3,4-Dichlorophenyl)-1-(2-hydroxyethyl)-6-methylpyrimido[5,4-e][1,2,4]triazine-5,7(1H,6H)-dione. 32 mg (58% yield), mp 208-211° C. (dec), ¹H NMR (d-TFA) δ 3.57 (s, 3H), 5.03 (s, 2H), 5.14 (s, 2H), 7.57 (d, J=9.05 Hz, 1H), 8.13 (s, 1H), 8.36 (s, 1H).

(8e) 1-(2-Hydroxyethyl)-3-(4-methoxyphenyl)-6-methylpyrimido[5,4-e][1,2,4]triazine-5,7(1H,6H)-5,7-dione. 26 mg (43% yield), mp 214-215° C. (dec), ¹H NMR (d-DMSO) δ 3.29 (s, 3H), 3.86 (s, 3H), 3.93 (t, J=5.1 Hz, 2H), 4.53 (t, J=5.2 Hz, 2H), 4.98 (t, 1H), 7.16 (d, J=8.5 Hz, 2H), 8.15 (d, J=8.4 Hz, 2H), MS 330.1 (M+H).

(8f) 1-(2-Hydroxyethyl)-6-methyl-3-(4-(2-(pyrrolidin-1-yl)ethoxy)phenyl)pyrimido[5,4-e][1,2,4]triazine-5,7(1H,6H)-dione. 54 mg (28% yield), ¹H NMR (d-DMSO) δ 2.05 (br s, 8H), 3.28 (br s, 5H), 3.68 (br s, 4H), 4.38 (br s, 3H), 7.20 (d, J=5.5 Hz, 2H), 8.36 (d, J=5.5 Hz, 2H), MS 413.2 (M+H).

(8g) 1-(2-Hydroxyethyl)-6-methyl-3-(4-(2-(4-methylpiperazin-1-yl)ethoxy)phenyl)pyrimido[5,4-e][1,2,4]triazine-5,7(1H,6H)-dione. 55 mg (85% yield), ¹H NMR (d-DMSO) δ 2.55 (br s, 8H), 2.90 (s, 3H), 3.29 (s, 3H), 3.95 (t, J=5.45 Hz, 2H), 4.54 (t, J=5.45 Hz, 2H), 7.21 (d, J=8.35 Hz, 2H), 8.18 (d, J=8.4 Hz, 2H), MS 442.1 (M+H).

(8h) 3-(3,4-Dimethoxyphenyl)-1-(2-hydroxyethyl)-6-methylpyrimido[5,4-e][1,2,4]triazine-5,7(1H,6H)-dione. 26 mg (70% yield), mp 215-218° C. (dec), ¹H NMR (d-DMSO) δ 3.29 (s, 3H), 3.87 (s, 6H), 3.94 (s, 2H), 4.54 (s, 2H), 5.00 (t, J=6.15, 1H), 7.18 (d, J=8.35 Hz, 1H), 7.69 (s, 1H), 7.84 (d, J=8.15 Hz, 1H).

### (8i) 3-(4-(Dimethylamino)phenyl)-1-(2-hydroxyethyl)-6-methylpyrimido[5,4-e][1,2,4]triazine-5,7(1H,6H)-dione. 28 mg (71% yield), mp 170-180° C. (dec), ¹H NMR (d-TFA) δ 3.42 (s, 9H), 3.53 (s, 1H), 4.03 (s, 2H), 4.91 (s, 2H), 7.86 (d, J=5.6 Hz, 2H), 8.05 (d, J=5.8 Hz, 2H).

(8j) 1-(2-Hydroxyethyl)-6-methyl-3-(pyridin-2-yl)pyrimido[5,4-e]-[1,2,4]triazine-5,7(1H,6H)-dione. 21 mg (41% yield), ¹H NMR (d-DMSO) δ 3.30 (s, 3H), 3.94 (t, J=4.7 Hz, 2H), 4.57 (t, J=5.0 Hz, 2H), 4.99 (t, J=6.27, 1H), 7.61 (t, J=5.5 Hz, 1H), 8.07 (t, J=7.85 Hz, 1H), 8.25 (d, J=8.05 Hz, 1H), 8.80 (s, 1H).

### Example 4

### Synthesis of Pyrimido[5,4-e][1,2,4]triazine-5,7(1H,6H)-diones (6)

### General Procedure B

An appropriately substituted pyrimidotriazinedione N-oxide (4) (1.5 mmol) in 5 mL dry DMF in an oven-dried flask was stirred under nitrogen and heated to 90° C. for 3-4 hrs. During this time, the mixture darkened and became homogeneous. The mixture then was cooled to room temperature and 6 mL of cold H₂O was added, causing the solution to become cloudy. The mixture was refrigerated overnight, and resulting precipitate was collected by filtration and dried.

The following compounds were prepared according to General Procedure B.

(6a) 6-Methyl-3-p-tolylpyrimido[5,4-e][1,2,4]triazine-5,7(1H,6H)-dione. 59 mg (46% yield), ¹H NMR (d-DMSO) δ2.42 (s, 3H), 3.30 (s, 3H), 7.43 (d, J=7.65 Hz, 2H), 8.31 (d, J=7.85 Hz, 2H).

(6b) 3-(2-Fluorophenyl)-6-methylpyrimido[5,4-e][1,2,4]triazine-5,7(1H,6H)-dione. 198 mg (71% yield), mp 248-250° C., ¹H NMR (d-DMSO) 3.33 (s, 3H), 7.15 (m,

### Example 5

### Synthesis of 8-Alkylpyrimido[5,4-e][1,2,4]triazine-5,7(6H,8H)-diones (7)

### General Procedure C

To an appropriately substituted pyrimidotriazinedione (6) (0.2 mmol) in 2 mL dry acetone in an oven-dried flask under nitrogen was added cesium carbonate (0.3 mmol, 1.5 eq), followed by an appropriate alkyl bromide (0.22 mmol, 1.2 eq). The reaction was stirred at room temperature overnight (benzyl bromides) or heated to 50° C. in a sealed vessel overnight (alkyl and benzyl chlorides). H₂O then was added to the reaction mixture to precipitate the product. The mixture was cooled in a refrigerator for 3-4 hrs, and the resulting precipitate was collected and dried at 65° C./20 mmHg.

The following compounds were prepared according to General Procedure C.

(7a) 3-(4-Chlorophenyl)-6-methyl-8-propylpyrimido[5,4-e][1,2,4]triazine-5,7(6H,8H)-dione. 29 mg (47% yield), mp 202-205° C., ¹H NMR (d-DMSO) δ 0.98 (t, J=7.4 Hz, 3H), 1.76 (m, 2H), 3.37 (s, 3H), 4.32 (t, J=7.2 Hz, 2H), 7.71 (d, J=8.05 Hz, 2H), 8.44 (d, J=7.9 Hz, 2H).

(7b) 3-(4-Fluorophenyl)-6-methyl-8-octylpyrimido[5,4-e][1,2,4]triazine-5,7(6H,8H)-dione. 8 mg (16% yield), ¹H NMR (d-DMSO) δ 0.86 (t, J=6.3 Hz, 3H), 1.33 (m, 8H), 1.39 (m, 2H), 1.73 (t, J=7.3 Hz, 2H), 3.37 (s, 3H), 4.34 (t, J=7.4 Hz, 2H), 7.48 (d, J=8.0 Hz, 2H), 8.49 (d, J=7.6 Hz, 2H).

(7c) 8-(2-(Diethylamino)ethyl)-3-(4-methoxyphenyl)-6-methylpyrimido[5,4-e][1,2,4]triazine-5,7(6H,8H)-dione. 18 mg (22% yield), ¹H NMR (d-DMSO) δ 0.98 (t, J=7.0 Hz, 6H), 2.58 (q, J=7.1 Hz, 4H), 2.73 (t, J=7.0 Hz, 2H), 3.36 (s, 3H), 3.88 (s, 3H), 4.40 (t, J=6.8 Hz, 2H), 7.18 (d, J=8.7 Hz, 2H), 8.38 (d, J=8.5 Hz, 2H).

(7d) 8-Benzyl-3-(2-fluorophenyl)-6-methylpyrimido[5,4-e][1,2,4]triazine-5,7(6H,8H)-dione. 19 mg (35% yield), ¹H NMR (d-DMSO) δ 3.38 (s, 3H), 5.57 (s, 2H), 7.30 (m, 3H), 7.46 (m, 4H), 7.66 (s, 1H), 8.04 (d, J=5.85 Hz, 1H).

(7e) 6-Methyl-8-phenethyl-3-phenylpyrimido[5,4-e][1,2,4]triazine-5,7(6H,8H)-dione. 28 mg (39% yield), ¹H NMR (d-DMSO) 3.25 (s, 2H), 3.45 (s, 3H), 4.57 (s, 2H), 7.26 (s, 1H), 7.36 (s, 4H), 7.65 (s, 3H), 8.46 (s, 2H).

(7f) 6-Methyl-3-phenyl-8-(3-phenylpropyl)pyrimido[5,4-e][1,2,4]triazine-5,7(6H,8H)-dione. 16 mg (22% yield), ¹H NMR (d-DMSO) δ 2.06 (s, 2H), 2.73 (s, 2H), 3.34 (s, 3H), 4.41 (s, 2H), 7.23 (m, 5H), 7.62 (m, 3H), 8.42 (s, 2H).

(7g) 3-(2-Fluorophenyl)-6-methyl-8-(4-methylbenzyppyrimido[5,4-e][1,2,4]triazine-5,7(6H,8H)-dione. 24 mg (58% yield), mp 320° C. (dec), ¹H NMR (d-DMSO) δ 2.27 (s, 3H), 3.39 (s, 3H), 5.53 (s, 1H), 7.14 (d, J=7.65 Hz, 2H), 7.36 (d, J=7.9 Hz, 2H), 7.46 (m, 2H), 7.67 (m, 1H), 8.05 (t, J=8.2 Hz, 1H).

(7h) 8-(4-tert-Butylbenzyl)-3-(4-fluorophenyl)-6-methylpyrimido[5,4-e][1,2,4]triazine-5,7(6H,8H)-dione. 54 mg (71% yield), mp 213-216° C. (dec), ¹H NMR (d-DMSO) δ 1.25 (s, 9H), 3.39 (s, 3H), 5.53 (s, 2H), 7.34 (d, J=7.65 Hz, 2H), 7.40 (d, J=7.8 Hz, 2H), 7.47 (t, J=8.5 Hz, 2H), 8.49 (d, J=6.6 Hz, 2H).

### (7i) 8-(4-Fluorobenzyl)-6-methyl-3-phenylpyrimido[5,4-e][1,2,4]triazine-5,7(6H,8H)-dione. 12 mg (16% yield), mp 253-254° C., ¹H NMR (d-DMSO) δ 3.39 (s, 3H), 5.56 (s, 2H), 7.17 (t. J=8.7 Hz, 2H), 7.52 (d, J=8.4 Hz, 2H), 7.63 (m, 3H), 8.42 (d, J=8.0 Hz).

(7j) 8-(4-Fluorobenzyl)-3-(4-methoxyphenyl)-6-methylpyrimido[5,4-e][1,2,4]triazine-5,7(6H,8H)-dione. 56 mg (81% yield), mp 230-234° C. (dec), ¹H NMR (d-DMSO) δ 3.37 (s, 3H), 3.87 (s, 3H), 5.53 (s, 3H), 7.16 (m, 4H), 7.51 (m, 2H), 8.37 (d, J=8.85 Hz, 2H).

(7k) 8-(3-Fluorobenzyl)-6-methyl-3-phenylpyrimido[5,4-e][1,2,4]triazine-5,7(6H,8H)-dione. 47 mg (66% yield), ¹H NMR (d-DMSO) δ 3.40 (s, 3H), 5.60 (s, 2H), 7.11 (t, J=7.2 Hz, 1H), 7.31 (m, 2H), 7.39 (m, 1H), 7.63 (d, J=2.6 Hz, 3H), 8.43 (m, 2H).

### (7l) 8-(3-Fluorobenzyl)-3-(4-methoxyphenyl)-6-methylpyrimido[5,4-e][1,2,4]triazine-5,7(6H,8H)-dione. 168 mg (80% yield), ¹H NMR (d-DMSO) δ 3.38 (s, 3H), 3.86 (s, 3H), 5.57 (s, 2H), 7.0 (m, 1H), 7.17 (d, J=6.15 Hz, 2H), 7.27 (m, 2H), 7.48 (m, 1H), 8.37 (d, J=6.9 Hz, 2H).

(7m) 8-(3-Fluorobenzyl)-6-methyl-3-(3-(trifluoromethyl)phenyl)pyrimido[5,4-e][1,2,4]triazine-5,7(6H,8H)-dione. 24 mg (33% yield), ¹H NMR (d-DMSO) δ 3.39 (s, 3H), 5.59 (s, 2H), 7.10 (m, 1H), 7.32 (m, 2H), 7.39 (m, 1H), 7.90 (m, 1H), 8.01 (d, J=5.9 Hz, 1H), 8.66 (s, 1H), 8.73 (d, J=6.0 Hz, 1H).

(7n) 3-(4-Chlorophenyl)-8-(2-fluorobenzyl)-6-methylpyrimido[5,4-e][1,2,4]triazine-5,7(6H,8H)-dione. 39 mg (57% yield), mp 251-254° C., ¹H NMR (d-DMSO) δ 3.40 (s, 3H), 5.61 (s, 2H), 7.09 (t, 1H), 7.27 (t, 1H), 7.35 (m, 2H), 7.71 (d, J=8.1 Hz, 2H), 8.44 (d, J=7.9 Hz, 2H).

(7o) 6-Methyl-3-phenyl-8-(4-(trifluoromethyl)benzyl)pyrimido[5,4-e][1,2,4]triazine-5,7(6H,8H)-dione. 32 mg (39% yield), mp 225-227° C. (dec), NMR (d-DMSO) δ 3.47 (s, 3H), 5.66 (s, 2H), 7.16 (t, J=8.7 Hz, 2H), 7.52 (t, J=8.4 Hz, 2H), 7.63 (m, 3H), 8.43 (d, J=4.2 Hz, 2H).

(7p) 8-(3,4-Difluorobenzyl)-1-(4-fluorophenyl)-6-methylpyrimido[5,4-e][1,2,4]triazine-5,7(6H,8H)-dione. 73 mg (91% yield), mp 226° C. (dec), ¹H NMR (d-DMSO) δ 3.38 (s, 3H), 5.55 (s, 2H), 7.34 (s, 1H), 7.42 (m, 4H), 8.47 (d, J=5.6 Hz, 2H).

(7q) 8-(3,4-Difluorobenzyl)-3-(4-hydroxyphenyl)-6-methylpyrimido[5,4-e][1,2,4]triazine-5,7(6H,8H)-dione. 97 mg (91% yield), ¹H NMR (d-DMSO) δ 3.38 (s, 3H), 5.53 (s, 2H), 6.97 (d, J=6.4 Hz, 2H), 7.31 (s, 1H), 7.35 (m, 1H), 7.48 (m, 1H), 8.27 (d, J=6.4 Hz, 2H), 10.19 (s, 1H).

(7r) 8-(2,4-Dichlorobenzyl)-3-(4-methoxyphenyl)-6-methylpyrimido[5,4-e][1,2,4]triazine-5,7(6H,8H)-dione. 6 mg (9% yield), ¹H NMR (d-DMSO) δ 3.83 (s, 3H), 5.52 (s, 2H), 7.16 (d, J=8.15 Hz, 2H), 7.31 (s, 1H), 7.41 (d, J=7.95 Hz, 1H), 7.73 (s, 1H), 8.36 (d, J=8.55 Hz, 2H).

(7s) 6-Methyl-8-(4-nitrobenzyl)-3-phenylpyrimido[5,4-e][1,2,4]triazine-5,7(6H,8H)-dione. 32 mg (42% yield), mp 274-280° C. (dec), ¹H NMR (d-DMSO) δ 3.41 (s, 3H), 5.70 (s, 2H), 7.64 (s, 3H), 7.75 (d, J=8.5 Hz, 2H), 8.20 (d, J=8.6 Hz, 2H), 8.43 (s, 2H).

### Example 6

### Synthesis of Hydroxyphenyl-6-methylpyrimido[5,4-e][1,2,4]triazine-5,7(6H,8H)-diones (9)

### General Procedure D

To a solution of an appropriately substituted 8-alkylpyrimido[5,4-e][1,2,4]triazine-5,7(6H,8H)-dione (7) (0.2 mmol) in 2 mL CH₂Cl₂ at room temperature under nitrogen was added dropwise 0.6 mL (3 eq) of a 1.0M solution of BBr₃ in CH₂Cl₂. The reaction was stirred at room temperature until TLC (5% MeOH/CH₂Cl₂) showed consumption of the starting material (2-48 hrs). Methanol (3-4 mL) was added to the reaction, and the mixture was stirred for an additional 30 minutes. The mixture then was concentrated and triturated in methanol to furnish a bright yellow precipitate. The precipitate was collected, washed with methanol, and dried.

The following compounds were prepared according to General Procedure D.

1. 8-(3,4-Difluorobenzyl)-3-(4-hydroxyphenyl)-6-methylpyrimido[5,4-e][1,2,4]triazine-5,7(6H,8H)-dione (p-OH, R₈=3,4-difluorobenzyl). 97 mg (91% yield), ¹H NMR (d-DMSO) δ 137 (s, 3H), 5.53 (s, 2H), 6.97 (d, J=8.65 Hz, 2H), 7.33 (s, 1H), 7.40 (d, J=8.1 Hz, 1H), 7.50 (d, 0.1=8.1 Hz, 1H), 8.28 (d, J=8.65 Hz, 2H), 10.19 (s, 1H).

2. 8-(3-Fluorobenzyl)-3-(4-hydroxyphenyl)-6-methylpyrimido[5,4-e][1,2,4]triazine-5,7(6H,8H)-dione (p-OH, R₈=3-fluorobenzyl). 35 mg (41% yield), mp 285-288° C. (dec), ¹H NMR (d-DMSO) δ 3.38 (s, 3H), 5.56 (s, 2H), 6.97 (d, J=8.7 Hz, 2H), 7.09 (t, J=8.1 Hz, 1H), 7.27 (s, 1H), 7.30 (d, J=8.7 Hz, 1H), 7.38 (d, J=7.7 Hz, 1H), 10.18 (br s, 1H).

3. 8-(2-Fluorobenzyl)-3-(3-hydroxyphenyl)-6-methylpyrimido[5,4-e][1,2,4]triazine-5,7(6H,8H)-dione (m-OH, R₈=2-fluorobenzyl). 37 mg (52% yield), mp 292-295° C. (dec), ¹H NMR (d-DMSO+CDCl₃) δ 3.77 (s, 3H), 6.00 (s, 2H), 7.25 (d, J=7.5 Hz, 1H), 7.31 (m, 1H), 7.53 (d, J=6.9 Hz, 1H), 7.58 (t, J=7.7 Hz, 2H), 8.00 (s, 1H), 8.22 (d, J=7.7 Hz, 2H), 9.67 (s, 1H).

4. 8-(2-Fluorobenzyl)-3-(4-hydroxyphenyl)-6-methylpyrimido[5,4-e][1,2,4]triazine-5,7(6H,8H)-dione (p-OH, R₈=2-fluorobenzyl). 55 mg (47% yield), ¹H NMR (d-DMSO) δ 3.18 (s, 3H), 5.64 (s, 2H), 6.87 (d, J=8.8 Hz, 2H), 7.02 (m, 2H), 7.25 (m, 2H), 8.27 (d, J=8.8 Hz, 2H), 9.76 (s, 1H).

5. 8-(4-Fluorobenzyl)-3-(4-hydroxyphenyl)-6-methylpyrimido[5,4-e][1,2,4]triazine-5,7(6H,8H)-dione (p-OH, R₈=4-fluorobenzyl). 51 mg (39% yield), mp 333-337° C. (dec), ¹H NMR (d-DMSO) δ 3.40 (s, 3H), 5.52 (s, 2H), 6.97 (d, J=6.4 Hz, 2H), 7.14 (d, J=6.8 Hz, 2H), 7.51 (d, J=6.8 Hz, 2H), 8.27 (d, J=6.4 Hz, 2H), 10.18 (s, 1H).

### Example 7

### Synthesis of (2-(Diethylamino)ethoxy)phenyl)-6-methylpyrimido[5,4-e][1,2,4]triazine-5,7(6H,8H)-diones (10)

### General Procedure E

To a solution of an appropriately substituted hydroxyphenyl-6-methylpyrimido[5,4-e][1,2,4]triazine-5,7(6H,8H)-dione (9) (0.1 mmol) in 1 mL dry acetone in an oven-dried flask under nitrogen was added cesium carbonate (0.22 mmol, 2.2 eq), followed by 2-(diethylamino)ethyl chloride hydrochloride (0.11 mmol, 1.1 eq). The reaction vessel was sealed under nitrogen and heated at 50° C. overnight. The reaction then was cooled to room temperature and filtered. The filtrate was concentrated and triturated in ethanol. The resulting precipitate was collected by filtration and dried.

The following compounds were prepared according to General Procedure E.

(10a) 3-(4-(2-(Diethylamino)ethoxy)phenyl)-8-(4-fluorobenzyl)-6-methylpyrimido[5,4-e][1,2,4]triazine-5,7(6H,8H)-dione. 25 mg (47% yield), ¹H NMR (d-DMSO) 60.99 (t, J=6.9 Hz, 6H), 2.56 (t, J=6.9 Hz, 4H), 2.81 (t, J=5.8 Hz, 2H), 3.33 (s, 3H), 4.12 (d, J=6.0 Hz, 2H), 5.53 (s, 2H), 7.16 (d, J=8.5 Hz, 4H), 7.51 (d, J=8.0 Hz, 2H), 8.35 (d, J=8.65 Hz, 2H).

(10c) 3-(4-(2-(Diethylamino)ethoxy)phenyl)-8-(3-fluorobenzyl)-6-methylpyrimido[5,4-e][1,2,4]triazine-5,7(6H,8H)-dione. 20 mg (45% yield), ¹H NMR (d-DMSO) 80.99 (t, J=6.9 Hz, 6H), 2.57 (t, J=6.9 Hz, 4H), 2.81 (t, J=5.5 Hz, 2H), 3.37 (s, 3H), 4.13 (d, J=5.8 Hz, 2H), 5.57 (s, 2H), 7.08 (t, J=7.10 Hz, 1H), 7.16 (d, J=8.8 Hz, 2H), 7.29 (m, 2H), 7.37 (m, 1H), 8.35 (d, J=6.65 Hz, 2H).

(10d) 3-(3-(2-(Diethylamino)ethoxy)phenyl)-8-(3-fluorobenzyl)-6-methylpyrimido[5,4-e][1,2,4]triazine-5,7(6H,8H)-dione. 28 mg (48% yield), ¹H NMR (d-DMSO) δ 0.99 (t, J=6.9 Hz, 6H), 2.57 (m, 4H), 2.82 (s, 2H), 3.47 (s, 3H), 4.13 (s, 2H), 5.58 (s, 2H), 7.09 (m, 1H), 7.20 (s, 1H), 7.3) (m, 2H), 7.38 (d, J=6.95 Hz, 1H), 7.53 (d, J=7.0 Hz, 1H), 7.93 (s, 1H), 7.99 (d, J=6.8 Hz, 1H).

(10e) 3-(4-(2-(Diethylamino)ethoxy)phenyl)-8-(2-fluorobenzyl)-6-methylpyrimido[5,4-e][1,2,4]triazine-5,7(6H,8H)-dione. 48 mg (80% yield), ¹H NMR (d₆-acetone) δ 1.06 (t, J=5.3 Hz, 6H), 2.67 (m, 4H), 2.94 (s, 2H), 3.47 (s, 3H), 4.22 (s, 2H), 5.76 (s, 2H), 7.06 (m, 1H), 7.14 (m, 3H), 7.32 (m, 1H), 7.41 (m, 1H), 8.45 (d, J=6.1 Hz, 2H).

(10f) 3-(3-(2-(Diethylamino)ethoxy)phenyl)-8-(2-fluorobenzyl)-6-methylpyrimido[5,4-e][1,2,4]triazine-5,7(6H,8H)-dione. 15 on (34% yield), ¹H NMR (d-DMSO) δ 0.99 (t, J=6.9 Hz, 6H), 2.57 (q, J=6.8 Hz, 4H), 2.80 (t, J=6.35 Hz, 2H), 3.38 (s, 3H), 4.10 (t, J=5.5 Hz, 2H), 5.58 (s, 2H), 7.07 (t, J=7.1 Hz, 1H), 7.17 (d, J=8.2 Hz, 1H), 7.24 (t, J=8.5 Hz, 1H), 7.32 (m, 1H), 7.36 (m, 1H), 7.51 (t, J=8.2 Hz, 1H), 7.91 (s, 1H), 7.98

(10g) 3-(4-(2-(Diethylamino)ethoxy)phenyl)-8-(3,4-difluorobenzyl)-6-methylpyrimido[5,4-e][1,2,4]triazine-5,7(6H,8H)-dione. 25 mg (36% yield), ¹H NMR (d-DMSO) δ0.98 (t, J=5.7 Hz, 3H), 2.57 (m, 4H), 2.82 (t, J=4.6 Hz, 2H), 3.38 (s, 3H), 4.11 (d, J=4.6 Hz, 2H), 5.54 (s, 2H), 7.16 (d, J=6.7 Hz, 2H), 7.33 (s, 1H), 7.40 (d, J=8.1 Hz, 1H), 7.51 (d, J=8.1 Hz, 1H), 8.36 (d, J=6.9 Hz, 2H), MS 497.1 (M+H). The hydrochloride salt of (10g) was generated by dissolving the free base in a solution of hydrogen chloride (generated from acetyl chloride in methanol). Upon standing, the product precipitated and was collected. ¹H NMR (CD₃OD) δ 1.42 (t, J=7.3 Hz, 6H), 3.39 (q, J=7.3 Hz, 4H), 3.50 (s, 3H), 3.68 (t, J=4.7 Hz, 2H), 4.50 (t, J=4.8 Hz, 2H), 5.65 (s, 2H), 7.24 (m, 3H), 7.39 (s, 1H), 7.49 (t, J=9.2 Hz, 1H), 8.57 (d, J=8.9 Hz, 2H).

(10h) 3-(3-(2-(Diethylamino)ethoxy)phenyl)-8-(3,4-difluorobenzyl)-6-methylpyrimido[5,4-e][1,2,4]triazine-5,7(6H,8H)-dione. 22 mg (59% yield), ¹H NMR (d-DMSO) δ 0.99 (t, J=6.9 Hz, 6H), 2.56 (m, 4H), 2.81 (t, J=5.5 Hz, 2H), 3.41 (s, 3H), 4.11 (t, J=5.5 Hz, 2H), 5.55 (s, 2H), 7.19 (m, 1H), 7.34 (s, 1H), 7.40 (m, 1H), 7.52 (m, 2H), 7.93 (s, 1H), 8.00 (d, J=6.8 Hz, 1H), ¹⁹F NMR (d-DMSO) −136, −134.

### Example 8

### 1-(Benzyloxymethyl)-6-chloropyrimidine-2,4(1H,3H)-dione (12)

1-(Benzyloxymethyl)-6-chloropyrimidine-2,4(1H,3H)-dione (12) was synthesized from 6-chlorouracil (11) according to method of Edstrom and Wei (J. Org. Chem. 60:5069-5076 (1995)).

### Example 9

### Synthesis of N³-Substituted-1-(Benzyloxymethyl)-6-chloropyrimidinediones (13)

### General Procedure F

To a suspension of 1-(benzyloxymethyl)-6-chloropyrimidine-2,4(1H,3H)-dione (12) (0.627 g, 2.35 mmol) in acetone in an oven-dried flask equipped with a drying tube was added benzyl bromide (2.59 mmol, 1.1 eq), followed by cesium carbonate (1.148 g, 3.53 mmol, 1.5 eq). The reaction was stirred at room temperature overnight. The reaction then was filtered, and the filtrate was concentrated to an oil. Purification by silica gel flash chromatography (25-75% EtOAc/hexanes) furnished the N³-substituted product.

The following compounds were prepared according to General Procedure F.

(13c) 1-(Benzyloxymethyl)-6-chloro-3-(2-hydroxyethyl)pyrimidine-2,4(1H,3H)-dione. 365 mg (92% yield), ¹H NMR (CDCl₃) δ 3.84 (t, J=5.0 Hz, 2H), 4.13 (t, J=5.2 Hz, 2H), 4.70 (s, 2H), 5.60 (s, 2H), 5.95 (s, 1H), 7.35 (s, 5H).

(13d) 1-(Benzyloxymethyl)-6-chloro-3-(4-fluorobenzyl)pyrimidine-2,4(1H,3H)-dione. 453 g 100% yield), ¹H NMR (CDCl₃) δ 4.68 (s, 2H), 5.01 (s, 2H), 5.57 (s, 2H), 5.93 (s, 1H), 7.02 (d, J=8.6 Hz, 2H), 7.31 (m, 5H), 7.47 (d, J=8.6 Hz, 2H).

(13e) 1-(Benzyloxymethyl)-6-chloro-3-(3,4-difluorobenzyl)pyrimidine-2,4(1H,3H)-dione. 431 mg (98% yield), ¹H NMR (CDCl₃) δ 4.69 (s, 2H), 4.97 (s, 2H), 5.57 (s, 2H), 5.96 (s, 1H), 7.28 (m, 8H).

### Example 10

### Synthesis of Hydrazinylpyrimidine-2,4(1H,3H)-diones (14)

The following compounds were prepared from an appropriate N³-substituted-1-(benzyloxymethyl)-6-chloropyrimidinedione (13) according to the method of Daves et al. (Journal of the American Chemical Society 84:1724-1729 (1962)).

(14c) 1-(Benzyloxymethyl)-3-(2-hydroxyethyl)-6-(1-methylhydrazinyl)pyrimidine-2,4(1H,3H)-dione. 441 mg (44% yield), ¹H NMR (d-DMSO) δ 2.89 (s, 3H), 3.46 (t, J=6.1 Hz, 2H), 3.83 (t, J=6.1 Hz, 2H), 4.58 (s, 2H), 5.13 (s, 1H), 5.56 (s, 2H), 7.26 (m, 2H), 7.32 (m, 3H).

(14d) 1-(Benzyloxymethyl)-3-(4-fluorobenzyl)-6-(1-methylhydrazinyl)pyrimidine-2,4(1H,3H)-dione. 188 mg (51% yield), ¹H NMR (d-DMSO) 2.86 (s, 3H), 4.74 (s, 2H), 4.94 (s, 2H), 5.17 (s, 1H), 5.58 (s, 2H), 7.15 (d, J=8.8 Hz), 7.27 (m, 7H).

### Example 11

### Synthesis of Hydrazones (15)

The following compounds were synthesized from an appropriately substituted hydrazinylpyrimidine-2,4(1H,3H)-dione (14) according to the method of Nagamatsu, et al. (Chemical & Pharmaceutical Bulletin 41:362-368 (1993)).

(15c) 1-(Benzyloxymethyl)-3-(2-hydroxyethyl)-6-(2-(4-methoxybenzylidene)-1-methylhydrazinyl)pyrimidine-2,4(1H,3H)-dione. 150 mg (50% yield), ¹H NMR (d-DMSO) δ 3.33 (s, 3H), 3.45 (t, J=6.1 Hz, 2H), 3.51 (t, J=6.2 Hz, 2H), 3.78 (s, 3H), 3.88 (s, 2H), 4.60 (s, 2H), 5.49 (s, 1H), 6.93 (d, J=7.7 Hz, 2H), 7.28 (m, 5H), 7.61 (d, J=7.7 Hz, 2H), 7.85 (s, 1H).

(15d) 1-(Benzyloxymethyl)-3-(4-fluorobenzyl)-6-(2-(4-methoxybenzylidene)-1-methylhydrazinyl)pyrimidine-2,4(1H,3H)-dione. 92 mg (25% yield), mp 170-173° C., ¹H NMR (d-DMSO) δ 3.21 (s, 3H), 3.77 (s, 3H), 4.56 (s, 2H), 4.96 (s, 2H), 5.43 (s, 1H), 5.50 (s, 2H), 6.92 (d, J=8.7 Hz, 2H), 7.16 (t, J=8.8 Hz, 2H), 7.22 (m, 5H), 7.29 (t, J=8.4 Hz, 2H), 7.60 (d, J=8.6 Hz, 2H), 7.85 (s, 1H).

(15e) 1-(Benzyloxymethyl)-3-(3,4-difluorobenzyl)-6-(2-(2-fluorobenzylidene)-1-methylhydrazinyl)pyrimidine-2,4(1H,3H)-dione. 413 mg (47% yield), ¹H NMR (d-DMSO) δ 3.28 (s, 3H), 4.68 (s, 2H), 5.06 (s, 2H), 5.45 (s, 1H), 5.60 (s, 2H), 7.10 (d, J=8.7 Hz, 2H), 7.29 (m, 8H), 7.85 (d, J=8.8 Hz, 2H), 8.04 (s, 1H).

### Example 12

### Synthesis of Pyrimido[5,4-e][1,2,4]triazine-5,7(1H,6H)-diones (17)

The following compounds were prepared from an appropriately substituted hydrazone (15) according to the general procedure provided above for Example 3.

(17a) 3-(2-Fluorophenyl)-1-methylpyrimido[5,4-e][1,2,4]triazine-5,7(1H,6H)-dione. 184 mg (68% yield), mp 240-247° C. (dec), ¹H NMR (d-DMSO) δ 4.33 (s, 3H), 7.43 (s, 2H), 7.79 (s, 1H), 8.16 (s, 1H), 11.61 (br s, 1H).

(17b) 3-(2,4-Dichlorophenyl)-1-methylpyrimido[5,4-e][1,2,4]triazine-5,7(1H,6H)-dione. 1.0 g (72% yield), ¹H NMR (d-TFA) δ 4.43 (s, 3H), 7.40 (s, 1H), 7.56 (s, 1H), 7.79 (s, 1H), MS 324.0, 326.0 (M+H).

(17c) 6-(2-Hydroxyethyl)-3-(4-methoxyphenyl)-1-methylpyrimido[5,4-e][1,2,4]triazine-5,7(1H,6H)-dione. 30 mg (10% yield), ¹H NMR (d-DMSO) δ 3.56 (t, J=6.3 Hz, 2H), 3.83 (s, 3H), 3.87 (t, J=6.3 Hz, 2H), 4.04 (s, 3H), 4.79 (br s, 1H), 7.15 (d, J=8.6 Hz, 2H), 8.15 (d, J=8.1 Hz, 2H), MS 352.1 (M+Na).

(17d) 6-(4-Fluorobenzyl)-3-(4-methoxyphenyl)-1-methylpyrimido[5,4-e][1,2,4]triazine-5,7(1H,6H)-dione. 11 mg (36% yield), ¹H NMR (CDCl₃) δ 3.85 (s, 3H), 4.37 (s, 3H), 5.40 (s, 2H), 7.17 (d, J=8.7 Hz, 4H), 7.43 (d, J=5.85 Hz, 2H), 8.15 (d, J=12.3 Hz, 2H), MS 394.1 (M+H).

(17e) 6-(3,4-Difluorobenzyl)-3-(2-fluorophenyl)-1-methylpyrimido[5,4-e][1,2,4]triazine-5,7(1H,6H)-dione. 64 mg (66% yield), mp 200-202° C. (dec), ¹H NMR (d-DMSO) δ 64.06 (s, 3H), 5.08 (s, 2H), 7.38 (s, 1H), 7.45 (m, 4H), 7.65 (br s, 1H), 8.00 (s, 1H), MS 400.1 (M+H).

Tables 1 and 2 list representative compounds prepared in Examples 1 to 12.

TABLE 1
Substituted Pyrimido[5,4-e][1,2,4]triazine-5,7(1H,6H)-diones

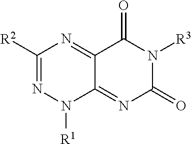

| Compound | R¹ | R² | R³ |
|---|---|---|---|
| 6a¹ | H | 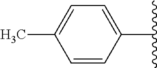 | CH₃ |
| 6b | H | 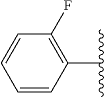 | CH₃ |
| 5a²⁻⁴ | CH₃ | H | CH₃ |
| 5b²⁻⁴ | CH₃ | CH₃ | CH₃ |
| 5c | CH₃ | CH₂CH₃ | CH₃ |
| 5d^{2,5,6} | CH₃ | 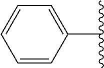 | CH₃ |
| 5e | CH₃ | 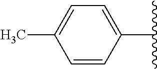 | CH₃ |
| 5f | CH₃ | 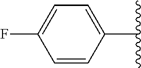 | CH₃ |
| 5g | CH₃ | 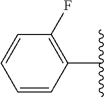 | CH₃ |
| 5h^{2,5} | CH₃ | 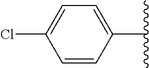 | CH₃ |
| 5i⁷ | CH₃ | 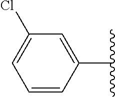 | CH₃ |
| 5j² | CH₃ | 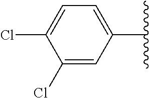 | CH₃ |
| 5k^{2,5} | CH₃ | 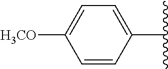 | CH₃ |
| 5l | CH₃ | 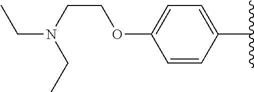 | CH₃ |
| 5m | CH₃ | 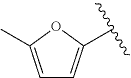 | CH₃ |
| 5n^{2,5} | CH₃ | 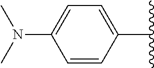 | CH₃ |
| 5o | CH₃ | 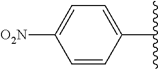 | CH₃ |
| 5p | CH₃ | 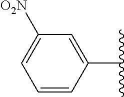 | CH₃ |
| 5q^{2,3} | CH₃ | 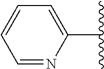 | CH₃ |
| 8a | CH₂CH₂OH | 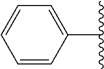 | CH₃ |
| 8b | CH₂CH₂OH | 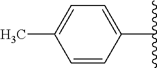 | CH₃ |
| 8c | CH₂CH₂OH | 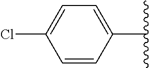 | CH₃ |
| 8d | CH₂CH₂OH | 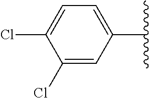 | CH₃ |
| 8e | CH₂CH₂OH | 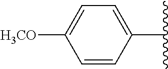 | CH₃ |
| 8f | CH₂CH₂OH | 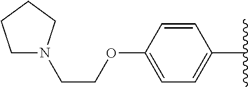 | CH₃ |
| 8g | CH₂CH₂OH | 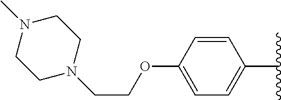 | CH₃ |
| 8h | CH₂CH₂OH | 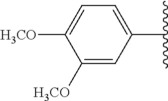 | CH₃ |
| 8i | CH₂CH₂OH | 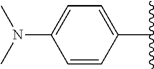 | CH₃ |
| 8j | CH₂CH₂OH | 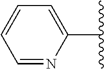 | CH₃ |
| 17a | CH₃ | 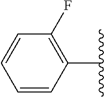 | H |
| 17b | CH₃ | 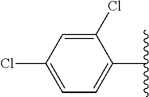 | H |
| 17c | CH₃ | 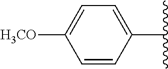 | CH₂CH₂OH |
| 17d | CH₃ | 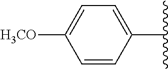 | 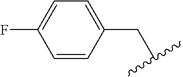 |
| 17e | CH₃ | 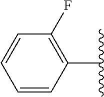 | 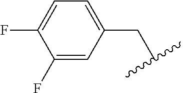 |

¹Yoneda, F. et al. Chemical & Pharmaceutical Bulletin 26: 3154-60 (1978).
²Yoneda, F. et al. Chemical & Pharmaceutical Bulletin 23: 2001-2009 (1975).
³Yoneda, F. et al. Tetrahedron Letters (13): 851-544 (1971).
⁴Daves, G. et al. Journal of the American Chemical Society 84: 1724-1729 (1962)
⁵(a) Yoneda, F. et al. Journal of the Chemical Society, Perkin Transactions 1: Organic and Bio-Organic Chemistry 22: 2398-2402 (1976). (b) Yoneda, F. et al. Synthesis 3: 177-179 (1975).
⁶Nagamatsu, T. JP 07041479.
⁷Middleton, T. et al. Letters in Drug Design & Discovery 4: 1-8 (2007).

TABLE 2
Substituted Pyrimido[5,4-e][1,2,4]triazine-5,7(6H,8H)-diones

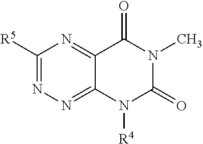

| Compound | R⁴ | R⁵ |
|---|---|---|
| 7a⁸ | CH₂CH₂CH₃ | 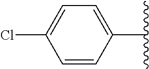 |
| 7b | (CH₂)₇CH₃ | 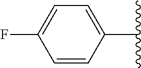 |
| 7c | CH₂CH₂NEt₂ | 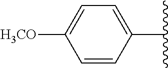 |
| 7d | 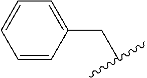 | 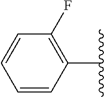 |
| 7e | 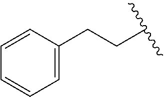 | 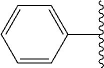 |
| 7f | 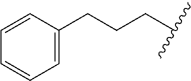 | 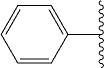 |
| 7g | 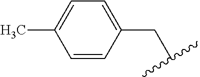 | 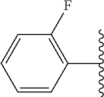 |
| 7h | 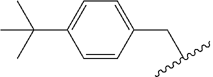 | 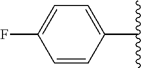 |
| 7i | 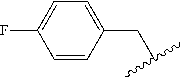 | 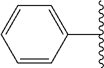 |
| 7j | 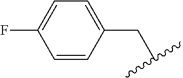 | 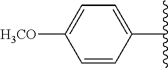 |
| 10a | 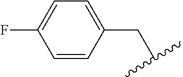 | 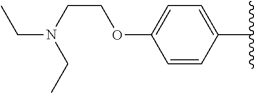 |
| 10b | 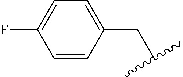 | 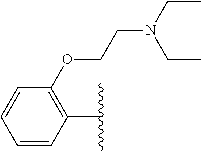 |
| 7k | 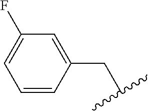 | 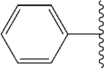 |
| 7l | 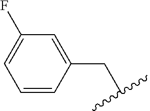 | 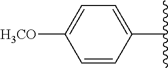 |
| 10c | 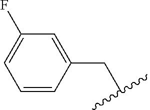 | 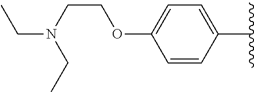 |
| 10d | 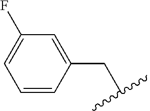 | 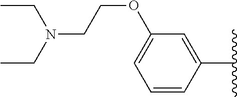 |
| 7m | 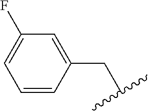 | 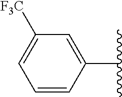 |
| 7n | 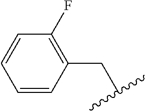 | 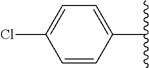 |
| 10e | 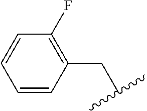 | 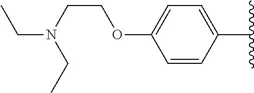 |
| 10f | 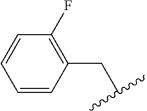 | 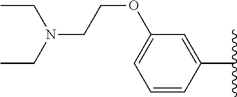 |
| 7o | 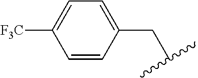 | 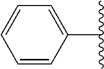 |
| 7p | 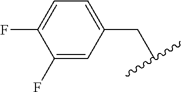 | 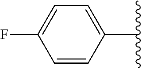 |
| 7q | 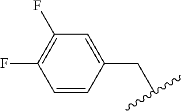 | 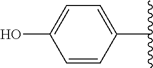 |
| 10g | 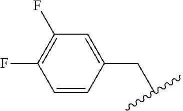 | 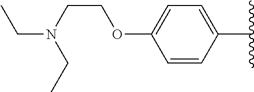 |
| 10g mono- HCl salt | 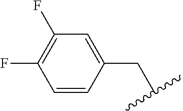 | 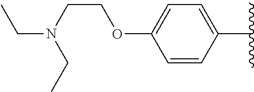 |
| 10h | 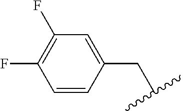 | 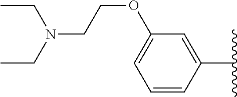 |
| 7r | 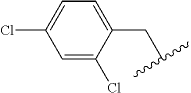 | 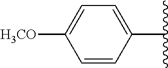 |
| 7s | 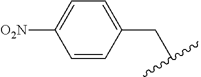 | 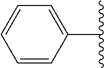 |

⁸Yoneda, F. et al. Journal of Heterocyclic Chemistry 17: 869-871 (1980).

### Example 13

### Measurement of IC₅₀ and LD₅₀ Values for the Substituted Pyrimidotriazinediones

The activity of substituted pyrimidotriazinediones in antagonizing the β-catenin/TCF complex was measured using a cell-based assay system. The non-transformed rat ileal epithelial cell line IEC18 (Cat. No. CRL-1589, available from American Type Tissue Cultures, Inc., Mannassas, Va.) was used because it possesses several characteristics of normal intestinal epithelial cells and shows virtually no baseline transcriptional activation of β-catenin target genes (Quaroni, A. et al. “Epithelioid cell cultures from rat small intestine. Characterization by morphologic and immunologic criteria,” Journal of Cell Biology 80:248-265 (1979)). IEC18 cells were transduced with a retrovirus driving expression of an activated form of β-catenin (the S33Y mutant) and a stable polyclonal cell line was selected using the antibiotic G418 according to the procedure of F. T. Kolligs et al. (“Neoplastic transformation of RK3E by mutant beta-catenin requires deregulation of Tcf/Lef transcription but not activation of c-myc expression,” Molecular and Cellular Biology 19:5696-5706 (1999)), generating the IEC18-S33Y cell line. The S33Y mutant of β-catenin was used because it has an extended in vivo half-life and is capable of translocating to the nucleus to bind and activate transcription factors of the TCF/LEF family in the absence of Wnt signaling.

As a negative control, IEC18 cells were transduced with the empty retroviral backbone lacking the β-catenin expression cassette and a stable polyclonal cell line was selected, generating the IEC18-NEO cell line.

As a control for the general transcriptional status of the cell lines, a lentiviral construct was generated which drives constitutive expression of the Renilla luciferase gene under the control of the CMV promoter. The construct was produced by subcloning the open reading frame of the Renilla luciferase gene (obtained from the pGL4.73 vector, available from Promega, Madison, Wis.) behind the CMV promoter of a modified version of the LL3.7 lentiviral vector (Rubinson, D. A. et al. “A lentivirus-based system to functionally silence genes in primary mammalian cells, stem cells and transgenic mice by RNA interference,” Nature Genetics 33:401-406 (2003)).

To measure β-catenin-mediated transcriptional activity, the β-catenin-dependent firefly luciferase expression cassette was subcloned from the TOPFLASH vector (available from Millipore, Billerica, Mass.) into a self-inactivating lentiviral vector based on the LL3.7 lentiviral vector (Rubinson, D. A. et al. “A lentivirus-based system to functionally silence genes in primary mammalian cells, stem cells and transgenic mice by RNA interference,” Nature Genetics 33:401-406 (2003), Korinek, V. et al. “Constitutive transcriptional activation by a beta-catenin-Tcf complex in APC−/− colon carcinoma,” Science 275:1784-1787 (1997)).

To validate the assay system, IEC18-S33Y and IEC18-NEO cells were infected in parallel with the lentiviral CMV-Renilla construct and the lentiviral β-catenin-dependent firefly luciferase construct. The infections were performed in the presence of 4 μg/mL POLYBRENE hexadimethrine bromide (available from Sigma-Aldrich, St. Louis, Mo.). The lentivirus used for the infections was produced separately for each construct by transient cotransfection of 293T cells with the lentiviral vector and the packaging plasmids pMDLg/pRRE (containing the viral gag/pol elements), pRSV-REV (driving expression of rev), and pMD.G (driving expression of the VSVG envelope protein) (see Dull, T. et al. “A third-generation lentivirus vector with a conditional packaging system,” Journal of Virology 72:8463-8471 (1998)). The infected cells were plated in 96-well plates at several cell concentrations varying from 3125 cells/well to 100,000 cells/well. Transcriptional activity of the firefly and Renilla luciferase genes was determined 48 h after plating by measuring luminescence using the Promega Dual Luciferase kit (available from Promega, Madison, Wis.). The β-catenin-dependent transcriptional activity in the IEC18-S33Y cell line was found to be more than 14-fold increased, as compared to the IEC18-NEO cell line, when cells were plated at a concentration of at least 25,000 cells/well (data not shown).

Using the above assay system, IC₅₀ and LD₅₀ values were determined for the pyrimidotriazinedione compounds. IEC18-S33Y cells carrying a β-catenin firefly luciferase cassette and a constitutively active renilla luciferase cassette were seeded at 40,000 cells/well in 96-well tissue culture plates (BD Biosciences, Bedford, Mass.) in a total volume of 80 μL of tissue culture medium (Dulbecco's modified Eagle medium without Phenol-Red (Invitrogen, Carlsbad, Calif.) supplemented with 10% (v/v) fetal bovine serum (Hyclone, Logan, Utah)) and incubated for 24 h. Stock solutions of the compounds were prepared by dissolving the compounds in DMSO at concentrations ranging from 50 mM to 5 mM, depending on individual solubility. Working solutions of the compounds were obtained by further diluting the stock solutions in tissue culture medium by at least 50-fold. Serial twofold dilutions of the working solutions of the compounds were added to the cells in a volume of 40 μL and the cells were incubated in the presence of the compounds for 17-20 h.

LD₅₀ values of the pyrimidotriazinediones then were determined using a WST-1 colorimetric cell proliferation assay. A mixture containing 20 μL of medium and 10 μL of WST-1 reagent (available from Roche, Indianapolis, Ind.) was added to the cells and the cells were incubated for 1 h at 37° C., after which time absorption at 550 nm was measured using a microplate reader.

To determine IC₅₀ values of the pyrimidotriazinediones, expression of Renilla and firefly luciferase was measured. Following the absorptions measurements, the tissue culture medium was aspirated and the cells were lysed using 100 μL of passive lysis buffer (available from Promega, Madison, Wis.). Renilla and firefly luciferase activity then was measured using the Promega Dual Luciferase kit.

IC₅₀ and LD₅₀ values for pyrimidotriazinedione compounds are listed in Tables 3 and 4, and were calculated using Microcal ORIGIN 7.5 software by fitting sigmoidal curves to the average data for a minimum of two replicates. IC₅₀ and LD₅₀ values for several N-oxide intermediates also were determined, and are provided in Table 5.

TABLE 3
IC₅₀ and LD₅₀ Values for Substituted Pyrimido[5,4-e][1,2,4]triazine-5,7(1H,6H)-diones in IEC18 Rat Epithelial Cells

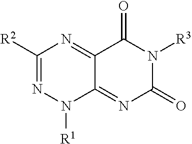

| Compound | R¹ | R² | R³ | LD₅₀ (μM) | IC₅₀ (μM) | LD₅₀/IC₅₀ |
|---|---|---|---|---|---|---|
| 6a¹ | H | 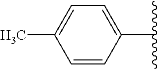 | CH₃ | >50 | 21.08 ± 14.14 | >2.37 |
| 5a²⁻⁴ | CH₃ | H | CH₃ | 0.19 ± 0.01 | 0.11 ± 0.21 | 1.81 |
| 5b²⁻⁴ | CH₃ | CH₃ | CH₃ | 2.02 ± 0.75 | 1.00 ± 0.19 | 2.02 |
| 5c | CH₃ | CH₂CH₃ | CH₃ | 2.94 ± 10 | 1.42 ± 0.25 | 2.07 |
| 5d^{2,5,6} | CH₃ | 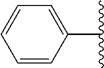 | CH₃ | 1.39 ± 0.44 | 0.53 ± 0.10 | 2.62 |
| 5e | CH₃ | 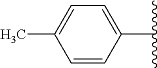 | CH₃ | 0.84 ± 0.25 | 0.68 ± 0.09 | 1.23 |
| 5f | CH₃ | 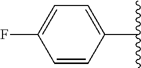 | CH₃ | 1.10 ± 0.01 | 0.30 ± 0.07 | 3.67 |
| 5g | CH₃ | 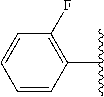 | CH₃ | 0.71 ± 3.31 | 0.19 ± 0.014 | 3.64 |
| 5h^{2,5} | CH₃ | 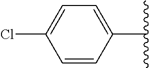 | CH₃ | 2.84 ± 0.80 | 1.05 ± 0.29 | 2.70 |
| 5i⁷ | CH₃ | 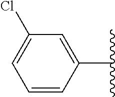 | CH₃ | 23.88 ± 5.17 | 5.03 ± 1.18 | 4.75 |
| 5j² | CH₃ | 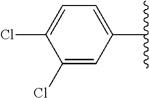 | CH₃ | 30.1 ± 17.54 | 9.32 ± 7.44 | 3.23 |
| 5l | CH₃ | 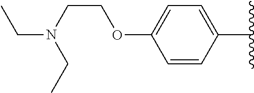 | CH₃ | 0.136 ± 1 | 0.016 ± 0.018 | 8.32 |
| 8a | CH₂CH₂OH | 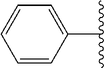 | CH₃ | 2.09 ± 0.78 | 0.97 ± 0.22 | 2.16 |
| 8b | CH₂CH₂OH | 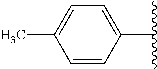 | CH₃ | 2.55 ± 21.76 | 0.32 ± 0.23 | 7.94 |
| 8c | CH₂CH₂OH | 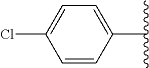 | CH₃ | 6.36 ± 21.76 | 4.56 ± 8.24 | 1.39 |
| 8e | CH₂CH₂OH | 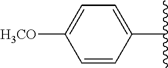 | CH₃ | 8.39 ± 10.29 | 6.07 ± 4.91 | 1.38 |
| 8f | CH₂CH₂OH | 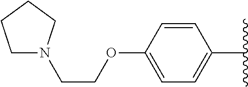 | CH₃ | 1.55 ± 0.22 | 1.34 ± 0.15 | 1.16 |
| 8j | CH₂CH₂OH | 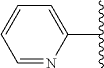 | CH₃ | 9.55 ± 0.001 | 3.81 ± 0.59 | 2.51 |
| 17e | CH₃ | 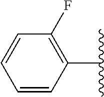 | 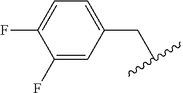 | 0.48 ± 0.10 | 0.11 ± 0.06 | 4.36 |

¹Yoneda, F. et al. Chemical & Pharmaceutical Bulletin 26: 3154-60 (1978).
²Yoneda, F. et al. Chemical & Pharmaceutical Bulletin 23: 2001-2009 (1975).
³Yoneda, F. et al. Tetrahedron Letters (13): 851-544 (1971).
⁴Daves, G. et al. Journal of the American Chemical Society 84: 1724-1729 (1962)
⁵(a) Yoneda, F. et al. Journal of the Chemical Society, Perkin Transactions 1: Organic and Bio-Organic Chemistry 22: 2398-2402 (1976). (b) Yoneda, F. et al. Synthesis 3: 177-179 (1975).
⁶Nagamatsu, T. JP 07041479.
⁷Middleton, T. et al. Letters in Drug Design & Discovery 4: 1-8 (2007).

TABLE 4
IC₅₀ and LD₅₀ Values for Substituted Pyrimido[5,4-e][1,2,4]triazine-5,7(6H,8H)-dione in IEC18 Rat Epithelial Cells

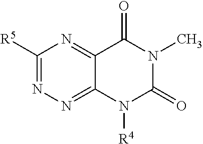

| Compound | R⁴ | R⁵ | LD₅₀ (μM) | IC₅₀ (μM) | LD₅₀/IC₅₀ |
|---|---|---|---|---|---|
| 7d | 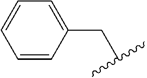 | 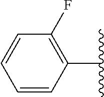 | >50 | 3.60 ± 2.25 | >13.59 |
| 7h | 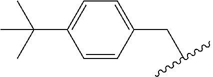 | 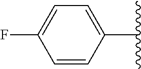 | >300 | 0.33 ± 0.01 | >909 |
| 10c | 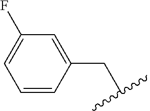 | 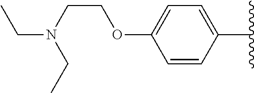 | 21.49 ± 10.42 | 23.41 ± 2.83 | 0.92 |
| 7p | 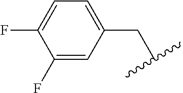 | 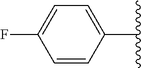 | >50 | 1.68 ± 0.91 | >29.8 |
| 10g | 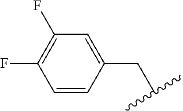 | 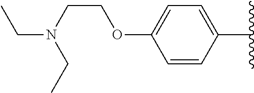 | 2.57 ± 10.99 | 0.70 ± 0.13 | 3.67 |
| 10 g mono- HCl salt | 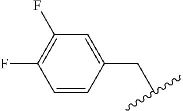 | 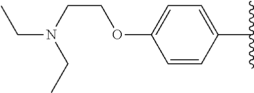 | 2.04 ± 0.76 | 1.10 ± 0.11 | 1.85 |

TABLE 5
IC₅₀ and LD₅₀ Values for N-Oxide Intermediates in IEC18 Rat Epithelial
Cells

| Compound | LD₅₀ (μM) | IC₅₀ (μM) | LD₅₀/IC₅₀ |
|---|---|---|---|
| 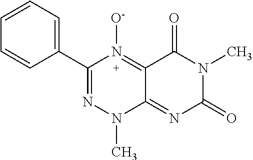 | 1.71 ± 46.77 | 0.49 ± 0.22 | 3.49 |
| 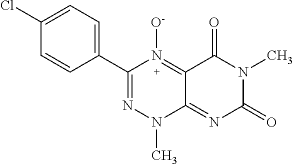 | 2.01 ± 2.58 | 0.70 ± 0.15 | 2.87 |

As shown in Table 3, several compounds of formula I having a methyl group for R³ demonstrated strong inhibitory activity of β-catenin-mediated transcription. Additionally, a compound having a difluorobenzyl group for R³ (17e) also antagonized β-catenin-mediated transcription. The compounds of formula I provided in Table 3 demonstrated IC₅₀ values of less than about 30 μM. Overall, the IC₅₀ values ranged from about 0.001 μM to about 30 μM. More specifically, the IC₅₀ values for the strongest inhibitors ranged from about 0.010 μM to about 10 μM, for example, from about 0.010 μM to about 2 μM.

One of the strongest inhibitors of the β-catenin/Tcf complex from the compound (5) and compound (8) series was (5l) (3-(4-(2-(diethylamino)ethoxy)phenyl)-1,6-dimethylpyrimido[5,4-e][1,2,4]triazine-5,7(1H,6H)-dione), which demonstrated an IC₅₀ of 0.016±0.018 μM. In comparison, the IC₅₀ of the β-catenin/Tcf4 antagonist identified by Lepourcelet et al. (Cancer Cell, 5:91-102 (2004)) was found to be 0.11±0.21 μM (compound (5a)). Advantageously, the LD₅₀/IC₅₀ ratio was significantly improved for compound (5l) compared to compound (5a) (8.32 compared to 1.81).

Additionally, as shown in Table 4, several compounds of formula II having a methyl group for R⁶ demonstrated strong inhibitory activity of β-catenin-mediated transcription. The compounds provided in Table 4 have benzyl and substituted benzyl substituents for R⁴ and substituted phenyl substituents for R⁵. The compounds of formula II provided in Table 4 demonstrated IC₅₀ values of less than about 30 μM. Overall, the IC₅₀ values ranged from about 0.1 μM to about 30 μM. More specifically, the IC₅₀ values for the strongest inhibitors ranged from about 0.1 μM to about 10 μM, for example, from about 0.1 μM to about 2 μM. Advantageously, several compounds (7d, 7h, and 7p) demonstrated LD₅₀ values greater than about 50 μM.

### Example 14

### Pharmacokinetic Properties of Pyrimidotriazinediones in Mice

The pharmacokinetic properties of a substituted pyrimido[5,4-e][1,2,4]triazine-5,7(6H,8H)-dione of formula II (3-(4-(2-(Diethylamino)ethoxy)phenyl)-8-(3,4-difluorobenzyl)-6-methylpyrimido[5,4-e][1,2,4]triazine-5,7(6H,8H)-dione) in mice were determined by dosing a cohort of mice with 10 mg/kg compound. Blood was drawn from the mice at various time points (1 minute, 15 minutes, 30 minutes, 3 hours, 6 hours, and 12 hours) post-injection. The blood plasma was isolated from the drawn blood and the amount of compound present in the plasma was measured by HPLC with peak analysis by mass spectrometry. The substituted pyrimido[5,4-e][1,2,4]triazine-5,7(6H,8H)-dione of formula II demonstrated favorable pharmacokinetic properties as assessed by the peak plasma level and half-life of the compound.

What is claimed is:

1. A method of treating cancer comprising administering a compound haying a formula II, or a pharmaceutically acceptable salt or hydrate thereof: 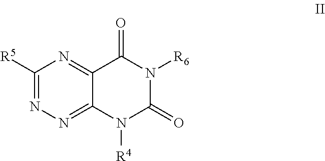
wherein R⁴ and R⁶ are each independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl; and
R⁵ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, optionally substituted heteroaryl, optionally substituted C₁₋₂₀ alkylamino, optionally substituted C₁₋₂₀ alkoxy, and optionally substituted C₁₋₂₀ alkylcarboxamido.

2. The method of claim 1, wherein R⁴ is selected from the group consisting of hydrogen, C₁₋₂₀ alkyl, C₁₋₂₀ alkylene-OH, C₁₋₂₀ alkylene-NR^{a}R^{b}, optionally substituted C₁₋₂₀ alkylene-aryl, and optionally substituted C₁₋₂₀ alkylene-heteroaryl, wherein R^{a} and R^{b} are each independently selected from the group consisting of hydrogen and C₁₋₂₀ alkyl or R^{a} and R^{b} taken together with the nitrogen atom to which they are bound form an optionally substituted 3 to 10 membered heterocyclic ring.

3. The method of claim 1, wherein R⁴ is selected from the group consisting of methyl, ethyl, propyl, octyl, 2-(N,N-diethylamino)ethyl, phenyl, fluorophenyl, cyclopentyl, cyclopropyl, optionally substituted C₁₋₃ alkylene-aryl, and optionally substituted C₁₋₃ alkylene-heteroaryl.

4. The method of claim 1, wherein R⁴ is selected from the group consisting of benzyl, 2-phenethyl, 3-phenpropyl, methylbenzyl, t-butylbenzyl, fluorobenzyl, difluorobenzyl, dichlorobenzyl, nitrobenzyl, trifluoromethylbenzyl, and pyridine-3-ylmethyl.

5. The method of claim 1, wherein R⁵ is selected from the group consisting of optionally substituted C₁₋₂₀ alkylene-aryl, R^{d}C(═O)NR^{c}(C₁₋₂₀ alkyl), optionally substituted R^{e}R^{f}N—(C₁₋₂₀ alkylcarboxamido), optionally substituted (C₁₋₂₀ alkylcarboxamido)phenyl, optionally substituted R^{e}R^{f}N—(C₁₋₂₀ alkylamino), optionally substituted R^{e}R^{f}N—(C₁₋₂₀ alkoxy), optionally substituted C₁₋₂₀ alkylene-NR^{e}R^{f}, optionally substituted C₁₋₂₀ alkoxyphenyl, and optionally substituted C₁₋₂₀ alkylaminophenyl, wherein R^{c} is selected from the group consisting of hydrogen and C₁₋₂₀ alkyl, R^{d} is selected from the group consisting of hydrogen, C₁₋₂₀ alkyl, and optionally substituted C₁₋₂₀ aminoalkyl, and R^{e} and R^{f} are independently selected from the group consisting of hydrogen, optionally substituted C₁₋₂₀ alkyl, and optionally substituted C₁₋₂₀ aminoalkyl, or R^{e} and R^{f} taken together with the nitrogen atom to which they are bound form an optionally substituted 3 to 10 membered heterocyclic ring.

6. The method of claim 1, wherein R⁵ is selected from the group consisting of optionally substituted C₁₋₃ alkylene-aryl, R^{d}C(═O)NR^{c}(C₁₋₃ alkyl), optionally substituted R^{e}R^{f}N—(C₁₋₃ alkylcarboxamido), optionally substituted (C₁₋₃ alkylcarboxamido)phenyl, optionally substituted R^{e}R^{f}N—(C₁₋₃ alkylamino), optionally substituted R^{e}R^{f}N—(C₁₋₃ alkoxy), optionally substituted C₁₋₃ alkylene-NR^{e}R^{f}, optionally substituted C₁₋₃ alkoxyphenyl, and optionally substituted C₁₋₃ alkylaminophenyl, wherein R^{c} is selected from the group consisting of hydrogen and C₁₋₃ alkyl, R^{d} is selected from the group consisting of hydrogen, C₁₋₃ alkyl, and optionally substituted C₁₋₃ aminoalkyl, and R^{e} and R^{f} are independently selected from the group consisting of hydrogen, optionally substituted C₁₋₃ alkyl, and optionally substituted C₁₋₃ aminoalkyl, or R^{e} and R^{f} taken together with the nitrogen atom to which they are bound form an optionally substituted 3 to 6 membered heterocyclic ring.

7. The method of claim 1, wherein R⁵ is selected from the group consisting of optionally substituted C₁₋₃ alkylene-aryl-NR^{e}R^{f}, R^{e}R^{f}N—R^{d}C(═O)NR^{c}(C₁₋₃ alkyl), optionally substituted R^{e}R^{f}N—(C₁₋₃ alkylcarboxamido)phenyl, optionally substituted R^{e}R^{f}N—(C₁₋₃ alkoxy)phenyl, and optionally substituted R^{e}R^{f}N—(C₁₋₃ alkylamino)phenyl, wherein R^{c} is selected from the group consisting of hydrogen and C₁₋₃ alkyl, R^{d} is optionally substituted C₁₋₃ alkyl, and R^{e} and R^{f} are independently selected from the group consisting of hydrogen, optionally substituted C₁₋₃ alkyl, and optionally substituted C₁₋₃ aminoalkyl, or R^{e} and R^{f} taken together with the nitrogen atom to which they are bound form an optionally substituted 3 to 6 membered heterocyclic ring.

8. The method of claim 1, wherein R⁵ is selected from the group consisting of pyridinyl, phenyl, fluorophenyl, chlorophenyl, hydroxyphenyl, methoxyphenyl, trifluoromethylphenyl, carboxyphenyl, (2-(4-aminoacetylpiperazin-1-yl)ethoxy)phenyl, (2-(4-(dimethylamino)piperidin-1-yl)ethoxy)phenyl, (2-(3-aminopyrrolidin-1-yl)ethoxy)phenyl, (3-(morpholin-4-yl)propyloxy)phenyl, (2-(piperidin-1-yl)ethoxy)phenyl, (2-(dimethylamino)ethyl)phenyl, (2-(piperidin-1-yl)ethylamino)phenyl, (2-(diethylamino)ethylcarboxamide)phenyl, (2-(4-methylpiperazin-1-yl)ethoxy)phenyl, (3-(4-methylpiperazin-1-yl)propyl)phenyl, 2-(N,N-diethylamino)ethoxy)phenyl, (2-(morpholin-4-yl)ethoxy)phenyl, benzyl, —CH₂N(CH₃)C(═O)CH₂NH₂, —CH₂N(CH₃)C(═O)CH₂CH₂NH₂, —CH₂N(CH₃)C(═O)CH₂N(CH₃)₂, —CH₂N(CH₃)C(═O)CH₂CH₂N(CH₃)₂, N-(2-(diethylamino)ethyl)carboxamido, N-(3-(diethylamino)propyl)carboxamido, N-(2-morpholinoethyl)carboxamido, N-(2-(piperazin-1-yl)ethyl)carboxamido, N-(3-(piperazin-1-yl)propyl)carboxamido, N-(3-morpholinopropyl)carboxamido, N-(2-(4-methylpiperazin-1-yl)ethyl)carboxamido, N-(3-(4-methylpiperazin-1-yl)propyl)carboxamido, (2-aminoethyl)carboxamidophenyl, (3-aminopropyl)carboxamidophenyl, (3-(diethylamino)propyl)carboxamidophenyl, (2-(piperazin-1-yl)ethyl)carboxamidophenyl, (3-(piperazin-1-yl)propy))carboxamidophenyl, (2-(diethylamino)ethyl)carboxamidophenyl, 2-(diethylamino)ethylamino, 3-(diethylamino)propylamino, 2-(dimethylamino)ethylamino, 3-(dimethylamino)propylamino, 2-aminoethylamino, 3-aminopropylamino, 2-(methylamino)ethylamino, 3-(methylamino)propylamino, 2-(2-hydroxyethylamino)ethylamino, 3-(2-hydroxyethylamino)propylamino. 2-(2-(dimethylamino)ethylamino)ethylamino, 3-(2-(dimethylamino)ethylamino)propylamino, 2-(2-(diethylamino)ethylamino)ethylamino, 3-(2-(diethylamino)ethylamino)propylamino, 2-(piperidin-1-yl)ethylamino, 3-(piperidin-1-yl)propylamino, 2-morpholinoethylamino, 3-morpholinopropylamino, 2-(piperazin-1-yl)ethylamino, 3-(piperazin-1-yl)propy)amino, 2-(4-methylpiperazin-1-yl)ethylamino, 3-(4-methylpiperazin-1-yl)propylamino, 2-(diethylamino)ethoxy, 3-(diethylamino)propoxy, 2-(dimethylamino)ethoxy, 3-(dimethylamino)propoxy, 2-aminoethoxy, 3-aminopropoxy, 2-(methylamino)ethoxy, 3-(methylamino)propoxy, 2-(2-hydroxyethylamino)ethoxy, 3-(2-hydroxyethylamino)propoxy, 2-(2-(dimethylamino)ethylamino)ethoxy, 3-(2-(dimethylamino)ethylamino)propoxy, 2-(2-(diethylamino)ethylamino)ethoxy, 3-(2-(diethylamino)ethylamino)propoxy, 2-(piperidin-1-yl)ethoxy, 3-(piperidin-1-yl)propoxy, 2-morpholinoethoxy, 3-morpholinopropoxy, 2-(piperazin-1-yl)ethoxy, 3-(piperazin-1-yl)propoxy, 2-(4-methylpiperazin-1-yl)ethoxy, 3-(4-methylpiperazin-1-yl)propoxy, (2-(diethylamino)ethylamino)methyl, (3-(diethylamino)propylamino)methyl, (2-(dimethylamino)ethylamino)methyl, (3-(dimethylamino)propy)amino)methyl, (2-aminoethylamino)methyl, (3-aminopropylamino)methyl, (2-(methylamino)ethylamino)methyl, (3-(methylamino)propylamino)methyl, (2-(2-hydroxyethylamino)ethylamino)methyl, (3-(2-hydroxyethylamino)propylamino)methyl, (2-(piperidin-1-yl)ethylamino)methyl, (3-(piperidin-1-yl)propylamino)methyl, (2-morpholinoethylamino)methyl, (3-morpholinopropylamino)methyl, 4-(2-aminoethoxy)phenyl, 4-(3-aminopropoxy)phenyl, 4-(2-(dimethylamino)ethoxy)phenyl, 4-(3-(dimethylamino)propoxy)phenyl, 4-(2-(Diethylamino)ethoxy)phenyl, 4-(3-(diethylamino)propoxy)phenyl, 4-(2-(2-hydroxyethylamino)ethoxy)phenyl, 4-(3-(2-hydroxyethylamino)propoxy)phenyl, 4-(2-(2-(dimethylamino)ethylamino)ethoxy)phenyl, 4-(3-(2-(dimethylamino)ethylamino)propoxy)phenyl, 4-(2(2-(diethylamino)ethylamino)ethoxy)phenyl, 4-(3-(2-(diethylamino)ethylamino)propoxy)phenyl, 4-(2-(piperidin-1-yl)ethoxy)phenyl, 4-(3-(piperidin-1-yl)propoxy)phenyl, 4-(2-morpholinoethoxy)phenyl, 4-(3-morpholinopropoxy)phenyl, 4-(2-(piperazin-1-yl)ethoxy)phenyl, 4-(3-(piperazin-1-yl)propoxy)phenyl, 4-(2-(4-methylpiperazin-1-yl)ethoxy)phenyl, 4-(3-(4-methylpiperazin-1-yl)propoxy)phenyl, 4-(2-(4-(2-aminoacetyl)piperazin-1-yl)ethoxy)phenyl, 4-(3-(4-(2-aminoacetyl)piperazin-1-yl)propoxy)phenyl, 4-(2-(3-aminopyrrolidin-1-yl)ethoxy)phenyl, 4-(3-(3-aminopyrrolidin-1-yl)propoxy)phenyl, 4-(2-aminoethylamino)phenyl, 4-(3-aminopropylamino)phenyl, 4-(2-(dimethylamino)ethylamino)phenyl, 4-(3-(dimethylamino)propylamino)phenyl, 4-(2-(diethylamino)ethylamino)phenyl, 4-(3-(diethylamino)propylamino)phenyl, 4-(2-(2-hydroxyethylamino)ethylamino)phenyl, 4-(3-(2-hydroxyethylamino)propylamino)phenyl, 4-(2-(piperidin-1-yl)ethylamino)phenyl, 4-(3-(piperidin-1-yl)propylamino)phenyl, 4-(2-morpholinoethylamino)phenyl, 4-(3-morpholinopropylamino)phenyl, 4-(2-(pyrrolidin-1-yl)ethylamino)phenyl, and 4-(3-(pyrrolidin-1-yl)propylamino)phenyl.

9. The method of claim 1, wherein R⁶ is selected from the group consisting of hydrogen, C₁₋₂₀ alkyl, C₁₋₂₀ alkylene-OH, optionally substituted C₁₋₂₀ alkylene-aryl, and optionally substituted C₁₋₂₀ alkylene-heteroaryl.

10. The method of claim 1, wherein R⁶ is selected from the group consisting of methyl, ethyl, 2-(piperidin-1-yl)ethyl, and (2-aminoethyl)benzyl.

11. The method of claim 1, wherein the compound having a formula II is set forth at paragraph 49.

12. A method of treating cancer comprising administering a compound having a formula I or III, or a mixture or pharmaceutically acceptable salt or hydrate thereof: 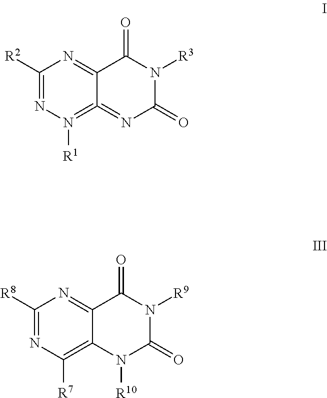
wherein R¹, R³, R⁷, R⁸, R⁹, and R¹⁰ are each independently selected from the group consisting of hydrogen, optionally substituted C₁₋₂₀ alkyl, optionally substituted C₁₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl; and
R² is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, and optionally substituted heteroaryl;
with the proviso that R² is different from hydrogen when both R¹ and R³ are methyl.

13. The method of claim 12, wherein the compound having a formula I or III is set forth at paragraph 48 or paragraph 50.

14. A compound having a formula II, or a pharmaceutically acceptable salt or hydrate thereof: 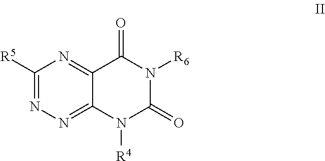
wherein R⁴ and R⁶ are independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl; and
R⁵ is selected from the group consisting of optionally substituted C₉₋₂₀ alkyl, optionally substituted C₇₋₂₀ alkenyl, optionally substituted C₂₋₂₀ alkynyl, optionally substituted C₇₋₂₀ cycloalkyl, optionally substituted C₇₋₂₀ heterocycloalkyl, optionally substituted amino(C₁₋₂₀ alkoxy)phenyl, optionally substituted amino(C₁₋₂₀ alkylamino)phenyl, optionally substituted amino(C₁₋₂₀ alkyl)carboxamidophenyl, optionally substituted amino(C₁₋₂₀ alkoxy)heteroaryl, optionally substituted amino(C₁₋₂₀ alkylamino)heteroaryl, optionally substituted amino(C₁₋₂₀ alkyl)carboxamido heteroaryl, optionally substituted amino(C₁₋₂₀ alkylamino), optionally substituted amino(C₁₋₂₀ alkoxy), optionally substituted amino(C₁₋₂₀ alkyl)carboxamido, optionally substituted amino(C₁₋₂₀ alkyl)amino(C₁₋₂₀ alkyl), and optionally substituted amino(C₁₋₂₀ alkyl)acylamino(C₁₋₂₀ alkyl).

15. The compound of claim 14, wherein R⁴ is selected from the group consisting of hydrogen, C₁₋₂₀ alkyl, C₁₋₂₀ alkylene-OH, C₁₋₂₀ alkylene-NR^{a}R^{b}, optionally substituted C₁₋₂₀ alkylene-aryl, and optionally substituted C₁₋₂₀ alkylene-heteroaryl, wherein R^{a} and R^{b} are each independently selected from the group consisting of hydrogen and C₁₋₂₀ alkyl or R^{a} and R^{b} taken together with the nitrogen atom to which they are bound form an optionally substituted 3 to 10 membered heterocyclic ring.

16. The compound of claim 14, wherein R⁴ is selected from the group consisting of methyl, ethyl, propyl, octyl, 2-(N,N-diethylamino)ethyl, phenyl, fluorophenyl, cyclopentyl, cyclopropyl, optionally substituted C₁₋₃ alkylene-aryl, and optionally substituted C₁₋₃ alkylene-heteroaryl.

17. The compound of claim 14, wherein R⁴ is selected from the group consisting of benzyl, 2-phenethyl, 3-phenpropyl, methylbenzyl, t-butylbenzyl, fluorobenzyl, difluorobenzyl, dichlorobenzyl, nitrobenzyl, trifluoromethylbenzyl, and pyridine-3-ylmethyl.

18. The compound of claim 14, wherein R⁵ is selected from the group consisting of optionally substituted amino(C₁₋₃ alkoxy)phenyl, optionally substituted amino(C₁₋₃ alkylamino)phenyl, optionally substituted amino(C₁₋₃ alkyl)carboxamidophenyl, optionally substituted amino(C₁₋₃ alkoxy)heteroaryl, optionally substituted amino(C₁₋₃ alkylamino)heteroaryl, optionally substituted amino(C₁₋₃ alkyl)carboxamido heteroaryl, optionally substituted amino(C₁₋₃ alkylamino), optionally substituted amino(C₁₋₃ alkoxy), optionally substituted amino(C₁₋₃ alkyl)carboxamido, optionally substituted amino(C₁₋₃ alkyl)amino(C₁₋₃ alkyl), and optionally substituted amino(C₁₋₃ alkyl)acylamino(C₁₋₃ alkyl).

19. The compound of claim 14, wherein R⁵ is selected from the group consisting of optionally substituted R^{e}R^{f}N—(C₁₋₃ alkoxy)phenyl, optionally substituted R^{e}R^{f}N—(C₁₋₃ alkylamino)phenyl, optionally substituted R^{e}R^{f}N—(C₁₋₃ alkyl)carboxamidophenyl, optionally substituted R^{e}R^{f}N—(C₁₋₃ alkoxy)heteroaryl, optionally substituted R^{e}R^{f}N—(C₁₋₃ alkylamino)heteroaryl, optionally substituted R^{e}R^{f}N—(C₁₋₃ alkyl)carboxamido heteroaryl, optionally substituted R^{e}R^{f}N—(C₁₋₃ alkylamino), optionally substituted R^{e}R^{f}N—(C₁₋₃ alkoxy), optionally substituted R^{e}R^{f}N—(C₁₋₃ alkyl)carboxamido, optionally substituted R^{e}R^{f}N—(C₁₋₃ alkyl)amino(C₁₋₃ alkyl), and optionally substituted R^{e}R^{f}N—(C₁₋₃ alkyl)acylamino(C₁₋₃ alkyl);
R^{e} and R^{f} are independently selected from the group consisting of hydrogen, optionally substituted C₁₋₃ alkyl, and optionally substituted C₁₋₃ aminoalkyl; or
R^{e} and R^{f} taken together with the nitrogen atom to which they are bound form an optionally substituted 3 to 6 membered heterocyclic ring.

20. The compound of claim 14, wherein is selected from the group consisting of trifluoromethylphenyl, carboxyphenyl, (2-(4-aminoacetylpiperazin-1-yl)ethoxy)phenyl, (2-(4-(dimethylamino)piperidin-1-yl)ethoxy)phenyl, (2-(3-aminopyrrolidin-1-yl)ethoxy)phenyl, (3-(morpholin-4-yl)propyloxy)phenyl, (2-(piperidin-1-yl)ethoxy)phenyl, (2-(dimethylamino)ethyl)phenyl, (2-(piperidin-1-yl)ethylamino)phenyl, (2-(diethylamino)ethylcarboxamide)phenyl, (2-(4-methylpiperazin-1-yl)ethoxy)phenyl, (3-(4-methylpiperazin-1-yl)propyl)phenyl, 2-(N,N-diethylamino)ethoxy)phenyl, (2-(morpholin-4-yl)ethoxy)phenyl, benzyl, —CH₂N(CH₃)C(═O)CH₂NH₂, —CH₂N(CH₃)C(═O)CH₂CH₂NH₂, —CH₂N(CH₃)C(═O)CH₂N(CH₃)₂, —CH₂N(CH₃)C(═O)CH₂CH₂N(CH₃)₂, N-(2-(diethylamino)ethyl)carboxamido, N-(3-(diethylamino)propyl)carboxamido, N-(2-morpholinoethyl)carboxamido, N-(2-(piperazin-1-yl)ethyl)carboxamido, N-(3-(piperazin-1-yl)propyl)carboxamido, N-(3-morpholinopropyl)carboxamido, N-(2-(4-methylpiperazin-1-yl)ethyl)carboxamido, N-(3-(4-methylpiperazin-1-yl)propyl)carboxamido, (2-aminoethyl)carboxamidophenyl, (3-aminopropyl)carboxamidophenyl, (3-(diethylamino)propyl)carboxamidophenyl, (2-(piperazin-1-yl)ethyl)carboxamidophenyl, (3-(piperazin-1-yl)propyl)carboxamidophenyl, (2-(diethylamino)ethyl)carboxamidophenyl, 2-(diethylamino)ethylamino, 3-(diethylamino)propylamino, 2-(dimethylamino)ethylamino, 3-(dimethylamino)propylamino, 2-aminoethylamino, 3-aminopropylamino, 2-(methylamino)ethylamino, 3-(methylamino)propylamino, 2-(2-hydroxyethylamino)ethylamino, 3-(2-hydroxyethylamino)propylamino, 2-(2-(dimethylamino)ethylamino)ethylamino, 3-(2-(dimethylamino)ethylamino)propylamino, 2-(2-(diethylamino)ethylamino)ethylamino, 3-(2-(diethylamino)ethylamino)propylamino, 2-(piperidin-1-yl)ethylamino, 3-(piperidin-1-yl)propylamino, 2-morpholinoethylamino, 3-morpholinopropylamino, 2-(piperazin-1-yl)ethylamino, 3-(piperazin-1-yl)propylamino, 2-(4-methylpiperazin-1-yl)ethylamino, 3-(4-methylpiperazin-1-yl)propylamino, 2-(diethylamino)ethoxy, 3-(diethylamino)propoxy, 2-(dimethylamino)ethoxy, 3-(dimethylamino)propoxy, 2-aminoethoxy, 3-aminopropoxy, 2-(methylamino)ethoxy, 3-(methylamino)propoxy, 2-(2-hydroxyethylamino)ethoxy, 3-(2-hydroxyethylamino)propoxy, 2-(2-(dimethylamino)ethylamino)ethoxy, 3-(2-(dimethylamino)ethylamino)propoxy, 2-(2-(diethylamino)ethylamino)ethoxy, 3-(2-(diethylamino)ethylamino)propoxy, 2-(piperidin-1-yl)ethoxy, 3-(piperidin-1-yl)propoxy, 2-morpholinoethoxy, 3-morpholinopropoxy, 2-(piperazin-1-yl)ethoxy, 3-(piperazin-1-yl)propoxy, 2-(4-methylpiperazin-1-yl)ethoxy, 3-(4-methylpiperazin-1-yl)propoxy, (2-(diethylamino)ethylamino)methyl, (3-(diethylamino)propylamino)methyl, (2-(dimethylamino)ethylamino)methyl, (3-(dimethylamino)propylamino)methyl, (2-aminoethylamino)methyl, (3-aminopropylamino)methyl, (2-(methylamino)ethylamino)methyl, (3-(methylamino)propylamino)methyl, (2-(2-hydroxyethylamino)ethylamino)methyl, (3-(2-hydroxyethylamino)propylamino)methyl, (2-(piperidin-1-yl)ethylamino)methyl, (3-(piperidin-1-yl)propylamino)methyl, (2-morpholinoethylamino)methyl, (3-morpholinopropylamino)methyl, 4-(2-aminoethoxy)phenyl, 4-(3-aminopropoxy)phenyl, 4-(2-(dimethylamino)ethoxy)phenyl, 4-(3-(dimethylamino)propoxy)phenyl, 4-(2-(Diethylamino)ethoxy)phenyl, 4-(3-(diethylamino)propoxy)phenyl), 4-(2-(2-hydroxyethylamino)ethoxy)phenyl, 4-(3-(2-hydroxyethylamino)propoxy)phenyl, 4-(2-(2-(dimethylamino)ethylamino)ethoxy)phenyl, 4-(3-(2-(dimethylamino)ethylamino)propoxy)phenyl, 4-(2-(2-(diethylamino)ethylamino)ethoxy)phenyl, 4-(3-(2-(diethylamino)ethylamino)propoxy)phenyl, 4-(2-(piperidin-1-yl)ethoxy)phenyl, 4-(3-(piperidin-1-yl)propoxy)phenyl, 4-(2-morpholinoethoxy)phenyl, 4-(3-morpholinopropoxy)phenyl, 4-(2-(piperazin-1-yl)ethoxy)phenyl, 4-(3-(piperazin-1-yl)propoxy)phenyl, 4-(2-(4-methylpiperazin-1-yl)ethoxy)phenyl, 4-(3-(4-methylpiperazin-1-yl)propoxy)phenyl, 4-(2-(4-(2-aminoacetyl)piperazin-1-yl)ethoxy)phenyl, 4-(3-(4-(2-aminoacetyl)piperazin-1-yl)propoxy)phenyl, 4-(2-(3-aminopyrrolidin-1-yl)ethoxy)phenyl, 4-(3-(3-aminopyrrolidin-1-yl)propoxy)phenyl, 4(2-aminoethylamino)phenyl, 4-(3-aminopropylamino)phenyl, 4-(2-(dimethylamino)ethylamino)phenyl, 4-(3-(dimethylamino)propylamino)phenyl, 4-(2-(diethylamino)ethylamino)phenyl, 4-(3-(diethylamino)propylamino)phenyl, 4-(2-(2-hydroxyethylamino)ethylamino)phenyl, 4-(3-(2-hydroxyethylamino)propylamino)phenyl, 4-(2-(piperidin-1-yl)ethylamino)phenyl, 4-(3-(piperidin-1-yl)propylamino)phenyl, 4-(2-morpholinoethylamino)phenyl, 4-(3-morpholinopropylamino)phenyl, 4-(2-(pyrrolidin-1-yl)ethylamino)phenyl, and 4-(3-(pyrrolidin-1-yl)propylamino)phenyl.

21. The compound of claim 14, wherein R⁶ is selected from the group consisting of hydrogen, C₁₋₂₀ alkyl, C₁₋₂₀ alkylene-OH, optionally substituted C₁₋₂₀ alkylene-aryl, and optionally substituted C₁₋₂₀ alkylene-heteroaryl.

22. The compound of claim 14, wherein R⁶ is selected from the group consisting of methyl, ethyl, 2-(piperidin-1-yl)ethyl, and (2-aminoethyl)benzyl.

23. A compound having a formula II, or a pharmaceutically acceptable salt or hydrate thereof: 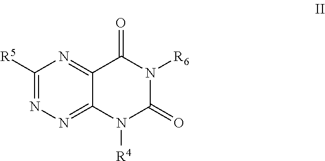
wherein R⁴ is selected from the group consisting of C₇₋₂₀ alkyl, substituted C₁₋₂₀ alkyl, C₉₋₂₀ alkenyl, substituted C₂₋₂₀ alkenyl, optionally substituted C₂₋₂₀ alkynyl, C₇₋₂₀ cycloalkyl, substituted C₃₋₂₀ cycloalkyl, optionally substituted C₂₋₂₀ heterocycloalkyl, substituted aryl, and optionally substituted heteroaryl;
with the proviso that R⁴ is different from benzyl;
R⁵ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, optionally substituted heteroaryl, optionally substituted C₁₋₂₀ alkylamino, optionally substituted C₁₋₂₀ alkoxy, and optionally substituted C₁₋₂₀ alkylcarboxamido; and
R⁶ is selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl.

24. A compound having a formula I or III, or a pharmaceutically acceptable salt or hydrate thereof: 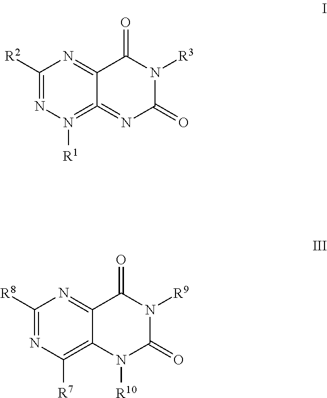
wherein R¹, R², and R³ are each independently selected from the group consisting of optionally substituted aryl and optionally substituted heteroaryl;
R⁷, R⁸, R⁹, and R¹⁰ are each independently selected from the group consisting of hydrogen, optionally substituted C₁₋₂₀ alkyl, optionally substituted C₂₋₂₀ alkenyl, optionally substituted C₂₋₂₀ alkynyl, optionally substituted C₃₋₂₀ cycloalkyl, optionally substituted C₂₋₂₀ heterocycloalkyl, optionally substituted aryl, and optionally substituted heteroaryl;
with the proviso that when R⁷ is hydrogen and R⁸ is phenyl, para-methoxyphenyl, furyl, or —CO₂Et, at least one of R⁹ and R¹⁰ is different from methyl.

25. A compound as set forth at paragraph 48, paragraph 49, or paragraph 50.

26. The compound of claim 25 selected from the group consisting of:
3-(4-(2-(Diethylamino)ethoxy)phenyl)-8-(3,4-difluorobenzyl)-6-methylpyrimido[5,4-e][1,2,4]triazine-5,7(6H,8H)-dione;
8-(3,4-Difluorobenzyl)-6-methyl-3-(4-(2-(piperazin-1-yl)ethoxy)phenyl)pyrimido[5,4-e][1,2,4]triazine-5,7(6H, 8H)-dione;
3-(4-(2-(Diethylamino)ethylamino)phenyl)-8-(3,4-difluorobenzyl)-6-methylpyrimido[5,4-e][1,2,4]triazine-5,7(6H,8H)-dione;
8-(3,4-Difluorobenzyl)-6-methyl-3-(4-(2-(piperidin-1-yl)ethylamino)phenyl)pyrimido[5,4-e][1,2,4]triazine-5,7(6H,8H)-dione;
8-(3,4-Difluorobenzyl)-6-methyl-3-(3-(piperazin-1-yl)propylamino)pyrimido[5,4-e][1,2,4]triazine-5,7(6H,8H)-dione;
8-(3,4-Difluorobenzyl)-6-methyl-3-(3-(piperazin-1-yl)propoxy)pyrimido[5,4-e][1,2,4]triazine-5,7(6H,8H)-dione;
3-((3-Aminopropylamino)methyl)-8-(3,4-difluorobenzyl)-6-methylpyrimido[5,4-e][1,2,4]triazine-5,7(6H,8H)-dione; and
1-(4-Fluorobenzyl)-3-methyl-6-(4-(2-(4-methylpiperazin-1-yl)ethoxy)phenyl)pyrimido[5,4-d]pyrimidine-2,4(1H,3H)-dione.
